SEQUENCE LISTING (31 sequences)

SEQ ID NO: 1 - protein, 393 aa, Cupriavidus necator
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 2 - protein, 388 aa, Staphylococcus aureus
MTIGIDKINFYVPKYYVDMAKLAEARQVDPNKFLIGIGQTEMAVSPVNQDIVSMGANAAKDIITDEDKKKIGMVIVATESAVDAAKAAAVQIHNLLGIQPFARCFEMKEACYAATPAIQLAKDYLATRPNEKVLVIATDTARYGLNSGGEPTQGAGAVAMVIAHNPSILALNEDAVAYTEDVYDFWRPTGHKYPLVDGALSKDAYIRSFQQSWNEYAKRQGKSLADFASLCFHVPFTKMGKKALESIIDNADETTQERLRSGYEDAVDYNRYVGNIYTGSLYLSLISLLENRDLQAGETIGLFSYGSGSVGEFYSATLVEGYKDHLDQAAHKALLNNRTEVSVDAYETFFKRFDDVEFDEEQDAVHEDRHIFYLSNIENNVREYHRPE

SEQ ID NO: 3 - protein, 425 aa, Staphylococcus aureus
MQSLDKNFRHLSRQQKLQQLVDKQWLSEDQFDILLNHPLIDEEVANSLIENVIAQGALPVGLLPNIIVDDKAYVVPMMVEEPSVVAAASYGAKLVNQTGGFKTVSSERIMIGQIVFDGVDDTEKLSADIKALEKQIHKIADEAYPSIKARGGGYQRIAIDTFPEQQLLSLKVFVDTKDAMGANMLNTILEAITAFLKNESPQSDILMSILSNHATASVVKVQGEIDVKDLARGERTGEEVAKRMERASVLAQVDIHRAATHNKGVMNGIHAVVLATGNDTRGAEASAHAYASRDGQYRGIATWRYDQKRQRLIGTIEVPMTLAIVGGGTKVLPIAKASLELLNVDSAQELGHVVAAVGLAQNFAACRALVSEGIQQGHMSLQYKSLAIVVGAKGDEIAQVAEALKQEPRANTQVAERILQEIRQQ

SEQ ID NO: 4 - protein, 279 aa, Staphylococcus aureus
MAVPFNAGKIKVLIEALESGNYSSIKSDVYDGMLYDAPDHLKSLVNRFVELNNITEPLAVTIQTNLPPSRGLGSSAAVAVAFVRASYDFLGKSLTKEELIEKANWAEQIAHGKPSGIDTQTIVSGKPVWFQKGHAETLKTLSLDGYMVVIDTGVKGSTRQAVEDVHKLCEDPQYMSHVKHIGKLVLRASDVIEHHNFEALADIFNECHADLKALTVSHDKIEQLMKIGKENGAIAGKLTGAGRGGSMLLLAKDLPTAKNIVKAVEKAGAAHTWIENLGG

SEQ ID NO: 5 - protein, 358 aa, Staphylococcus aureus
MIQVKAPGKLYIAGEYAVTEPGYKSVLIALDRFVTATIEEADQYKGTIHSKALHHNPVTFSRDEDSIVISDPHAAKQLNYVVTAIEIFEQYAKSCDIAMKHFHLTIDSNLDDSNGHKYGLGSSAAVLVSVIKVLNEFYDMKLSNLYIYKLAVIANMKLQSLSSCGDIAVSVYSGWLAYSTFDHEWVKHQIEDTTVEEVLIKNWPGLHIEPLQAPENMEVLIGWTGSPASSPHFVSEVKRLKSDPSFYGDFLEDSHRCVEKLIHAFKTNNIKGVQKMVRQNRTIIQRMDKEATVDIETEKLKYLCDIAEKYHGASKTSGAGGGDCGITIINKDVDKEKIYDEWTKHGIKPLKFNIYHGQ

SEQ ID NO: 6 - protein, 344 aa, Streptococcus pneumoniae
MYHSLGNQFDTRTRTSRKIRRERSCSDMDREPVTVRSYANIAIIKYWGKKKEKEMVPATSSISLTLENMYTETTLSPLPANVTADEFYINGQLQNEVEHAKMSKIIDRYRPAGEGFVRIDTQNNMPTAAGLSSSSSGLSALVKACNAYFKLGLDRSQLAQEAKFASGSSSRSFYGPLGAWDKDSGEIYPVETDLKLAMIMLVLEDKKKPISSRDGMKLCVETSTTFDDWVRQSEKDYQDMLIYLKENDFAKIGELTEKNALAMHATTKTASPAFSYLTDASYEAMDFVRQLREKGEACYFTMDAGPNVKVFCQEKDLEHLSEIFGQRYRLIVSKTKDLSQDDCC

SEQ ID NO: 7 - protein, 176 aa, Burkholderia multivorans
MEERLILVDTDDRPIGICEKMRAHHEGLLHRAFSIFVFDSAGRLLLQQRALNKYHSGGLWSNTCCGHPRPREALPDAVRRRLGEEMGFACELRPVDALVYRARFENDLIEHEFVHIHVGRFDGTVAPDFAEVAAWRWIDVPTLLEWMADEPSAFTVWFHCMIERAGLPVLHRWAHR

SEQ ID NO: 8 - protein, 594 aa, Mucuna pruriens
MATNPSCLSTPFLSSTPALSTRFPLSENFTQKTSLVNPKPWPLISAVSSQFSQIAEDNSRRSANYHPNLWDFEFLQSLENDSKMEKLEEKATKLEEEVRNMMNEAKTEALSLLELIDDVQRLGLTYKFEKDIIKALEKIVPLDESGLHVTSLSFRILRQHGFEVSQDVFKRFKDKEGGFCAELKDDVQGLLSLYEASYLGFEGESLLDEARAFSITHLKNNLNKGINTKVAQQVSHALELPYHRRLHRLEARWLLDKYEPKEPHHHLLHELAKLDFNLVQSLYQKELRELSLWWREIGLTSKLDFVRDRLMEVYFWALGMAPDPQFSECRKVVTKMFGLVTIIDDVYDVYGTLDELQLFTDAVERWDVNAINTLPDYMKLCYLALYNTVNDTAYSILKEKGHNNISYLTKSWCELCKAFLQEAKWSNNKIIPAFNKYLDNASVSSSGVALLAPSYFLVCQEQDISDQALHSLTNFHGLVRSSCTIFRLCNDLATSSAELERGETTNSITSYMHENETSEEQACKELRNLIDAEWKKMNEERVSNSTLPKAFREIAINMARISHCTYQYGDGLGRPDYTTENRIKLLLIDPFPIN

SEQ ID NO: 9 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADPESLIDKLQVREAEVFQQAELSYPSIVKRGGGLRDLQYRTFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRAMAILNDLRKQ

SEQ ID NO: 10 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIVYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 11 - protein, 292 aa, Streptococcus pneumoniae
MTKKVGVGQAHSKIILIGEHAVVYGYPAISLPLLEVEVTCKVVPAESPWRLYEEDTLSMAVYASLEYLNITEACIRCEIDSAIPEKRGMGSSAAISIAAIRAVFDYYQADLPHDVLEILVNRAEMIAHMNPSGLDAKTCLSDQPIRFIKNVGFTELEMDLSAYLVIADTGVYGHTREAIQVVQNKGKDALPFLHALGELTQQAEVAISQKDAEGLGQILSQAHLHLKEIGVSSPEADFLVETTLSHGALGAKMSGGGLGGCIIALVTNLTHAQELAERLEEKGAVQTWIESL

SEQ ID NO: 12 - protein, 335 aa, Streptococcus pneumoniae
MIAVKTCGKLYWAGEYAILEPGQLALIKDIPIYMRAEIAFSDSYRIYSDMFDFAVDLRPNPDYSLIQETIALMGDFLAVRGQNLRPFSLEICGKMEREGKKFGLGSSGSVVVLVVKALLALYDVSVDQELLFKLTSAVLLKRGDNGSMGDLACIVAEDLVLYQSFDRQKVAAWLEEENLATVLERDWGFSISQVKPTLECDFLVGWTKEVAVSSHMVQQIKQNINQNFLTSSKETVTSLVEALEQGKSEKIIDQVEVASKLLEGLSTDIYTPLLRQLKEASQDLQTVAKSSGAGGGDCGIALSFDAQSTKTLKNRWADLGIELLYQERIGHDDKS

SEQ ID NO: 13 - protein, 317 aa, Streptococcus pneumoniae
MDREPVTVRSYANIAIIKYWGKKKEKEMVPATSSISLTLENMYTETTLSPLPANVTADEFYINGQLQNEVEHAKMSKIIDRYRPAGEGFVRIDTQNNMPTAAGLSSSSSGLSALVKACNAYFKLGLDRSQLAQEAKFASGSSSRSFYGPLGAWDKDSGEIYPVETDLKLAMIMLVLEDKKKPISSRDGMKLCVETSTTFDDWVRQSEKDYQDMLIYLKENDFAKIGELTEKNALAMHATTKTASPAFSYLTDASYEAMAFVRQLREKGEACYFTMDAGPNVKVFCQEKDLEHLSEIFGHRYRLIVSKTKDLSQDDCC

SEQ ID NO: 14 - protein, 336 aa, Streptococcus pneumoniae
MTTNRKDEHILYALEQKSSYNSFDEVELIHSSLPLYNLDEIDLSTEFAGRKWDFPFYINAMTGGSNKGREINQKLAQVAESCGILFVTGSYSAALKNPTDDSFSVKSSHPNLLLGTNIGLDKPVELGLQTVEEMNPVLLQVHVNVMQELLMPEGERKFRSWQSHLADYSKQIPVPIVLKEVGFGMDAKTIERAYEFGVRTVDLSGRGGTSFAYIENRRSGQRDYLNQWGQSTMQALLNAQEWKDKVELLVSGGVRNPLDMIKCLVFGAKAVGLSRTVLELVETYTVEEVIGIVQGWKADLRLIMCSLNCATIADLQKVDYLLYGKLKEAKDQMKKA

SEQ ID NO: 15 - protein, 559 aa, Populus alba
RCSVSTENVSFTETETEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 16 - DNA, 1182 bp, Cupriavidus necator
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataa

SEQ ID NO: 17 - DNA, 1167 bp, Staphylococcus aureus
atgacaataggtatcgacaaaataaacttttacgttccaaagtactatgtagacatggctaaattagcagaagcacgccaagtagacccaaacaaatttttaattggaattggtcaaactgaaatggctgttagtcctgtaaaccaagacatcgtttcaatgggcgctaacgctgctaaggacattataacagacgaagataaaaagaaaattggtatggtaattgtggcaactgaatcagcagttgatgctgctaaagcagccgctgttcaaattcacaacttattaggtattcaaccttttgcacgttgctttgaaatgaaagaagcttgttatgctgcaacaccagcaattcaattagctaaagattatttagcaactagaccgaatgaaaaagtattagttattgctacagatacagcacgttatggattgaattcaggcggcgagccaacacaaggtgctggcgcagttgcgatggttattgcacataatccaagcattttggcattaaatgaagatgctgttgcttacactgaagacgtttatgatttctggcgtccaactggacataaatatccattagttgatggtgcattatctaaagatgcttatatccgctcattccaacaaagctggaatgaatacgcaaaacgtcaaggtaagtcgctagctgacttcgcatctctatgcttccatgttccatttacaaaaatgggtaaaaaggcattagagtcaatcattgataacgctgatgaaacaactcaagagcgtttacgttcaggatatgaagatgctgtagattataaccgttatgtcggtaatatttatactggatcattatatttaagcctaatatcattacttgaaaatcgtgatttacaagctggtgaaacaatcggtttattcagttatggctcaggttcagttggtgaattttatagtgcgacattagttgaaggctacaaagatcatttagatcaagctgcacataaagcattattaaataaccgtactgaagtatctgttgatgcatatgaaacattcttcaaacgttttgatgacgttgaatttgacgaagaacaagatgctgttcatgaagatcgtcatattttctacttatcaaatattgaaaataacgttcgcgaatatcacagaccagagtaa

SEQ ID NO: 18 - DNA, 1278 bp, Staphylococcus aureus
ctattgttgtctaatttcttgtaaaatgcgttcagctacttgtgtattcgcacggggttcttgcttcaatgcttcagctacttgcgcaatttcatcaccttttgcacctacaacaatagctaaagatttatattgcaagctcatatggccttgctggataccttcggaaacgagcgcgcgacatgctgcaaagttctgtgctaaaccaacggcagcaactacatgacctaattcttgtgctgaatctacatttagcaattctaaagaagctttagcaattggtaatacttttgtaccaccgccaacgattgccaatgtcataggcacttctattgtaccaattaaacgttgacgtttttgatcgtatctccatgttgcaataccacgatactgtccgtcacgactcgcgtatgcatgcgcacttgcttctgcaccacgcgtatcatttcctgttgctaaaacaacggcatgtatgccattcataacacctttattatgtgttgcagcacgatgaatatcaacttgtgccaatacagaagcacgttccattcgtttggcaacctcttctccagttctctcgccccttgctaaatctttaacgtcaatttcgccttgaactttaacaacggacgctgttgcatgattggataaaatactcattaaaatgtcgctttgtggagattcattttttaaaaatgcagttatggcctctaaaatcgtattaagcatattagcgcccatagcatctttcgtatcaacaaatacttttaaagatagtaactgttgctcaggaaatgtatcaatagctatacgttggtaaccaccaccacgcgctttaatagaaggatatgcctcatccgcaattttatgaatttgcttttctaaagctttaatgtctgctgataatttttcagtatcgtcaacgccatcaaagacgatttgacctatcataatacgttcagaagataccgttttaaatccgccagtctgattcactagctttgcaccataactagctgcagcgacaactgaaggctcttccaccatcataggtacaacatatgccttatcgtccacaatgatattcggtaataatccaacgggtaatgcaccttgcgcgatgacattttcaattaaactatttgctacttcctcatcaattaatggatgattcaataaaatgtcgaattgatcttctgataaccattgcttatctaccaattgttgtaacttttgttgacgagataaatgtcggaaattcttatctaaactttgcat

SEQ ID NO: 19 - DNA, 840 bp, Staphylococcus aureus
attgcagtaccgtttaacgcaggtaaaatcaaagttttaatagaagccttagagagcgggaactattcgtctattaaaagcgatgtttacgatggtatgttatatgatgcgcctgaccatcttaagtctttggtgaaccgttttgtagaattaaataatattacagagccgctagcagtaacgatccaaacgaatttaccaccatcacgtggattaggatcgagtgcagctgtcgcggttgcttttgttcgtgcaagttatgattttttagggaaatcattaacgaaagaagaactcattgaaaaggctaattgggcagagcaaattgcacatggtaaaccaagtggtattgatacgcaaacgattgtatcaggcaaaccagtttggttccaaaaaggtcatgctgaaacattgaaaacgttaagtttagacggctatatggttgttattgatactggtgtgaaaggttcaacaagacaagcggtagaagatgttcataaactttgtgaggatcctcagtacatgtcacatgtaaaacatatcggtaagttagttttacgtgcgagtgatgtgattgaacatcataactttgaagccctagcggatatttttaatgaatgtcatgcggatttaaaggcgttgacagttagtcatgataaaatagaacaattaatgaaaattggtaaagaaaatggtgcgattgctggaaaacttactggtgctggtcgtggtggaagtatgttattgcttgccaaagatttaccaacagcgaaaaatattgtgaaagctgtagaaaaagctggtgcagcacatacatggattgagaatttaggaggttaa

SEQ ID NO: 20 - DNA, 1077 bp, Staphylococcus aureus
atgattcaggtcaaagcacccggaaaactttatattgctggagaatatgctgtaacagaaccaggatataaatctgtacttattgcgttagatcgttttgtaactgctactattgaagaagcagaccaatataaaggtaccattcattcaaaagcattacatcataacccagttacatttagtagagatgaagatagtattgtcatttcagatccacatgcagcaaaacaattaaattatgtggtcacagctattgaaatatttgaacaatacgcgaaaagttgcgatatagcgatgaagcattttcatctgactattgatagtaatttagatgattcaaatggtcataaatatggattaggttcaagtgcagcagtacttgtgtcagttataaaagtattaaatgaattttatgatatgaagttatctaatttatacatttataaactagcagtgattgcaaatatgaagttacaaagtttaagttcatgcggagatattgctgtgagtgtatatagtggatggttagcgtatagtacttttgatcatgaatgggttaagcatcaaattgaagatactacggttgaagaagttttaatcaaaaactggcctggattgcacatcgaaccattacaagcacctgaaaatatggaagtacttatcggttggactggctcaccggcgtcatcaccacactttgttagcgaagtgaaacgtttgaaatcagatccttcattttacggtgacttcttagaagattcacatcgttgtgttgaaaagcttattcatgcttttaaaacaaataacattaaaggtgtgcaaaagatggtgcgtcagaatcgtacaattattcaacgtatggataaagaagctacagttgatatagaaactgaaaagctaaaatatttgtgtgatattgctgaaaagtatcacggtgcatctaaaacatcaggcgctggtggtggagactgtggtattacaattatcaataaagatgtagataaagaaaaaatttatgatgaatggacaaaacatggtattaaaccattaaaatttaatatttatcatgggcaataa

SEQ ID NO: 21 - DNA, 1035 bp, Streptococcus pneumoniae
ttgtatcatagccttggtaaccaatttgacacacgcacaagaactagcagaaagattagaagagaaaggagctgttcagacatggatagagagcctgtgacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttagcgcaggaagccaagtttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgactttcgacgactgggttcgtcagtctgagaaggactatcaggatatgctgatttatctcaaggaaaatgattttgccaagattggagaattaacggagaaaaatgccctggctatgcatgctacgacaaagactgctagtccagccttttcttatctgacggatgcctcttatgaggctatggactttgttcgtcagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaa

SEQ ID NO: 22 - DNA, 531 bp, Burkholderia multivorans
tcatctgtgtgcccagcgatgcagcactggcaatccggctcgctctatcatgcagtggaaccagacagtaaaagcgctcggctcgtccgccatccactccagcaaggttggcacatcgatccatcgccacgccgctacttccgcgaaatctggggcgaccgttccatcgaaccgaccaacatgaatatgcacaaactcgtgctcgatcaggtcgttctcaaatctcgcgcggtacacgagcgcgtccacgggccgaagttcacatgcgaatcccatttcttcgccaagccggcggcgaaccgcatcaggcagcgcttcgcgtggacgcgggtgcccgcagcatgtgttggaccacagcccgcccgagtggtacttattcagcgcacgctgctgtagcagcaagcgaccggccgagtcgaacacaaaaatcgagaatgcgcggtgcagcagcccttcatggtgcgcgcgcatcttctcgcatattcctatcggtcgatcgtcggtatcgacgaggatcaggcgttcttccat

SEQ ID NO: 23 - DNA, 1785 bp, Mucuna pruriens
atggcaaccaacccttcatgcttatctactccatttttgtcctccacaccagcactaagtactagatttccattaagtgagaacttcacacaaaaaacatctcttgtcaatcccaaaccttggccacttatttctgcagtcagctctcaatttagccaaatagcagaagataatagtcgtcgttcagctaattaccacccaaacctctgggattttgaatttctgcagtctctcgaaaatgactctaagatggaaaagctggaagagaaagcaacaaagttggaggaggaagtgcgaaacatgatgaacgaagcaaagacagaagcactaagcttattggaattgatagacgacgtccagcgtctgggattgacctacaagtttgagaaggacataatcaaagcccttgagaagattgttccattggatgagagtgggctgcatgttacttctctcagcttccgtatacttagacaacatggctttgaggtttcccaagatgtgtttaagagatttaaggacaaggagggaggtttttgtgctgaacttaaagacgatgttcaagggttgctaagtctatatgaagcatcctatcttggttttgagggagaaagtctcttagacgaggcaagggcattttcaataacacatctcaagaacaacctaaacaaaggaataaacaccaaagtagcccaacaagttagccatgcactggaacttccttatcatcgaagactgcatagactggaagcacgatggctccttgacaaatatgaaccaaaggaaccccaccatcatttactacacgagcttgcaaagttggatttcaatttggtccaatcattgtaccagaaagagttgcgagaattgtcactgtggtggagggagattgggctcacaagcaagttggactttgttcgagacagattaatggaagtgtacttttgggcgctgggaatggcacctgatcctcaatttagtgaatgtcgtaaagtcgtcactaaaatgtttgggctagttactatcatcgatgatgtatatgacgtttacggtactttggacgagctacaactcttcaccgatgctgttgagagatgggacgtgaatgcgataaatacacttccagactatatgaaattgtgctatttagccctttataacaccgtcaatgacacagcttatagcatccttaaagaaaagggacataacaacatttcttatttgacaaaatcttggtgtgagttgtgcaaagcattcctccaagaagcaaaatggtcaaacaacaaaatcattccagcattcaacaagtacctagacaatgcatcggtgtcctcctctggtgtggctttgcttgctccttcctacttcttagtgtgccaagaacaagacatttcagaccaagctcttcattccttaactaatttccatggccttgtgcgttcatcatgcaccatttttaggctttgcaatgatctggctacctcatcggctgagctagagagaggtgaaacaacaaattcaatcacatcgtacatgcatgagaatgagacttctgaggagcaagcatgtaaggagttgagaaatttgatcgatgcagagtggaagaagatgaatgaagagcgagtttcaaattctacactcccaaaagcatttagggaaatagctattaacatggctcggatttcccattgcacataccaatatggagacggacttggaaggcccgactacaccacagagaacaggataaagttgctactaatagacccttttccaattaattag

SEQ ID NO: 24 - DNA, 2412 bp, Enterococcus faecalis
atgaaaaccgtggtcatcatcgatgccctgcgcaccccgatcggcaagtataagggctccctctcccaagtgtcggccgtggacctgggtacccacgtgaccacccaactcctgaagcgccatagcacgatctccgaagagatcgaccaggtgatctttggcaacgtgctccaggccggcaacggccagaacccggcccgccagatcgccatcaactccggcctgagccacgaaatccccgccatgaccgtgaacgaagtctgcggctcgggcatgaaggccgtcatcctggcgaagcagctcatccagctcggcgaagcggaagtgctgatcgccggcggcatcgagaatatgtcgcaggcgccgaagctgcagcgcttcaactatgaaaccgagtcgtacgacgcgccgttcagctccatgatgtacgacggcctgacggacgccttctccggccaagccatgggcctgacggcggaaaacgtggccgagaagtaccacgtgacgcgcgaggaacaggaccagttctcggtccattcgcagctgaaggccgcccaggcccaggccgagggcatctttgcggacgagatcgcgccgctggaggtcagcggcaccctggtggaaaaggacgaaggcattcgccccaactcctcggtcgagaagctgggcaccctcaagaccgtgttcaaggaggacggcaccgtcaccgcgggcaatgcctcgaccatcaacgacggcgcgtcggccctcatcatcgcgagccaggaatacgcggaagcgcatggcctgccgtacctcgcgatcatccgtgactccgtggaagtcggcatcgacccggcgtacatgggcatctcccccatcaaggccatccaaaagctcctggcgcgcaaccagctgacgacggaggagatcgacctgtacgagatcaacgaagcgttcgcggcgacgagcatcgtggtgcagcgcgagctggccctgccggaggaaaaggtgaatatctacggcggcggcatttcgctgggccatgcgatcggcgcgaccggcgcccgcctgctgaccagcctgtcgtatcaactcaatcaaaaggaaaagaagtacggcgtggcgtcgctgtgcatcggcggtggcctgggcctcgccatgctgctggagcgcccgcagcagaagaagaactcgcgcttttaccagatgtcgcccgaggaacggctggcgtcgctcctgaacgaaggccaaatctcggccgataccaagaaggagttcgaaaacaccgccctgtcgagccagatcgcgaaccacatgatcgaaaatcagatcagcgaaaccgaagtgccgatgggcgtgggcctccatctgaccgtggacgaaacggactatctggtcccgatggccaccgaggaaccgtcggtgatcgccgcgctgtccaacggcgccaagatcgcccagggcttcaagacggtgaaccagcagcgcctgatgcgcggtcagatcgtgttctacgatgtggcggacccggagtcgctgatcgacaagctccaggtgcgtgaagccgaagtgttccagcaagccgaactgtcgtaccccagcatcgtcaagcgcggcggcggcctccgcgatctccagtaccgcaccttcgacgagtcgttcgtgtcggtcgattttctggtggatgtgaaggacgccatgggtgcgaacatcgtcaacgccatgctggaaggcgtcgccgaactgttccgggagtggttcgccgagcagaagatcctgttcagcatcctctcgaactacgccaccgagtccgtggtgaccatgaaaaccgccattcccgtcagccgcctgtcgaagggcagcaacggccgcgagatcgcggaaaagatcgtcctcgcctcccgctacgcgtcgctggacccgtatcgcgcggtcacccacaacaagggcattatgaacggcatcgaggccgtcgtgctggccaccggcaatgacacgcgcgccgtgtcggccagctgccatgccttcgccgtgaaggaaggccggtaccaaggcctgaccagctggacgctggacggcgaacagctgatcggcgaaatcagcgtgcccctggccctggcgaccgtgggcggcgcgaccaaggtcctgcccaagagccaggccgcggccgatctgctggccgtgaccgatgccaaggagctgtcccgcgtggtcgccgcggtgggtctggcgcagaatctggccgccctgcgggcgctggtcagcgagggcatccaaaagggccacatggcgctgcaggcccgcagcctggcgatgacggtgggcgccaccggtaaggaagtggaagccgtcgcgcagcagctcaagcgtcaaaagacgatgaaccaagaccgcgccatggccatcctgaacgatctgcgcaagcagtga

SEQ ID NO: 25 - DNA, 1152 bp, Enterococcus faecalis
atgaccatcggcattgacaagatttcctttttcgtcccgccgtactacatcgacatgacggccctcgccgaggcgcgcaacgtggaccccggcaagttccacatcggcatcggccaggatcagatggccgtcaacccgatctcgcaggatatcgtgacctttgccgccaacgcggccgaggccatcctgaccaaggaggacaaagaagccatcgacatggtcatcgtgggcaccgagtcgtcgatcgatgagagcaaggccgcggccgtcgtgctgcaccggctgatgggcatccaacccttcgcccgctccttcgagattaaggaagcctgctacggtgcgaccgcgggcctccagctggcgaagaaccacgtggccctgcacccggataagaaggtcctggtggtggccgcggacatcgcgaagtacggcctgaatagcggtggcgagccgacgcagggcgcgggcgcggtggccatgctggtcgcctcggagccgcgcatcctggccctcaaggaagataacgtgatgctgacgcaggacatctacgacttctggcgccccaccggccatccgtatccgatggtggacggtcccctgtccaatgaaacctacatccagtcgttcgcgcaagtctgggacgaacacaagaagcgcacgggcctcgacttcgccgactatgacgcgctggccttccacatcccgtacaccaagatgggcaagaaggccctgctcgccaagatcagcgaccagaccgaggccgaacaggaacgcatcctcgcgcgctatgaagagtcgatcgtctactcgcgtcgggtgggcaacctgtacaccggctcgctgtacctgggcctgatcagcctgctggagaacgcgacgaccctgacggcgggcaaccagatcggcctgttctcgtacggtagcggcgccgtggccgagttcttcaccggcgagctggtcgcgggctaccagaatcatctgcaaaaggaaacccatctggcgctgctggacaaccgcaccgaactgagcatcgccgagtacgaagccatgttcgccgaaaccctggacaccgacatcgaccagaccctggaagatgagctgaagtatagcatctccgcgatcaacaatacggtgcgcagctatcgcaactga

SEQ ID NO: 26 - DNA, 879 bp, Streptococcus pneumoniae
atgaccaagaaggtcggcgtgggccaggcccacagcaagatcattctgatcggcgagcacgccgtggtgtatggctacccggccatcagcctgccgctgctggaagtcgaagtgacgtgcaaggtggtgccggccgaatccccgtggcgtctgtatgaagaggacacgctgtcgatggccgtgtatgccagcctggagtacctgaacatcaccgaggcctgcatccgctgcgagatcgactccgcgatcccggagaagcgcggcatgggcagctcggccgccatctccatcgccgcgatccgcgccgtgttcgactactaccaagcggatctgccgcatgacgtgctggagatcctggtgaaccgggccgaaatgatcgcccacatgaatccgagcggtctggatgccaagacgtgcctgtccgaccagccgatccgtttcatcaagaacgtgggtttcaccgagctggagatggatctgagcgcgtacctggtgatcgcggacaccggcgtgtacggccacacccgcgaggccatccaggtcgtgcaaaataagggtaaggacgccctgccctttctgcacgccctgggcgaactcacccagcaggccgaagtcgcgatttcccagaaggacgccgagggcctgggtcaaatcctgagccaggcgcatctgcacctgaaggagatcggcgtgagcagcccggaagcggacttcctggtcgaaaccaccctgtcgcacggtgccctgggcgcgaagatgtcgggcggcggcctgggcggctgcatcatcgcgctggtcaccaacctgacccatgcgcaagagctggccgagcgcctggaagaaaagggcgccgtccagacgtggatcgaatcgctctga

SEQ ID NO: 27 - DNA, 1008 bp, Streptococcus pneumoniae
atgatcgccgtcaagacgtgcggcaagctgtactgggcgggcgagtatgccatcctcgaacccggccagctggccctgatcaaggacatcccgatctatatgcgtgccgaaatcgcgttcagcgattcgtaccgcatctattcggatatgttcgacttcgccgtcgatctgcgccccaatcccgactactcgctgatccaagaaaccatcgcgctcatgggcgacttcctcgccgtccgcggtcagaatctgcgcccgttcagcctggagatttgcggcaagatggagcgcgagggtaagaagttcggcctgggctcctcgggctccgtggtggtcctggtcgtgaaggcgctgctggcgctgtacgatgtctcggtggaccaagagctgctgttcaagctgacctcggccgtgctcctgaagcgcggcgacaacggctccatgggcgacctcgcgtgcatcgtggccgaggacctggtcctgtaccagtcgtttgaccgccagaaggtggcggcgtggctggaagaagagaacctggccacggtgctggagcgtgactggggcttctccatctcccaggtgaagccgaccctggaatgcgacttcctcgtgggctggaccaaggaagtggcggtgtccagccacatggtgcaacagatcaagcagaacattaaccagaattttctgacctcgtcgaaggaaaccgtcacgagcctggtggaagccctggagcagggcaagtcggagaagatcatcgaccaggtcgaggtcgcctccaagctgctggaaggcctgtccacggatatctacacccccctgctgcgccaactgaaggaagcctcgcaggacctccagaccgtggccaagagcagcggcgccggcggcggcgactgcggcatcgccctgtccttcgacgcgcagtccaccaagaccctgaagaaccggtgggcggacctgggcatcgagctcctgtaccaagagcggatcggccacgacgacaagtcgtga

SEQ ID NO: 28 - DNA, 954 bp, Streptococcus pneumoniae
atggaccgcgaaccggtcaccgtgcgctcgtacgcgaacatcgccatcatcaagtattggggcaagaagaaggaaaaggaaatggtcccggccacctccagcatctcgctgacgctggagaatatgtacaccgaaacgaccctgtcgcccctgcccgcgaacgtcaccgcggacgagttctatatcaacggccagctgcagaacgaagtggagcatgcgaagatgagcaagattatcgatcggtaccgcccggccggcgagggctttgtgcgcatcgacacgcagaataacatgccgacggccgcgggcctgagcagcagctcgtcgggcctctccgccctggtcaaggcctgcaacgcctacttcaagctgggcctggaccgctcgcagctcgcgcaagaagccaagtttgccagcggctcgtcctcccgcagcttttacggcccgctgggcgcgtgggacaaggactcgggcgaaatctacccggtggaaacggacctcaagctggccatgatcatgctggtcctggaagataagaagaagccgatctccagccgcgacggcatgaagctgtgcgtcgaaaccagcaccacgttcgatgactgggtgcggcagagcgaaaaggactaccaagacatgctgatttacctgaaggaaaacgacttcgcgaagatcggcgaactgaccgagaagaatgcgctggcgatgcacgcgacgaccaagaccgcctcgcccgccttctcgtacctgaccgacgccagctacgaagccatggccttcgtgcgccaactccgcgaaaagggcgaggcgtgctacttcacgatggacgccggcccgaacgtcaaggtgttctgccaggaaaaggatctggaacatctgtccgaaatcttcggccaccgctaccgcctgatcgtgagcaagaccaaggatctgtcgcaagacgactgctgctga

SEQ ID NO: 29 - DNA, 1011 bp, Streptococcus pneumoniae
atgacgaccaaccgcaaggatgagcacatcctctacgccctggagcagaagtcgtcgtacaactcgttcgacgaagtggaactgatccactcgtcgctgccgctgtataacctggacgaaatcgacctgtccaccgagttcgccggccgcaagtgggatttcccgttctacatcaatgccatgaccggcggtagcaacaagggccgcgaaatcaatcagaagctggcccaggtcgccgagtcgtgcggcatcctgttcgtcaccggcagctactccgccgcgctgaagaacccgaccgacgactcgttctcggtcaagagcagccacccgaatctgctgctgggcacgaacatcggcctcgacaagcccgtcgaactgggcctgcagaccgtggaagaaatgaaccccgtgctgctccaggtgcatgtgaacgtgatgcaagagctgctgatgccggagggcgaacgcaagttccgcagctggcagtcgcacctggccgactactcgaagcagatccccgtgccgatcgtgctgaaagaagtgggcttcggcatggacgccaagaccatcgagcgtgcctacgagttcggcgtgcgcaccgtggacctctcgggccgcggtggcacgagcttcgcgtacatcgaaaaccggcgcagcggccagcgcgactacctgaaccagtggggccaatcgaccatgcaggccctgctgaacgcgcaagaatggaaggacaaggtcgagctgctggtgtcgggcggcgtgcgtaacccgctcgacatgatcaagtgcctggtgttcggcgccaaggccgtgggcctgtcccgcaccgtgctggagctggtcgaaacctacaccgtcgaagaagtcatcggcattgtccagggctggaaggccgacctccgcctcatcatgtgctccctgaactgcgccacgatcgcggacctccagaaggtggactatctcctctacggcaagctcaaagaagccaaggaccagatgaagaaggcgtga

SEQ ID NO: 30 - DNA, 1683 bp, Populus alba
atgcggtgctccgtcagcaccgagaacgtgtcgttcaccgaaaccgaaacggaagcccgccgctcggcgaactacgagccgaacagctgggactacgactatctgctgtcgagcgataccgacgagagcatcgaagtgtacaaggataaggccaagaagctggaagccgaggtgcgccgcgagatcaataacgaaaaggccgagttcctgaccctgctggaactgattgacaacgtgcaacgcctgggcctgggctaccgcttcgaaagcgatatccggggcgccctggaccgtttcgtgagctccggcggtttcgacgcggtgaccaagacctccctgcacggcaccgcgctgtcgttccgtctgctgcggcagcacggcttcgaggtgtcgcaagaagccttcagcggcttcaaggaccagaacggcaacttcctggagaacctcaaggaggacatcaaggccatcctgagcctgtacgaagcctccttcctggccctggaaggcgagaacatcctggacgaagccaaggtctttgccatttcgcacctgaaggaactgtcggaagagaagatcggcaaggaactcgccgaacaggtcaaccatgcgctggagctcccgctgcaccgccggacgcagcgcctggaagccgtgtggagcatcgaggcgtaccgcaagaaggaagatgcgaaccaagtgctgctggagctggccatcctggactataacatgatccagagcgtctaccagcgtgatctgcgcgaaacgtcccgttggtggcgccgcgtcggtctggccacgaagctgcacttcgcccgcgaccgcctgatcgagtcgttctactgggccgtcggcgtcgcgtttgagccgcagtactcggactgccgcaacagcgtcgccaagatgttctcgttcgtgaccatcatcgacgacatctacgacgtgtacggcacgctggacgaactggagctgttcacggacgccgtggagcgctgggacgtgaacgcgatcaatgatctgccggactacatgaagctctgcttcctcgccctgtacaataccatcaacgaaatcgcctatgacaatctgaaggacaagggcgagaatatcctgccctacctgaccaaggcctgggcggatctctgcaacgcgtttctgcaagaagcgaagtggctgtacaacaagtccaccccgacgttcgacgactatttcggcaacgcgtggaagtcgagctcgggtccgctgcagctggtgttcgcgtacttcgcggtcgtccagaacatcaagaaagaagagatcgagaacctccagaagtatcatgacaccatctcccgcccgagccacattttccgcctctgcaacgacctggccagcgcgtcggcggagatcgcccgcggcgaaaccgcgaactcggtgtcctgctacatgcgcaccaagggcatcagcgaggaactggccacggagtcggtgatgaacctgattgacgaaacctggaagaagatgaacaaggaaaagctgggcggcagcctctttgccaagcccttcgtggaaacggcgatcaatctcgcccggcagtcgcattgcacctaccacaacggcgacgcgcacaccagccccgatgagctgacccgcaagcgcgtcctgtcggtcatcacggagccgatcctgcccttcgagcgctga

SEQ ID NO: 31 - protein, 6 aa, Artificial Sequence
  Description of Artificial Sequence Synthetic 6xHis tag
HHHHHH

(19) United States
(12) Patent Application Publication
(10) Pub. No.: US 2018/0291401 A1
(43) Pub. Date: Oct. 11, 2018

(54) METHODS, HOSTS, AND REAGENTS RELATED THERETO FOR PRODUCTION OF UNSATURATED PENTAHYDROCARBONS, DERIVATIVES AND INTERMEDIATES THEREOF

(71) Applicant: INVISTA NORTH AMERICA S.A.R.L., Wilmington, DE (US)

(72) Inventor: Alex Van Eck CONRADIE, Eaglescliffe (GB)

(21) Appl. No.: 15/932,189

(22) PCT Filed: Aug. 16, 2016
(86) PCT No.: PCT/IB2016/001233

§ 371 Date: Feb. 16, 2018

Related U.S. Application Data
(60) Provisional application No. 62/205,914, filed on Aug. 17, 2015.

(51) Int. Cl. C12P 5/00; C12P 7/42; C12N 15/52; C12N 9/04

(52) U.S. Cl. CPC C12P 5/007; C12P 7/42; C12N 15/52; C12N 9/0006; C12Y 101/01034; C12Y 401/01033; C12Y 203/0301; C12Y 503/03002; C12Y 402/03027; C12Y 207/04002; C12Y 203/01009; C12Y 207/01036

(57) ABSTRACT

This application describes methods, including non-naturally occurring methods, for biosynthesizing unsaturated pentahydrocarbons, such as isoprene and intermediates thereof, via the mevalonate pathway, as well as non-naturally occurring hosts for producing isoprene.

This application claims priority to U.S. Provisional Patent Application No. 62/205,914, filed Aug. 17, 2015.

### SEQUENCE LISTING

The instant application contains a Sequence Listing which has been submitted electronically in ASCII format and is hereby incorporated by reference in its entirety. Said ASCII copy, created on Aug. 15, 2016, is named 12444_0581-00000_SL.txt and is 77,498 bytes in size.

### TECHNICAL FIELD

This application relates to methods for biosynthesizing unsaturated pentahydrocarbons, such as isoprene and intermediates thereof, using one or more isolated enzymes such as one or more of an acetyl-CoA acetyltransferase, a hydroxymethylglutaryl-CoA synthase, a hydroxymethylglutaryl Co-A reductase, a mevalonate-kinase, a phosphomevalonate kinase, a diphosphomevalonate decarboxylase, an isopentenyl diphosphate isomerase, and an isoprene synthase; or using non-naturally occurring host cells expressing one or more such enzymes.

### BACKGROUND

Isoprene is an important monomer for the production of specialty elastomers including motor mounts/fittings, surgical gloves, rubber bands, golf balls and shoes. Styrene-isoprene-styrene block copolymers form a key component of hot-melt pressure-sensitive adhesive formulations and cis-poly-isoprene is utilized in the manufacture of tires (Whited et al., Industrial Biotechnology, 2010, 6(3), 152-163).

Manufacturers of rubber goods depend on either imported natural rubber from the Brazilian rubber tree or petroleum-based synthetic rubber polymers (Whited et al., 2010, supra). Given a reliance on petrochemical feedstocks and the harvesting of trees, biotechnology offers an alternative approach via biocatalysis. Biocatalysis is the use of biological catalysts, such as enzymes, to perform biochemical transformations of organic compounds.

Accordingly, against this background, it is clear that there is a need for sustainable methods for producing intermediates, in particular isoprene, wherein the methods are biocatalysis based.

Both bioderived feedstocks and petrochemical feedstocks are viable starting materials for the biocatalysis processes. The introduction of vinyl groups into medium carbon chain length enzyme substrates is a key consideration in synthesizing isoprene via biocatalysis processes.

There are known metabolic pathways leading to the synthesis of isoprene in prokaryotes such as Bacillus subtillis and eukaryotes such as Populus alba (Whited et al., 2010, supra).

Isoprene may be synthesized via two routes leading to the precursor dimethylvinyl-PP, such as the mevalonate and the non-mevalonate pathway (Kuzuyama, Biosci. Biotechnol. Biochem., 2002, 66(8), 1619-1627).

The mevalonate pathway incorporates a decarboxylase enzyme, mevalonate diphosphate decarboxylase (hereafter MDD), that introduces the first vinyl-group into the precursors leading to isoprene. The second vinyl-group is introduced by isoprene synthase (hereafter ISPS) in the final step in synthesizing isoprene.

The mevalonate pathway (FIG. 1) has been exploited in the biocatalytic production of isoprene using E. coli as host. E. coli engineered with the mevalonate pathway requires three moles of acetyl-CoA, three moles of ATP and two moles of NAD(P)H to produce a mole of isoprene. Given a theoretical maximum yield of 25.2% (w/w) for the mevalonate pathway, isoprene has been produced biocatalytically at a volumetric productivity of 2 g/(L·h) with a yield of 11% (w/w) from glucose (Whited et al., 2010, supra). Particularly, the phosphate activation of mevalonate to 5-diphosphomevalonate is energy intensive metabolically, requiring two moles of ATP per mole of isoprene synthesis (FIG. 1). Accordingly, reducing the ATP consumption can improve the efficiency of the pathway.

### SUMMARY

The inventors have determined that it is possible to biosynthesize unsaturated pentahydrocarbons, such as isoprene and intermediates thereof, using one or more isolated enzymes such as one or more of an acetyl-CoA acetyltransferase, a hydroxymethylglutaryl-CoA synthase, a hydroxymethylglutaryl Co-A reductase, a mevalonate-kinase, a phosphomevalonate kinase, a diphosphomevalonate decarboxylase, an isopentenyl diphosphate isomerase, and an isoprene synthase; or using non-naturally occurring host cells expressing one or more such enzymes.

In one embodiment, are methods, including non-naturally occurring methods, for synthesizing isoprene via the mevalonate pathway, comprising enzymatically converting acetyl-CoA to acetoacetyl-CoA using an acetyl-CoA acetyltransferase enzyme, for example an acetyl-CoA acetyltransferase enzyme having the amino acid sequence set forth in SEQ ID No: 1 or a functional fragment thereof; enzymatically converting acetoacetyl-CoA to 3-hydroxy-3-methylglutaryl-CoA using a hydroxymethylglutaryl-CoA synthase enzyme, for example a hydroxymethylglutaryl-CoA synthase enzyme having the amino acid sequence set forth in SEQ ID No: 2 or a functional fragment thereof; enzymatically converting 3-hydroxy-3-methylglutaryl-CoA to (R)-mevalonate using a hydroxymethylglutaryl Co-A reductase enzyme, for example a hydroxymethylglutaryl Co-A reductase having the amino acid sequence set forth in SEQ ID No: 3 or a functional fragment thereof; enzymatically converting (R)-mevalonate to (R)-5-phosphomevalonate using a mevalonate-kinase enzyme, for example a mevalonate-kinase having the amino acid sequence set forth in SEQ ID No: 4 or a functional fragment thereof; enzymatically converting (R)-5-phosphomevalonate to (R)-5-diphosphomevalonate using a phosphomevalonate kinase enzyme, for example a phosphomevalonate kinase having the amino acid sequence set forth in SEQ ID No: 5 or a functional fragment thereof; enzymatically converting (R)-5-diphosphomevalonate to isopentenyl diphosphate using a diphosphomevalonate decarboxylase enzyme, for example a diphosphomevalonate decarboxylase having the amino acid sequence set forth in SEQ ID No: 6 or a functional fragment thereof; enzymatically converting isopentenyl diphosphate to dimethylallyl diphosphate using an isopentenyl diphosphate isomerase, for example an isopentenyl diphosphate isomerase having the amino acid sequence set forth in SEQ ID No:7 or a functional fragment thereof; and enzymatically converting dimethylallyl diphosphate to isoprene using an isoprene synthase enzyme, for example an isoprene synthase having the amino acid sequence set forth in SEQ ID No: 8 or a functional fragment thereof.

In one embodiment, are methods, including non-naturally occurring methods, for synthesizing isoprene via the mevalonate pathway, comprising enzymatically converting acetyl-CoA to acetoacetyl-CoA using an acetyl-CoA acetyltransferase enzyme, for example an acetyl-CoA acetyltransferase enzyme having the amino acid sequence set forth in SEQ ID No: 9 or a functional fragment thereof; enzymatically converting acetoacetyl-CoA to 3-hydroxy-3-methylglutaryl-CoA using a hydroxymethylglutaryl-CoA synthase enzyme, for example a hydroxymethylglutaryl-CoA synthase enzyme having the amino acid sequence set forth in SEQ ID No: 10 or a functional fragment thereof; enzymatically converting 3-hydroxy-3-methylglutaryl-CoA to (R)-mevalonate using a hydroxymethylglutaryl Co-A reductase enzyme, for example a hydroxymethylglutaryl Co-A reductase having the amino acid sequence set forth in SEQ ID No: 9 or a functional fragment thereof; enzymatically converting (R)-mevalonate to (R)-5-phosphomevalonate using a mevalonate-kinase enzyme, for example a mevalonate-kinase having the amino acid sequence set forth in SEQ ID No: 11 or a functional fragment thereof; enzymatically converting (R)-5-phosphomevalonate to (R)-5-diphosphomevalonate using a phosphomevalonate kinase enzyme, for example a phosphomevalonate kinase having the amino acid sequence set forth in SEQ ID No: 12 or a functional fragment thereof; enzymatically converting (R)-5-diphosphomevalonate to isopentenyl diphosphate using a diphosphomevalonate decarboxylase enzyme, for example a diphosphomevalonate decarboxylase having the amino acid sequence set forth in SEQ ID No: 13 or a functional fragment thereof; enzymatically converting isopentenyl diphosphate to dimethylallyl diphosphate using an isopentenyl diphosphate isomerase, for example an isopentenyl diphosphate isomerase having the amino acid sequence set forth in SEQ ID No: 14 or a functional fragment thereof; and enzymatically converting dimethylallyl diphosphate to isoprene using an isoprene synthase enzyme, for example an isoprene synthase having the amino acid sequence set forth in SEQ ID No: 15 or a functional fragment thereof.

In one embodiment, the methods for synthesizing isoprene via the mevalonate pathway are performed in a non-naturally occurring host, which may be a prokaryotic or eukaryotic host. In one embodiment, the host may be a chemolithotrophic host. In one embodiment, the host may be Cupriavidus necator.

In one embodiment, at least one of the enzymatic conversions within the methods for synthesizing isoprene via the mevalonate pathway is performed in a non-naturally occurring host, which may be a prokaryotic or eukaryotic host. In one embodiment, the host may be a chemolithotrophic host. In one embodiment, the host may be Cupriavidus necator.

In one embodiment, are non-naturally occurring hosts capable of producing isoprene and/or intermediates thereof via the mevalonate pathway, said host comprising at least one exogenous nucleic acid encoding a polypeptide having the sequence set forth in SEQ ID No: 1 or a functional fragment thereof; at least one exogenous nucleic acid encoding a polypeptide having the sequence set forth in SEQ ID No: 2 or a functional fragment thereof; at least one exogenous nucleic acid encoding a polypeptide having the sequence set forth in SEQ ID No: 3 or a functional fragment thereof; at least one exogenous nucleic acid encoding a polypeptide having the sequence set forth in SEQ ID No: 4 or a functional fragment thereof; at least one exogenous nucleic acid encoding a polypeptide having the sequence set forth in SEQ ID No: 5 or a functional fragment thereof; at least one exogenous nucleic acid encoding a polypeptide having the sequence set forth in SEQ ID No: 6 or a functional fragment thereof; at least one exogenous nucleic acid encoding a polypeptide having the sequence set forth in SEQ ID No: 7 or a functional fragment thereof; at least one exogenous nucleic acid encoding a polypeptide having the sequence set forth in SEQ ID No: 8 or a functional fragment thereof; at least one exogenous nucleic acid encoding a polypeptide having the sequence set forth in SEQ ID No: 9 or a functional fragment thereof; at least one exogenous nucleic acid encoding a polypeptide having the sequence set forth in SEQ ID No:10 or a functional fragment thereof; at least one exogenous nucleic acid encoding a polypeptide having the sequence set forth in SEQ ID No: 11 or a functional fragment thereof; at least one exogenous nucleic acid encoding a polypeptide having the sequence set forth in SEQ ID No: 12 or a functional fragment thereof; at least one exogenous nucleic acid encoding a polypeptide having the sequence set forth in SEQ ID No: 13 or a functional fragment thereof; at least one exogenous nucleic acid encoding a polypeptide having the sequence set forth in SEQ ID No: 14 or a functional fragment thereof; and at least one exogenous nucleic acid encoding a polypeptide having the sequence set forth in SEQ ID No: 15 or a functional fragment thereof.

In one embodiment, hosts may be capable of endogenously producing isoprene via a non-mevalonate pathway.

In one embodiment, are methods for enzymatically converting isopentenyl diphosphate to dimethylallyl diphosphate using an isopentenyl diphosphate isomerase having the amino acid sequence set forth in SEQ ID No: 7 or SEQ ID No: 14. In one embodiment, at least one of the enzymatic conversions of the methods comprises gas fermentation, for example fermentation of at least one of natural gas, syngas, CO₂/H₂, methanol, ethanol, non-volatile residue, caustic wash from cyclohexane oxidation processes, or waste stream from a chemical or petrochemical industry.

In one embodiment, are non-naturally occurring mutants or variants of SEQ ID No: 22 or 29 comprising one or more non-naturally-occurring mutations, wherein the mutant or variant exhibits isopentenyl diphosphate isomerase activity.

Methods described herein can be performed using isolated enzymes.

Methods described herein can be performed using cell lysates comprising the enzymes.

Methods described herein can be performed in a non-naturally occurring host, such as a recombinant host. For example, the host can be a prokaryote selected from the group consisting of the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens or Pseudomonas putida; from the genus Bacillus such as Bacillus subtillis; or from the genus Rhodococcus such as Rhodococcus equi. The host can be a eukaryote, for example a eukaryote selected from the group consisting of the genus Aspergillus such as Aspergillus niger, from the genus Saccharomyces such as Saccharomyces cerevisiae; from the genus Pichia such as Pichia pastoris; from the genus Yarrowia such as Yarrowia lipolytica; from the genus Issatchenkia such as Issatchenkia orientalis; from the genus Debaryomyces such as Debaryomyces hansenii; from the genus Arxula such as Arxula adeninivorans; or from the genus Kluyveromyces such as Kluyveromyces lactis. The host can be a prokaryotic or eukaryotic chemolithotroph.

The host can be subjected to a fermentation strategy entailing anaerobic, micro-aerobic or aerobic cultivation. A cell retention strategy using a ceramic hollow fiber membrane can be employed to achieve and maintain a high cell density during fermentation.

The principal carbon source fed to the fermentation can derive from a biological or a non-biological feedstock. The biological feedstock can be, or can derive from, monosaccharides, disaccharides, hemicellulose such as levulinic acid and furfural, cellulose, lignocellulose, lignin, triglycerides such as glycerol and fatty acids, agricultural waste or municipal waste. The non-biological feedstock can be, or can derive from, either natural gas, syngas, CO₂/H₂, methanol, ethanol, non-volatile residue (NVR), caustic wash from cyclohexane oxidation processes or other waste stream from either the chemical or petrochemical industries.

The reactions of the pathways described herein can be performed in one or more cell (e.g., host cell) strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from any of the above types of host cells and used in a purified or semi-purified form. Extracted enzymes can optionally be immobilized to a solid substrate such as the floors and/or walls of appropriate reaction vessels. Moreover, such extracts include lysates (e.g., cell lysates) that can be used as sources of relevant enzymes. In the methods provided by the document, all the steps can be performed in cells (e.g., host cells), all the steps can be performed using extracted enzymes, or some of the steps can be performed in cells and others can be performed using extracted enzymes.

Unless otherwise defined, all technical and scientific terms used herein have the same meaning as commonly understood by one of ordinary skill in the art to which this application pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features, objects, and advantages will be apparent from the description and the drawings, and from the claims. The word “comprising” in the claims may be replaced by “consisting essentially of” or with “consisting of,” according to standard practice in patent law.

### DESCRIPTION OF DRAWINGS

FIG. 1 is a schematic of an exemplary biochemical pathway leading to isoprene using (R)-mevalonate as a central precursor via isopentenyl diphosphate and dimethylallyl diphosphate.

FIG. 2 is a plasmid map of pBBR1-1A-Pbad-441-442-443.

FIG. 3A is a plasmid map for pMOL28Tet-11p encoding the S. pneumoniae lower MVA pathway and FIG. 3B is a plasmid map for pISP401 encoding the P. alba isoprene synthase.

FIG. 4 contains the amino acid sequences of enzymes which may be used for biosynthesizing isoprene via the mevalonate pathway.

FIG. 5 contains nucleic acid sequences encoding enzymes which may be used for biosynthesizing isoprene via the mevalonate pathway.

FIG. 6 is a table showing mevalonolactone production in Cupriavidus necator.

FIG. 7 is a graph showing isoprene production in Cupriavidus necator.

### DETAILED DESCRIPTION

In one aspect are provided enzymes and non-naturally occurring, for example recombinant, host microorganisms for synthesis of isoprene and/or intermediates thereof in one or more enzymatic steps. In one aspect are provided enzymes and non-naturally occurring, for example recombinant, host microorganisms for synthesis of isoprene and/or intermediates thereof via the mevalonate pathway.

In one aspect are provided enzymes and non naturally occurring, for example recombinant, host microorganisms for synthesis of isoprene and/or intermediates thereof in one or more enzymatic steps comprising use of one or more of an acetyl-CoA acetyltransferase, a hydroxymethylglutaryl-CoA synthase, a hydroxymethylglutaryl Co-A reductase, a mevalonate-kinase, a phosphomevalonate kinase, a diphosphomevalonate decarboxylase, an isopentenyl diphosphate isomerase, and an isoprene synthase; or using non-naturally occurring host cells expressing one or more such enzymes.

One of skill in the art understands that compounds containing amine groups (including, but not limited to, organic amines, aminoacids, and diamines) are formed or converted to their ionic salt form, for example, by addition of an acidic proton to the amine to form the ammonium salt, formed with inorganic acids such as hydrochloric acid, hydrobromic acid, sulfuric acid, nitric acid, phosphoric acid, and the like; or formed with organic acids including, but not limited to, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include, but are not limited to, aluminum hydroxide, calcium hydroxide, potassium hydroxide, sodium carbonate, sodium hydroxide, and the like. A salt of the present invention is isolated as a salt or converted to the free amine by raising the pH to above the pKb through addition of base or treatment with a basic ion exchange resin.

One of skill in the art understands that compounds containing both amine groups and carboxylic acid groups (including, but not limited to, aminoacids) are formed or converted to their ionic salt form by either 1) acid addition salts, formed with inorganic acids including, but not limited to, hydrochloric acid, hydrobromic acid, sulfuric acid, nitric acid, phosphoric acid, and the like; or formed with organic acids including, but not limited to, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include, but are not limited to, aluminum hydroxide, calcium hydroxide, potassium hydroxide, sodium carbonate, sodium hydroxide; and the like, or 2) when an acidic proton present in the parent compound either is replaced by a metal ion, e.g., an alkali metal ion, an alkaline earth ion, or an aluminum ion; or coordinates with an organic base. Acceptable organic bases include, but are not limited to, ethanolamine, diethanolamine, triethanolamine, tromethamine, N-methylglucamine, and the like. Acceptable inorganic bases include, but are not limited to, aluminum hydroxide, calcium hydroxide, potassium hydroxide, sodium carbonate, sodium hydroxide, and the like. A salt can of the present invention is isolated as a salt or converted to the free acid by reducing the pH to below the pKa through addition of acid or treatment with an acidic ion exchange resin.

Host microorganisms described herein can include pathways that can be manipulated such that isoprene or its intermediates can be produced. In an endogenous pathway, the host microorganism naturally expresses all of the enzymes catalyzing the reactions within the pathway. A host microorganism containing an engineered pathway does not naturally express all of the enzymes catalyzing the reactions within the pathway but has been engineered such that all of the enzymes within the pathway are expressed in the host.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., gDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

For example, depending on the host and the compounds produced by the host, one or more of the following enzymes may be expressed in the host: an acetyl-CoA acetyltransferase, a hydroxymethylglutaryl-CoA synthase, a hydroxymethylglutaryl Co-A reductase, a mevalonate-kinase, a phosphomevalonate kinase, a diphosphomevalonate decarboxylase, an isopentenyl diphosphate isomerase, and an isoprene synthase.

As used herein, the term “mevalonate pathway” refers to the production of isoprene by enzymatically converting acetyl-CoA to acetoacetyl-CoA using an acetyl-CoA acetyltransferase enzyme; enzymatically converting acetoacetyl-CoA to 3-hydroxy-3-methylglutaryl-CoA using a hydroxymethylglutaryl-CoA synthase enzyme; enzymatically converting 3-hydroxy-3-methylglutaryl-CoA to (R)-mevalonate using a hydroxymethylglutaryl Co-A reductase; enzymatically converting (R)-mevalonate to (R)-5-phosphomevalonate using a mevalonate-kinase enzyme; enzymatically converting (R)-5-phosphomevalonate to (R)-5-diphosphomevalonate using a phosphomevalonate kinase enzyme; enzymatically converting (R)-5-diphosphomevalonate to isopentenyl diphosphate using a diphosphomevalonate decarboxylase enzyme; enzymatically converting isopentenyl diphosphate to dimethylallyl diphosphate using an isopentenyl diphosphate isomerase; and enzymatically converting dimethylallyl diphosphate to isoprene using a polypeptide having isoprene synthase enzyme.

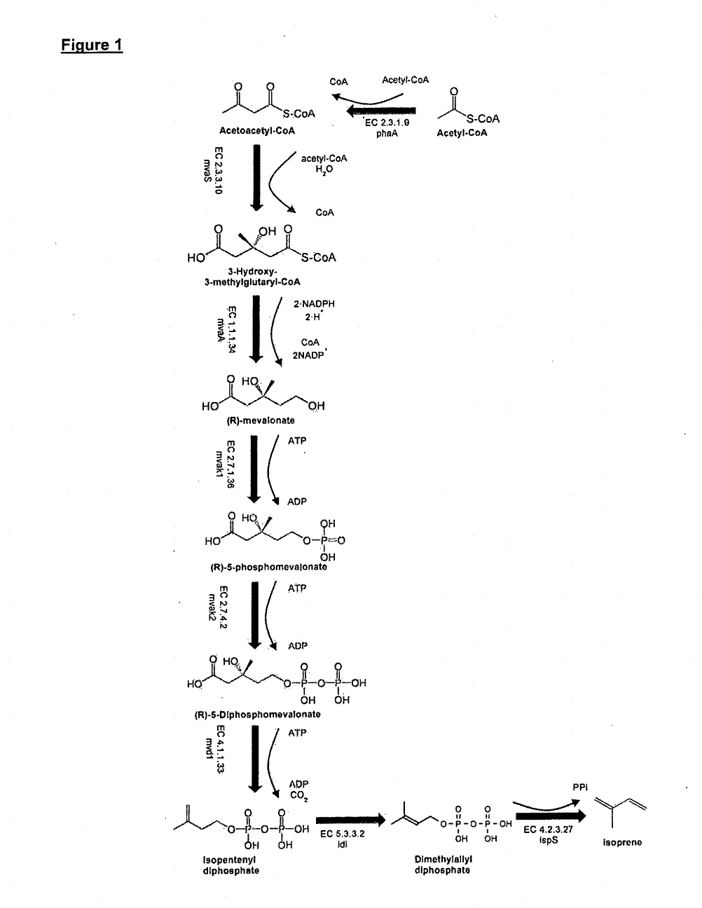

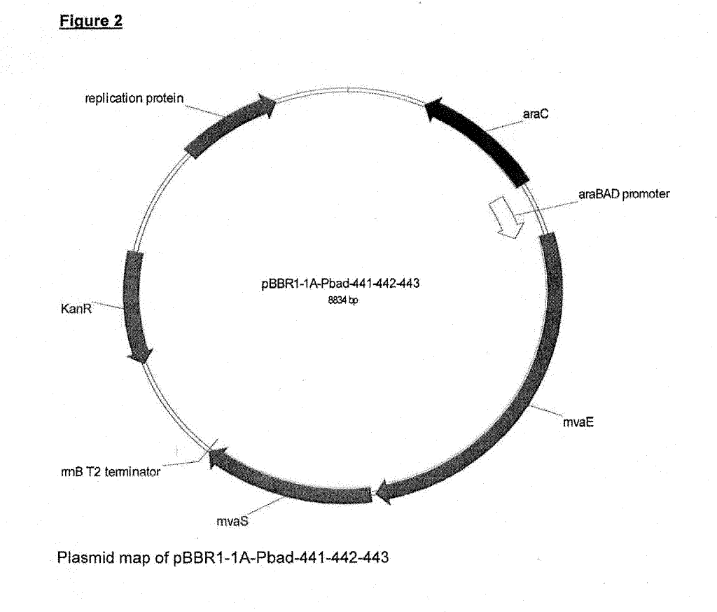

In one embodiment are provided means for producing isoprene. In one embodiment, the structures that may be used to produce isoprene are the enzymes identified in FIG. 1.

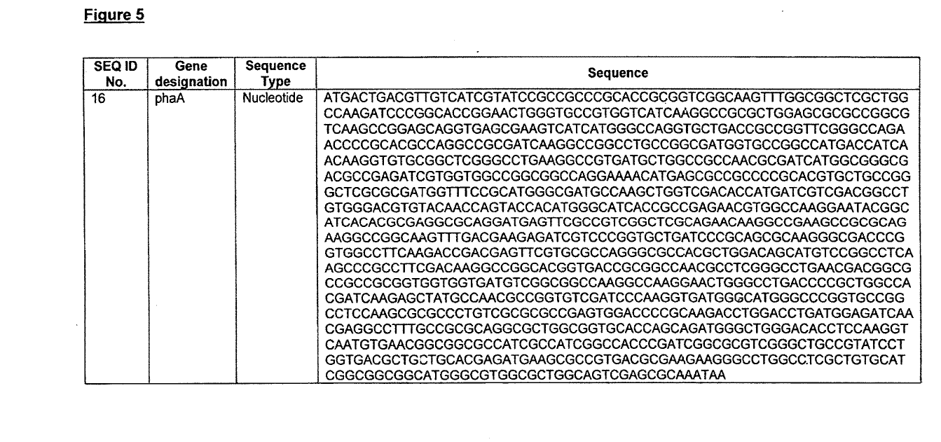

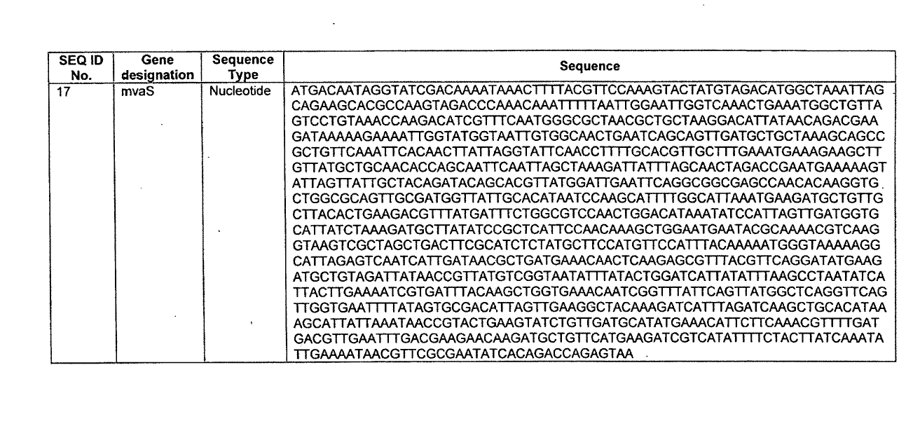

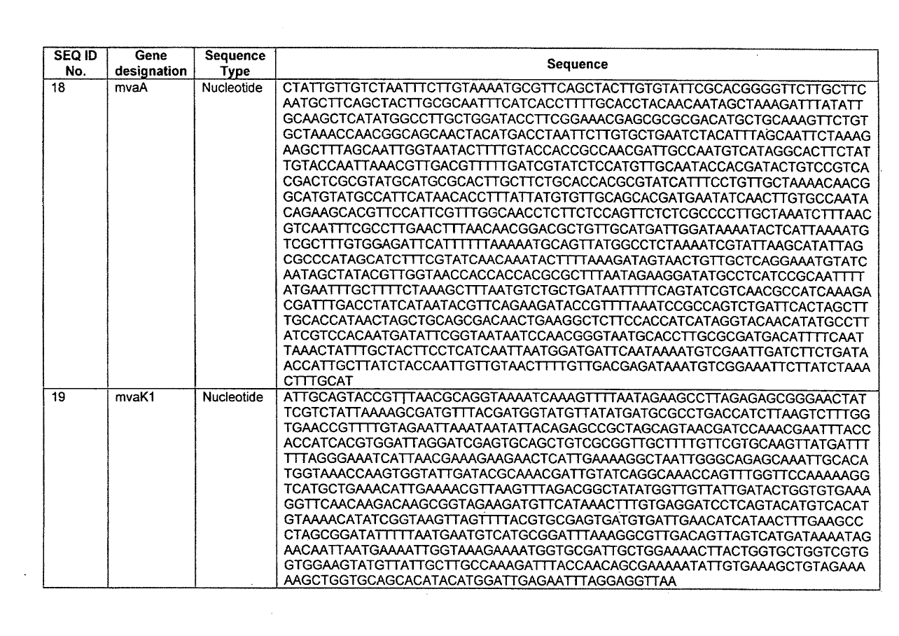

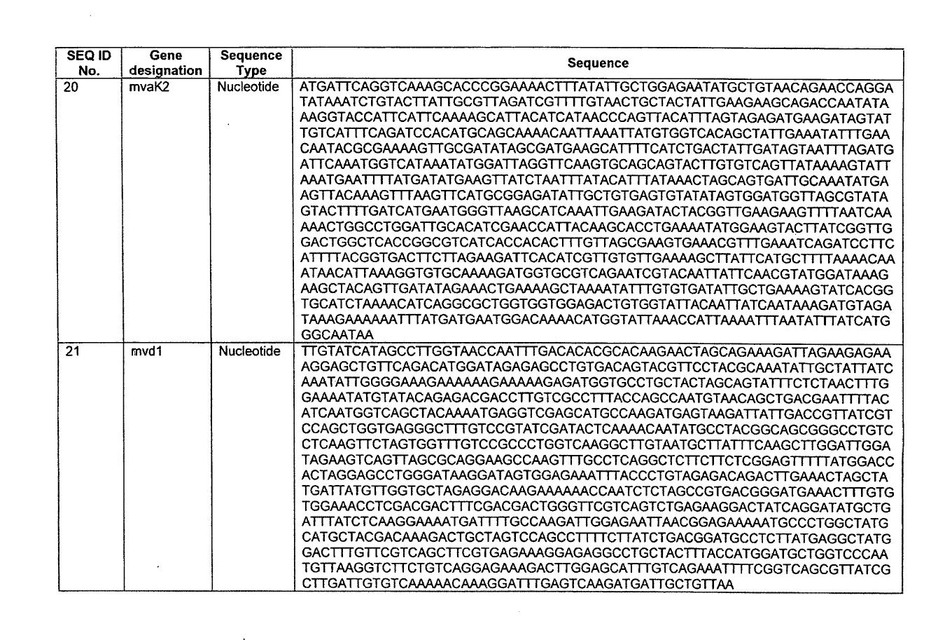

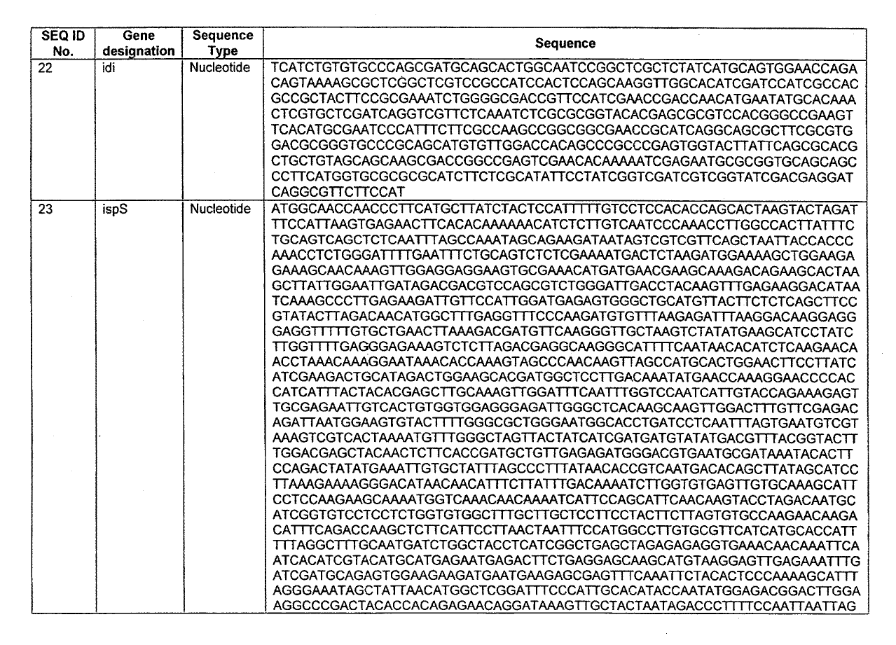

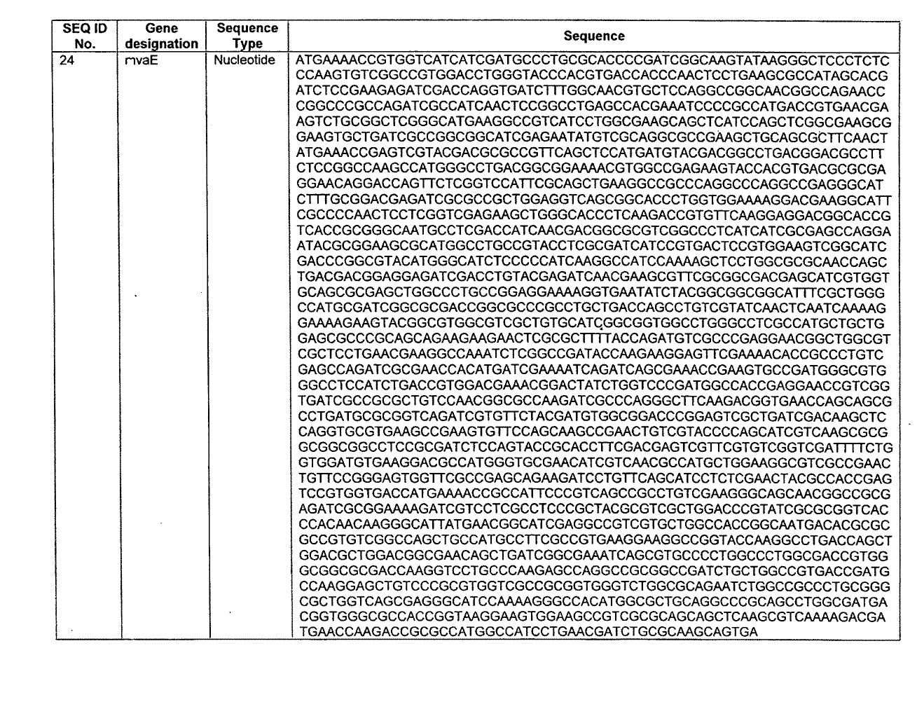

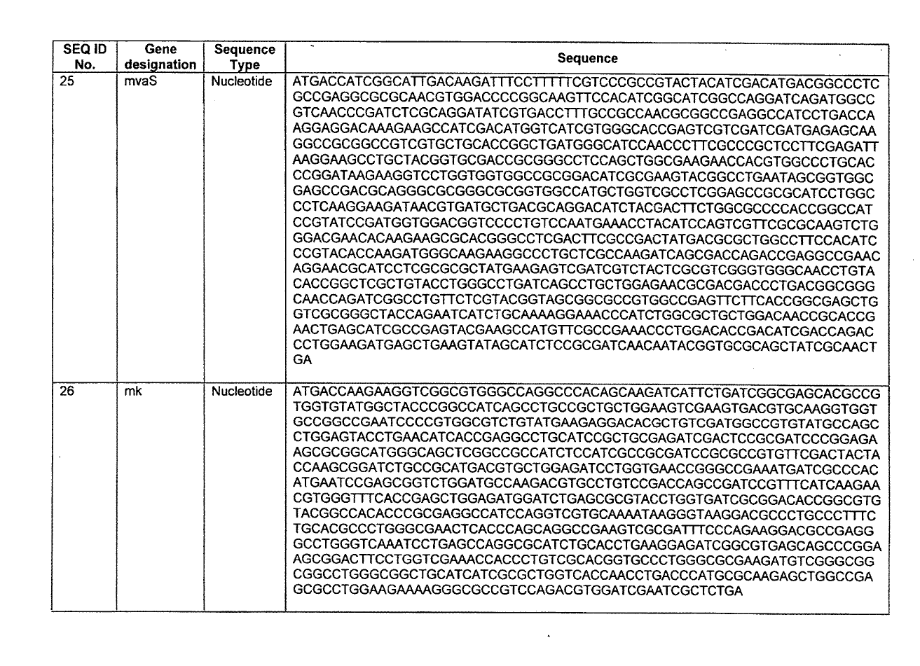

In one embodiment the acetyl-CoA acetyltransferase is the gene product of phaA. In one embodiment the acetyl-CoA acetyltransferase is classified under EC 2.3.1.9. In one embodiment the acetyl-CoA acetyltransferase is a Cupriavidus necator acetyl-CoA acetyltransferase (Genbank Accession No. AAA21972.1, SEQ ID No: 1). See FIG. 4. In one embodiment the acetyl-CoA acetyltransferase is a Cupriavidus necator acetyl-CoA acetyltransferase encoded by a nucleic acid having the sequence set forth in SEQ ID No: 16. See FIG. 5.

In one embodiment the hydroxymethylglutaryl-CoA synthase is the gene product of mvaS. In one embodiment the hydroxymethylglutaryl-CoA synthase is classified under EC 2.3.3.10. In one embodiment the hydroxymethylglutaryl-CoA synthase is a Staphylococcus aureus hydroxymethylglutaryl-CoA synthase (Genbank Accession No. BAB58708.1, SEQ ID No: 2). See FIG. 4. In one embodiment the hydroxymethylglutaryl-CoA synthase is a Staphylococcus aureus hydroxymethylglutaryl-CoA synthase encoded by a nucleic acid having the sequence set forth in SEQ ID No: 17. See FIG. 5.

In one embodiment the hydroxymethylglutaryl Co-A reductase is the gene product of mvaA. In one embodiment the hydroxymethylglutaryl Co-A reductase is classified under EC 1.1.1.34. In one embodiment the hydroxymethylglutaryl Co-A reductase is a Staphylococcus aureus hydroxymethylglutaryl Co-A reductase (Genbank Accession No. BAB58707.1, SEQ ID No: 3). See FIG. 4. In one embodiment the hydroxymethylglutaryl Co-A reductase is a Staphylococcus aureus hydroxymethylglutaryl Co-A reductase encoded by a nucleic acid having the sequence set forth in SEQ ID No: 18. See FIG. 5.

In one embodiment the mevalonate-kinase is the gene product of mvak1. In one embodiment the mevalonate-kinase is classified under EC 2.7.1.36. In one embodiment the mevalonate-kinase is a Staphylococcus aureus mevalonate-kinase (Genbank Accession No. BAB56752.1, SEQ ID No: 4). See FIG. 4. In one embodiment the mevalonate-kinase is a Staphylococcus aureus mevalonate-kinase encoded by a nucleic acid having the sequence set forth in SEQ ID No: 19. See FIG. 5.

In one embodiment the phosphomevalonate kinase is the gene product of mvak2. In one embodiment the phosphomevalonate kinase is classified under EC 2.7.4.2. In one embodiment the phosphomevalonate kinase is a Staphylococcus aureus phosphomevalonate kinase (Genbank Accession No. 6AB56754.1, SEQ ID No: 5). See FIG. 4. In one embodiment the phosphomevalonate kinase is a Staphylococcus aureus phosphomevalonate kinase encoded by a nucleic acid having the sequence set forth in SEQ ID No: 20. See FIG. 5.

In one embodiment the diphosphomevalonate decarboxylase is the gene product of mvd1. In one embodiment the diphosphomevalonate decarboxylase is classified under EC 4.1.1.33. In one embodiment the diphosphomevalonate decarboxylase is a Streptococcus pneumoniae diphosphomevalonate decarboxylase (Genbank Accession No. AAK99143.1, SEQ ID No: 6). See FIG. 4. In one embodiment the diphosphomevalonate decarboxylase is a Streptococcus pneumoniae diphosphomevalonate decarboxylase encoded by a nucleic acid having the sequence set forth in SEQ ID No: 21. See FIG. 5.

In one embodiment the isopentenyl diphosphate isomerase is the gene product of idi. In one embodiment the isopentenyl diphosphate isomerase is classified under EC 5.3.3.2. In one embodiment the isopentenyl diphosphate isomerase is a Burkholderia multivorans isopentenyl diphosphate isomerase (Genbank Accession No. ABX19602.1, SEQ ID No: 7). See FIG. 4. In one embodiment the isopentenyl diphosphate isomerase is a Burkholderia multivorans isopentenyl diphosphate isomerase encoded by a nucleic acid having the sequence set forth in SEQ ID No: 22. See FIG. 5.

In one embodiment the isoprene synthase is the gene product of ispS. In one embodiment the isoprene synthase is classified under EC 4.2.3.27. In one embodiment the isoprene synthase is a Mucuna pruriens isoprene synthase (SEQ ID No: 8). See FIG. 4. In one embodiment the isoprene synthase is a Mucuna pruriens isoprene synthase encoded by a nucleic acid having the sequence set forth in SEQ ID No: 23. See FIG. 5.

In one embodiment the gene product of mvaE has dual acetoacetyl-CoA C-acetyltransferase and HMG-CoA reductase activity. In one embodiment the acetyl-CoA acetyltransferase is classified under EC 2.3.1.9. In one embodiment the enzyme with dual acetoacetyl-CoA C-acetyltransferase and HMG-CoA reductase activity is from Enterococcus faecalis (Genbank Accession No. J6EWX4, SEQ ID No: 9). See FIG. 4. In one embodiment the enzyme with dual acetoacetyl-CoA C-acetyltransferase and HMG-CoA reductase activity is from Enterococcus faecalis and is encoded by a nucleic acid having the sequence set forth in SEQ ID No: 24. See FIG. 5.

In one embodiment the hydroxymethylglutaryl-CoA synthase is the gene product of mvaS. In one embodiment the hydroxymethylglutaryl-CoA synthase is classified under EC 2.3.3.10. In one embodiment the hydroxymethylglutaryl-CoA synthase is a Enterococcus faecalis hydroxymethylglutaryl-CoA synthase (Genbank Accession No. Q835L4, SEQ ID No: 10). See FIG. 4. In one embodiment the hydroxymethylglutaryl-CoA synthase is a Enterococcus faecalis hydroxymethylglutaryl-CoA synthase encoded by a nucleic acid having the sequence set forth in SEQ ID No: 25. See FIG. 5.

In one embodiment the mevalonate-kinase is the gene product of mk. In one embodiment the mevalonate-kinase is classified under EC 2.7.1.36. In one embodiment the mevalonate-kinase is a Streptococcus pneumoniae mevalonate-kinase (Accession No. WP_000163323, SEQ ID No: 11). See FIG. 4. In one embodiment the mevalonate-kinase is a Streptococcus pneumoniae mevalonate-kinase encoded by a nucleic acid having the sequence set forth in SEQ ID No: 26. See FIG. 5.

In one embodiment the phosphomevalonate kinase is the gene product of mpk. In one embodiment the phosphomevalonate kinase is classified under EC 2.7.4.2. In one embodiment the phosphomevalonate kinase is a Streptococcus pneumoniae phosphomevalonate kinase (Accession No. WP_000562415, SEQ ID No: 12). See FIG. 4. In one embodiment the phosphomevalonate kinase is a Streptococcus pneumoniae phosphomevalonate kinase encoded by a nucleic acid having the sequence set forth in SEQ ID No: 27. See FIG. 5.

In one embodiment the diphosphomevalonate decarboxylase is the gene product of mdd. In one embodiment the diphosphomevalonate decarboxylase is classified under EC 4.1.1.33. In one embodiment the diphosphomevalonate decarboxylase is a Streptococcus pneumoniae diphosphomevalonate decarboxylase (Accession No. WP_000373455, SEQ ID No: 13). See FIG. 4. In one embodiment the diphosphomevalonate decarboxylase is a Streptococcus pneumoniae diphosphomevalonate decarboxylase encoded by a nucleic acid having the sequence set forth in SEQ ID No: 28. See FIG. 5.

In one embodiment the isopentenyl diphosphate isomerase is the gene product of idi. In one embodiment the isopentenyl diphosphate isomerase is classified under EC 5.3.3.2. In one embodiment the isopentenyl diphosphate isomerase is a Streptococcus pneumoniae isopentenyl diphosphate isomerase (Accession No. WP_000210618, SEQ ID No: 14). See FIG. 4. In one embodiment the isopentenyl diphosphate isomerase is a Streptococcus pneumoniae isopentenyl diphosphate isomerase encoded by a nucleic acid having the sequence set forth in SEQ ID No: 29. See FIG. 5.

In one embodiment the isoprene synthase is the gene product of ispS. In one embodiment the isoprene synthase is classified under EC 4.2.3.27. In one embodiment the isoprene synthase is a Populus alba isoprene synthase (Accession No. Q50L36, SEQ ID No: 15). See FIG. 4. In one embodiment the isoprene synthase is a Populus alba isoprene synthase encoded by a nucleic acid having the sequence set forth in SEQ ID No: 30. See FIG. 5.

Within an engineered pathway, the enzymes can be from a single source, i.e., from one species, or can be from multiple sources, i.e., different species. Nucleic acids encoding the enzymes described herein have been identified from various organisms and are readily available in publicly available databases such as GenBank or EMBL.

Any of the enzymes described herein that can be used for isoprene production can have at least 70% sequence identity (homology) (e.g., at least 75%, 80%, 85%, 90%, 95%, 97%, 98%, 99%, or 100%) to the amino acid sequence of the corresponding wild-type enzyme.

For example, an acetyl-CoA acetyltransferase described herein can have at least 70% sequence identity (homology) (e.g., at least 75%, 80%, 85%, 90%, 95%, 97%, 98%, 99%, or 100%) to the amino acid sequence of a Cupriavidus necator acetyl-CoA acetyltransferase (Genbank Accession No. AAA21972.1, SEQ ID No: 1). See FIG. 4.

For example, a hydroxymethylglutaryl-CoA synthase described herein can have at least 70% sequence identity (homology) (e.g., at least 75%, 80%, 85%, 90%, 95%, 97%, 98%, 99%, or 100%) to the amino acid sequence of a Staphylococcus aureus hydroxymethylglutaryl-CoA synthase (Genbank Accession No. BAB58708.1, SEQ ID No: 2). See FIG. 4.

For example, a hydroxymethylglutaryl Co-A reductase described herein can have at least 70% sequence identity (homology) (e.g., at least 75%, 80%, 85%, 90%, 95%, 97%, 98%, 99%, or 100%) to the amino acid sequence of a Staphylococcus aureus hydroxymethylglutaryl Co-A reductase (Genbank Accession No. BAB58707.1, SEQ ID No: 3). See FIG. 4.

For example, a mevalonate-kinase described herein can have at least 70% sequence identity (homology) (e.g., at least 75%, 80%, 85%, 90%, 95%, 97%, 98%, 99%, or 100%) to the amino acid sequence of a Staphylococcus aureus mevalonate-kinase (Genbank Accession No. BAB56752.1, SEQ ID No: 4). See FIG. 4.

For example, a phosphomevalonate kinase described herein can have at least 70% sequence identity (homology) (e.g., at least 75%, 80%, 85%, 90%, 95%, 97%, 98%, 99%, or 100%) to the amino acid sequence of a Staphylococcus aureus phosphomevalonate kinase (Genbank Accession No. BAB56754.1, SEQ ID No: 5). See FIG. 4.

For example, a diphosphomevalonate decarboxylase described herein can have at least 70% sequence identity (homology) (e.g., at least 75%, 80%, 85%, 90%, 95%, 97%, 98%, 99%, or 100%) to the amino acid sequence of a Streptococcus pneumoniae diphosphomevalonate decarboxylase (Genbank Accession No. AAK99143.1, SEQ ID No: 6). See FIG. 4.

For example, an isopentenyl diphosphate isomerase described herein can have at least 70% sequence identity (homology) (e.g., at least 75%, 80%, 85%, 90%, 95%, 97%, 98%, 99%, or 100%) to the amino acid sequence of a Burkholderia multivorans isopentenyl diphosphate isomerase (Genbank Accession No. ABX19602.1, SEQ ID No: 7). See FIG. 4.

For example, an isoprene synthase described herein can have at least 70% sequence identity (homology) (e.g., at least 75%, 80%, 85%, 90%, 95%, 97%, 98%, 99%, or 100%) to the amino acid sequence of a Mucuna pruriens isoprene synthase (SEQ ID No: 8). See FIG. 4.

For example, an enzyme having dual acetoacetyl-CoA C acetyltransferase and HMG-CoA reductase activity described herein can have at least 70% sequence identity (homology) (e.g., at least 75%, 80%, 85%, 90%, 95%, 97%, 98%, 99%, or 100%) to the amino acid sequence of an enzyme from Enterococcus faecalis having dual acetoacetyl-CoA C acetyltransferase and HMG-CoA reductase (Genbank Accession No. J6EWX4, SEQ ID No: 9). See FIG. 4.

For example, a hydroxymethylglutaryl-CoA synthase described herein can have at least 70% sequence identity (homology) (e.g., at least 75%, 80%, 85%, 90%, 95%, 97%, 98%, 99%, or 100%) to the amino acid sequence of an Enterococcus faecalis hydroxymethylglutaryl-CoA synthase (Genbank Accession No. Q835L4, SEQ ID No: 10). See FIG. 4.

For example, a mevalonate-kinase described herein can have at least 70% sequence identity (homology) (e.g., at least 75%, 80%, 85%, 90%, 95%, 97%, 98%, 99%, or 100%) to the amino acid sequence of a Streptococcus pneumoniae mevalonate-kinase (Accession No. WP_000163323, SEQ ID No: 11). See FIG. 4.

For example, a phosphomevalonate kinase described herein can have at least 70% sequence identity (homology) (e.g., at least 75%, 80%, 85%, 90%, 95%, 97%, 98%, 99%, or 100%) to the amino acid sequence of a Streptococcus pneumoniae phosphomevalonate kinase (Accession No. WP_000562415, SEQ ID No: 12). See FIG. 4.

For example, a diphosphomevalonate decarboxylase described herein can have at least 70% sequence identity (homology) (e.g., at least 75%, 80%, 85%, 90%, 95%, 97%, 98%, 99%, or 100%) to the amino acid sequence of a Streptococcus pneumoniae diphosphomevalonate decarboxylase (Accession No. WP_000373455, SEQ ID No: 13). See FIG. 4.

For example, an isopentenyl diphosphate isomerase described herein can have at least 70% sequence identity (homology) (e.g., at least 75%, 80%, 85%, 90%, 95%, 97%, 98%, 99%, or 100%) to the amino acid sequence of a Streptococcus pneumoniae isopentenyl diphosphate isomerase (Accession No. WP_000210618, SEQ ID No: 14). See FIG. 4.

For example, an isoprene synthase described herein can have at least 70% sequence identity (homology) (e.g., at least 75%, 80%, 85%, 90%, 95%, 97%, 98%, 99%, or 100%) to the amino acid sequence of a Populus alba isoprene synthase (Accession No. Q50L36, SEQ ID No: 15). See FIG. 4.

The percent identity (homology) between two amino acid sequences can be determined by any method known to those skilled in the art. In one embodiment, the percent identity (homology) can be determined by aligning the amino acid sequences using the BLAST 2 Sequences (B 12seq) program from the stand-alone version of BLASTZ containing BLASTP version 2.0.14. This standalone version of BLASTZ can be obtained from the U.S. government's National Center for Biotechnology Information web site (www.ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B 12seq are set as follows: −i is set to a file containing the first amino acid sequence to be compared (e.g., C:\seqI.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\B12seq c:\seqI.txt −j cAseq2.txt −p blastp −o c:\output.txt. If the two compared sequences share homology (identity), then the designated output file will present those regions of homology as aligned sequences. If the two compared sequences do not share homology (identity), then the designated output file will not present aligned sequences. Similar procedures can be used for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18; and 78.19 is rounded up to 78.2. It also is noted that the length value will always be an integer.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods of the document. The term “functional fragment” as used herein refers to a peptide fragment of a protein that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, poly histidine (e.g., hexahistidine (SEQ ID NO: 31)), hemagluttanin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Hosts can naturally express none or some (e.g., one or more, two or more, three or more, four or more, five or more, or six or more) of the enzymes of the pathways described herein. Endogenous genes of the recombinant hosts also can be disrupted to prevent the formation of undesirable metabolites or prevent the loss of intermediates in the pathway through other enzymes acting on such intermediates. Recombinant hosts can be referred to as recombinant host cells, non-naturally occurring host cells, engineered cells, or engineered hosts. Thus, as described herein, recombinant hosts can include nucleic acids encoding one or more of a decarboxylase, a kinase, a dehydrogenase, a monooxygenase, an acyl [acyl carrier protein (acp)] dehydrogenase, a dehydratase, a thioesterase, or a decarboxyating thioesterase as described in more detail below.

In addition, the production of isoprene can be performed in vitro using the isolated enzymes described herein, using a lysate (e.g., a cell lysate) from a host microorganism as a source of the enzymes, or using a plurality of lysates from different host microorganisms as the source of the enzymes.

In some embodiments, the enzymes of the pathway described in FIG. 1 are the result of enzyme engineering to improve activity or specificity using the enzyme structure and wild-type residue diversity to inform the rational enzyme design.

In some embodiments, the nucleic acids encoding the enzymes of the pathway described in FIG. 1 are introduced into a host microorganism that is either a prokaryote or eukaryote.

### Cultivation Strategies

For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct recombinant host cells described herein that are capable of producing isoprene or precursors thereof.

In some embodiments, the host microorganism is a eukaryote. For example, the eukaryote can be a filamentous fungus, e.g., one from the genus Aspergillus such as Aspergillus niger. Alternatively, the eukaryote can be a yeast, e.g., one from the genus Saccharomyces such as Saccharomyces cerevisiae; from the genus Pichia such as Pichia pastoris; or from the genus Yarrowia such as Yarrowia lipolytica; from the genus Issatchenkia such as Issatchenkia orientalis; from the genus Debaryomyces such as Debaryomyces hansenii; from the genus Arxula such as Arxula adeninivorans; or from the genus Kluyveromyces such as Kluyveromyces lactis. Such eukaryotes also can be a source of genes to construct recombinant host cells described herein that are capable of producing isoprene or precursors thereof.

In some embodiments, isoprene is biosynthesized in a recombinant host using a fermentation strategy that can include anaerobic, micro-aerobic or aerobic cultivation of the recombinant host.

In some embodiments, isoprene is biosynthesized in a chemolithotrophic recombinant host.

In some embodiments, isoprene is biosynthesized in a recombinant host using a fermentation strategy that uses an alternate final electron acceptor to oxygen such as nitrate.

In some embodiments, a cell retention strategy using, for example, ceramic hollow fiber membranes can be employed to achieve and maintain a high cell density during either fed batch or continuous fermentation in the synthesis of isoprene.

In some embodiments, the biological feedstock can be, can include, or can derive from, monosaccharides, disaccharides, lignocellulose, hemicellulose, cellulose, lignin, levulinic acid & formic acid, triglycerides, glycerol, fatty acids, agricultural waste, condensed distillers' solubles, or municipal waste.

The efficient catabolism of crude glycerol stemming from the production of biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166, 1801-1813; Yang et al., Biotechnology for Biofuels, 2012, 5:13; Meijnen et al., Appl. Microbial. Biotechnol., 2011, 90, 885-893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, Journal of Biotechnology, 2011, 155, 2011, 293-298; Martin and Prather, Journal of Biotechnology, 2009, 139, 61-67).

The efficient catabolism of lignin-derived aromatic compounds such benzoate analogues has been demonstrated in several microorganisms such as Pseudomonas putida, Cupriavidus necator (Bugg et al., Current Opinion in Biotechnology, 2011, 22, 394-400; Perez-Pantoja et al, FEMS Microbial. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99(7), 2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, Journal of Biotechnology, 2003, 104, 155-172; Wee et al., Food Technol. Biotechnol., 2006, 44(2), 163-172; Ohashi et al., Journal of Bioscience and Bioengineering, 1999, 87(5), 647-654).

The efficient utilization of furfural, derived from a variety of agricultural lignocellulosic sources, has been demonstrated for Cupriavidus necator (Li et al., Biodegradation, 2011, 22, 1215-1225).

In some embodiments, the non-biological feedstock can be or can derive from natural gas, syngas, CO₂/H₂, methanol, ethanol, benzoic acid, non-volatile residue (NVR) or a caustic wash waste stream from cyclohexane oxidation processes, or terephthalic acid/isophthalic acid mixture waste streams.

The efficient catabolism of methanol has been demonstrated for the methylotropic yeast Pichia pastoris.

The efficient catabolism of ethanol has been demonstrated for Clostridium kluyveri (Seedorf et al., Proc. Natl. Acad. Sci. USA, 2008, 105(6) 2128-2133). The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Kopke et al., Applied and Environmental Microbiology, 2011, 77(15), 5467-5475).

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52(1), 152-156).

In some embodiments, substantially pure cultures of recombinant host microorganisms are provided. As used herein, a “substantially pure culture” of a recombinant host microorganism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the recombinant microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of recombinant microorganisms includes the cells and a growth, storage, or transport medium. Media can be liquid, semi-solid (e.g., gelatinous media), or frozen. The culture includes the cells growing in the liquid or inion the semi-solid medium or being stored or transported in a storage or transport medium, including a frozen storage or transport medium. The cultures are in a culture vessel or storage vessel or substrate (e.g., a culture dish, flask, or tube or a storage vial or tube).

### Metabolic Engineering

The present document provides methods involving less than or more than all the steps described for all the above pathways. Such methods can involve, for example, one, two, three, four, five, six, seven, eight, nine, ten, or more of such steps. Where less than all the steps are included in such a method, the first step can be any one of the steps listed. Furthermore, recombinant hosts described herein can include any combination of the above enzymes such that one or more of the steps, e.g., one, two, three, four, five, six, seven, eight, nine, ten, or more of such steps, can be performed within a recombinant host.

In addition, this document recognizes that where enzymes have been described as accepting CoA-activated substrates, analogous enzyme activities associated with [acp]-bound substrates exist that are not necessarily in the same enzyme class.

Also, this document recognizes that where enzymes have been described accepting (R)-enantiomers of substrate, analogous enzyme activities associated with (S)-enantiomer substrates exist that are not necessarily in the same enzyme class.

This document also recognizes that where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, this document recognizes that where enzymes have high specificity for e.g., a particular co-factor such as NADH, an enzyme with similar or identical activity that has high specificity for the co-factor NADPH may be in a different enzyme class.

In some embodiments, the enzymes in the pathways outlined herein can be the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways outlined herein can be gene dosed, i.e., overexpressed, into the resulting genetically modified organism via episomal or chromosomal integration approaches.

In some embodiments, genome-scale system biology techniques such as Flux Balance Analysis can be utilized to devise genome scale attenuation or knockout strategies for directing carbon flux to isoprene.

In some embodiments, fluxomic, metabolomic and transcriptomal data can be utilized to inform or support genome-scale system biology techniques, thereby devising genome scale attenuation or knockout strategies in directing carbon flux to isoprene.

In some embodiments, enzymes from the mevalonate pathway, for example, at least one enzyme classified under EC 2.3.1.9, EC 2.3.3.10, EC 1.1.1.34, EC 2.7.1.36, EC 2.7.4.2, EC 4.1.1.33, EC 5.3.3.2, or EC 4.2.3.27 is introduced or gene dosed into a host microorganism that utilizes the non-mevalonate or 2-C-methyl-D-erythritol 4-phosphate pathway for isoprenoid synthesis. In some embodiments, at least one enzyme having the amino acid sequence listed in SEQ ID No: 1, SEQ ID No: 2, SEQ ID No: 3, SEQ ID No: 4, SEQ ID No: 5, SEQ ID No: 6, SEQ ID No: 7, SEQ ID No: 8, SEQ ID No:9, SEQ ID No:10, SEQ ID No:11, SEQ ID No:12, SEQ ID No:13, SEQ ID No:14 or SEQ ID No:15 is introduced or gene dosed into a host microorganism that utilizes the non-mevalonate or 2-C-methyl-D-erythritol 4-phosphate pathway for isoprenoid synthesis.

In some embodiments, where pathways require excess. NADPH co-factor in the synthesis of isoprene, a puridine nucleotide transhydrogenase gene such as UdhA can be overexpressed in the host organism (Brigham et al., Advanced Biofuels and Bioproducts, 2012, Chapter 39, 1065-1090).

In some embodiments, where pathways require excess NADPH co-factor in the synthesis of isoprene, a glyceraldehyde-3P-dehydrogenase gene such as GapN can be overexpressed in the host organism (Brigham et al., 2012, supra).

In some embodiments, where pathways require excess NADPH co-factor in the synthesis of isoprene, a malic enzyme gene such as macA or maeB can be overexpressed in the host organism (Brigham et al., 2012, supra).

In some embodiments, where pathways require excess NADPH co-factor in the synthesis of isoprene, a glucose-6-phosphate dehydrogenase gene such as zwf can be overexpressed in the host organism (Lim et al., Journal of Bioscience and Bioengineering, 2002, 93(6), 543-549).

In some embodiments, where pathways require excess NADPH co-factor in the synthesis of isoprene, a fructose 1,6 diphosphatase gene such as fbp can be overexpressed in the host (Becker et al., Journal of Biotechnology, 2007, 132, 99-109).

In some embodiments, the efflux of isoprene across the cell membrane to the extracellular media can be enhanced or amplified by genetically engineering structural modifications to the cell membrane or increasing any associated transporter activity for isoprene.

### Producing Isoprene Using a Recombinant Host

Typically, isoprene is produced by providing a host microorganism and culturing the provided microorganism with a culture medium containing a suitable carbon source as described above. In general, the culture media and/or culture conditions can be such that the microorganisms grow to an adequate density and produce isoprene efficiently. For large-scale production processes, any method can be used such as those described elsewhere (Manual of Industrial Microbiology and Biotechnology, 2nd Edition, Editors: A. L. Demain and J. E. Davies, ASM Press; and Principles of Fermentation Technology, P. F. Stanbury and A. Whitaker, Pergamon). In one example, a large tank (e.g., a 100 gallon, 200 gallon, 500 gallon, or more tank) containing an appropriate culture medium is inoculated with a particular microorganism. After inoculation, the microorganism is incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank.

Once transferred, the microorganisms can be incubated to allow for the production of isoprene. In one example, a substrate comprising CO is provided to a bioreactor comprising one or more microorganisms and anaerobically fermenting the substrate to produce isoprene according to methods described in US 2012/0045807. In one example, the microorganisms can be used for the production of isoprene by microbial fermentation of a substrate comprising CO according to methods described in US 2013/0323820.

Once produced, any method can be used to isolate isoprene. For example, isoprene can be recovered from the fermenter off-gas stream as a volatile product as the boiling point of isoprene is 34.1° C. At a typical fermentation temperature of approximately 30° C., isoprene has a high vapor pressure and can be stripped by the gas flow rate through the broth for recovery from the off-gas. Isoprene can be selectively adsorbed onto, for example, an adsorbent and separated from the other off-gas components. Membrane separation technology may also be employed to separate isoprene from the other off-gas compounds. Isoprene may desorbed from the adsorbent using, for example, nitrogen and condensed at low temperature and high pressure.

The invention is further described in the following examples, which do not limit the scope of the invention described in the claims.

### EXAMPLES

The mevalonate pathway for the conversion of acetyl-CoA to the isoprenoid precursors, isopentenyl diphosphate (IPP) and dimethylallyl diphosphate (DMAPP), is found in eukaryotes, archaea, and some bacteria, but is absent from the facultative chemolithotrophic bacterium, Cupriavidus necator (previously called Hydrogenomonas eutrophus, Alcaligenes eutropha, Ralstonia eutropha, and Wautersia eutropha). To simplify the task of evaluating MVA pathway performance in a heterologous host, the pathway can be tested as two separate modules, the upper MVA pathway, converting acetyl-CoA to (R)-mevalonate, and the lower pathway which converts (R)-mevalonate into DMAPP and IPP.

In the upper mevalonate pathway, two molecules of acetyl-CoA are condensed to form acetoacetyl-CoA by the action of acetoacetyl-CoA C-acetyltransferase. Acetoacetyl-CoA is then converted to HMG-CoA by HMG-CoA synthase and HMG-CoA reductase catalyzes the reduction of HMG-CoA to mevalonate. Mevalonate then feeds in to the lower mevalonate pathway, where it is converted to isopentenyl diphosphate (IPP) and dimethylallyl diphosphate (DMAPP). DMAPP is the immediate precursor of isoprene.

### Example 1: Production of Mevalonate

In E. coli a number of versions of the upper mevalonate pathway have been successfully expressed, including the Enterococcus faecalis and Saccharomyces cerevisiae pathways. The Enterococcus faecalis upper mevalonate pathway to mevalonate was selected for evaluation in C. necator. This exemplification shows the pathway for production of mevalonate is functional in C. necator.

The Enterococcus faecalis upper mevalonate pathway comprises MvaE, which has dual acetoacetyl-CoA C-acetyltransferase and HMG-CoA reductase activity, and MvaS, which has HMG-CoA synthase activity. Synthetic genes encoding these enzymes were codon optimized for expression in C. necator (see polypeptide and nucleotide sequences in FIGS. 4 and 5).

For expression of the upper mevalonate pathway the plasmid pBBR1-1A-Pbad-441-442-443 was constructed (FIG. 2). This plasmid contains a synthetic operon comprising the synthetic mvaE and mvaS genes (together with ribosome binding sites) under the control of the E. coli araBAD promoter. The plasmid has the pBBR1 replicon and has a kanamycin resistance gene for selection in E. coli and C. necator.

Plasmids pBBR1-1A-Pbad-441-442-443 (FIG. 2) and an empty vector control plasmid, pBBR1-1A, were used to transform Cupriavidus necator H16 ΔphaCAB to kanamycin resistance. Strains Cupriavidus necator H16 ΔphaCAB::pBBR1-1A-Pbad-441-442-443 and Cupriavidus necator H16 ΔphaCAB::pBBR1-1A were grown in 5 mL Tryptone Soy Broth without Dextrose (Sigma T3938: 17 g/L casein enzymatic hydrolysate; 3 g/L papaic digest of soybean meal; 5 g/L sodium chloride; 2.5 g/L dipotassium phosphate) at 30° C., 220 rpm for 16 hours. 100 μL of these cultures were used to inoculate 5 mL of Cupriavidus defined medium (1.15 g/L KH₂PO₄; 1.15 g/L Na₂HPO₄; 1 g/L NH₄Cl; 0.5 g/L MgSO₄.7H₂O; 0.062 g/L CaCl₂.2H₂O; 5 g/L fructose; 15 mg/L FeSO₄.7H₂O; 2.4 mg/L MnSO₄.H₂O; 2.4 mg/L ZnSO₄.7H₂O; 0.48 mg/L CuSO₄.5H₂O) with and without the addition of 1 g/L L-arabinose to induce expression from the araBAD promoter. These cultures wore incubated at 30° C., 220 rpm for 48 hours.

Culture broths were clarified by centrifugation 10,000×G for 10 minutes. Culture broth (0.5 mL) was acidified with 0.2 mL 0.5M HCl and agitated at 1400 rpm for 15 minutes to convert all the mevalonate to mevalonolactone. The mevalonolactone was extracted from the aqueous phase by the addition of 0.5 mL of ethyl acetate and the samples were agitated at 1400 rpm for a further 15 minutes. The ethyl acetate used for the extraction contained an internal standard, caryophyllene, at a concentration 10 ppM, for data normalization. The use of caryophyllene as an internal standard has previously been reported see Douglas J. Pitera, Chris J. Paddon, Jack D. Newman, Jay D. Keasling, Balancing a heterologous mevalonate pathway for improved isoprenoid production in Escherichia coli, Metabolic Engineering 9 (2007) 193-207. All samples, including the standards were treated in the same way. Following extraction, 1 μl of the top layer was injected onto an Agilent (Santa Clara, Calif.) 7890B GC coupled town Agilent quadrupole 5977A MSD instrument with an electronically controlled split/split-less injection port. The instrument was equipped with a Gerstel (Mülheim, Germany) dual head MPS autosampler for head space analysis. The GCMS parameters used to measure mevalonolactone are presented in table 1.

TABLE 1
GCMS parameters used to measure mevalonolactone
(upper MVA pathway)

| PARAMETER | VALUE | |
|---|---|---|
| Carrier Gas | Helium at constant flow (1.0 ml/min) | |
| Injector | Split ratio | Split less |
| | Temperature | 230° C. |
| Detector | Source Temperature | 230° C. |
| | Quad Temperature | 150° C. |
| | Interface | 260° C. |
| | Gain | 1 |
| | Scan Range | m/z 27-300 |
| | Threshold | 150 |
| | Scan Speed 2\hat{ }2(A/D | 4 |
| | samples) | |
| | Sampling Rate 2\hat{ }n = 2\hat{ }2 | |
| | Mode | SCAN and SIM |

| | |
|---|---|
| Solvent delay * | 5.0 min |
| Oven Temperature | Initial T: 90° C. × 2 min |
| Oven Ramp | 40° C./min to 260° C. for |
| | 12 min |
| Injection volume | 1 μl from the the top organic layer in the 2 ml GC |
| | vial |
| Gas saver | On after 2 min |
| Concentration range | 0.601-76.96 |
| (μg/ml) | |
| GC Column | DB-624 122-1334 Agilent) 30 m × 250 μm × |
| | 1.4 μm |

The method used for the analysis converted all mevalonate to the lactone prior to GC. Therefore, mevalonolactone (rather than mevalonate) was detected.

The presence of mevalonolactone in samples was confirmed by comparison of retention time and ion ratios to those of authentic standards. Authentic samples of mevalonate were used to prepare standard curves for quantification of samples using SIM (selected ion—43 m/z). All data from standards and samples were normalized to the internal standard caryophyllene (selected ion—93 m/z).

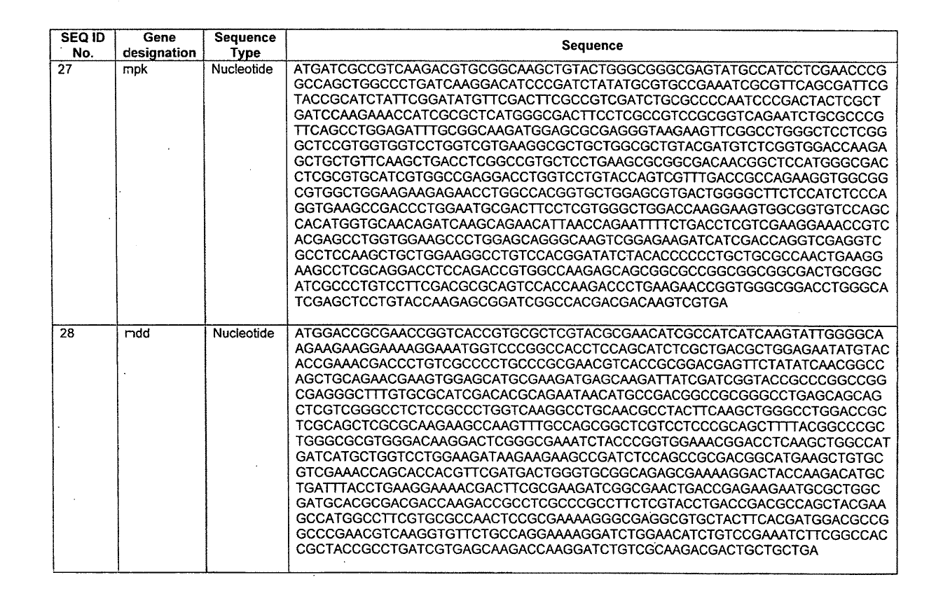

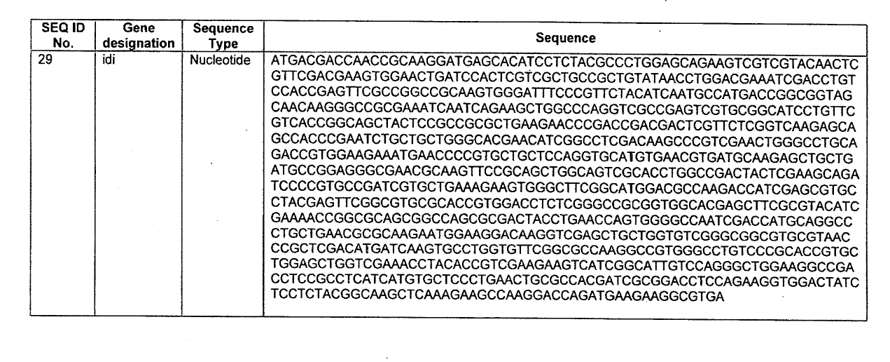

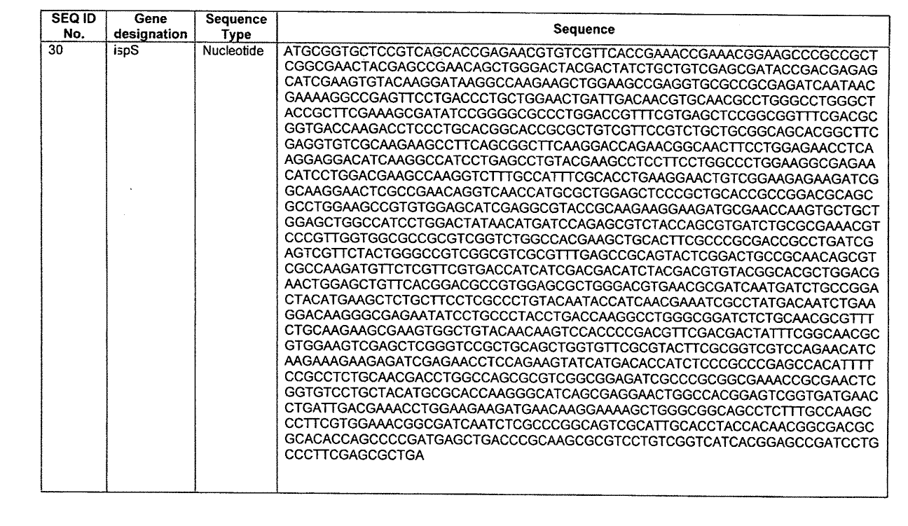

Following growth for 48 hours, mevalonolactone levels were measured (see FIG. 6). When expression of MvaE and MvaS was induced with L-arabinose, mevalonate was detected at 373 ppm. In the absence of induction with L-arabinose, no mevalonolactone was detected. In the strains transformed with the empty plasmid, no mevalonate was detected irrespective of the presence of L-arabinose.

These results indicated that the Enterococcus faecalis upper MVA pathway was able to produce mevalonate in live C. necator cells, thus confirming the functionality of the upper MVA pathway in C. necator.

### Example 2: Production of Isoprene

The Streptococcus pneumoniae lower mevalonate pathway converting mevalonate to the isoprene precursor dimethylallyl diphosphate (DMAPP), was selected for evaluation in C. necator. The pathway consists of mevalonate kinase (MK, EC 2.7.1.36), phosphomevalonate kinase (MPK, EC 2.7.1.36), mevalonate diphosphate decarboxylase (MDD, EC 4.1.1.33), and isopentenyl diphosphate isomerase (IPP, EC 5.3.3.2). The performance of the S. pneumoniae lower mevalonate pathway was monitored in live C. necator cells by converting DMAPP to isoprene with a truncated version of the Populus alba isoprene synthase (IspS, EC 4.2.3.27) containing a deletion of the amino-terminal chloroplast targeting sequence (residues 1 to 36). Synthetic genes encoding these enzymes were codon optimized for expression in C. necator (see polypeptide and nucleotide sequences in FIGS. 4 and 5).

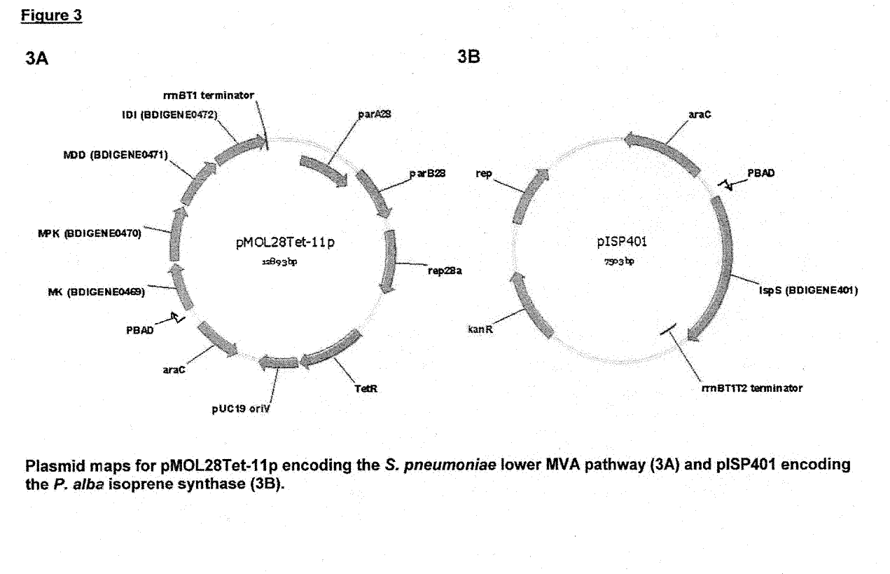

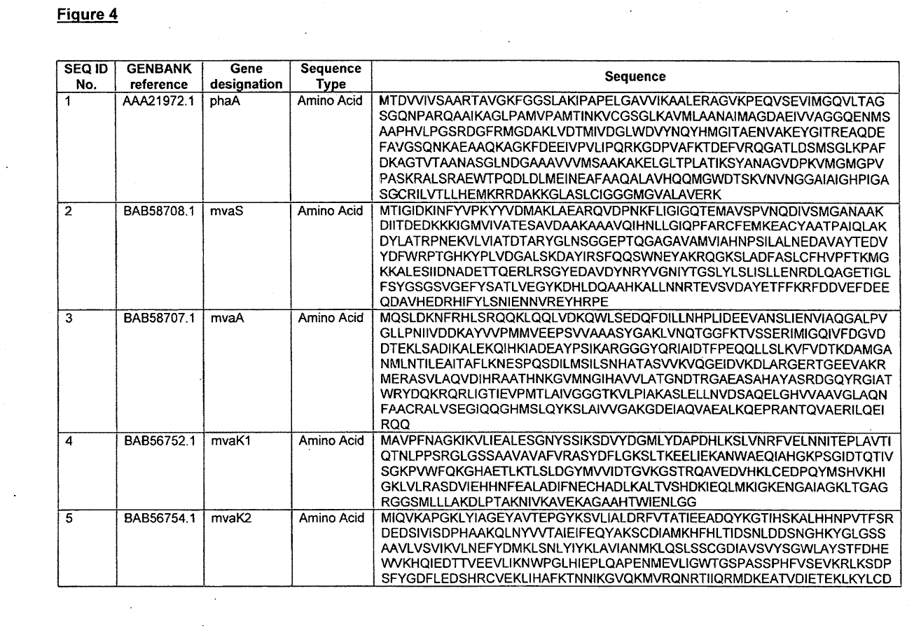

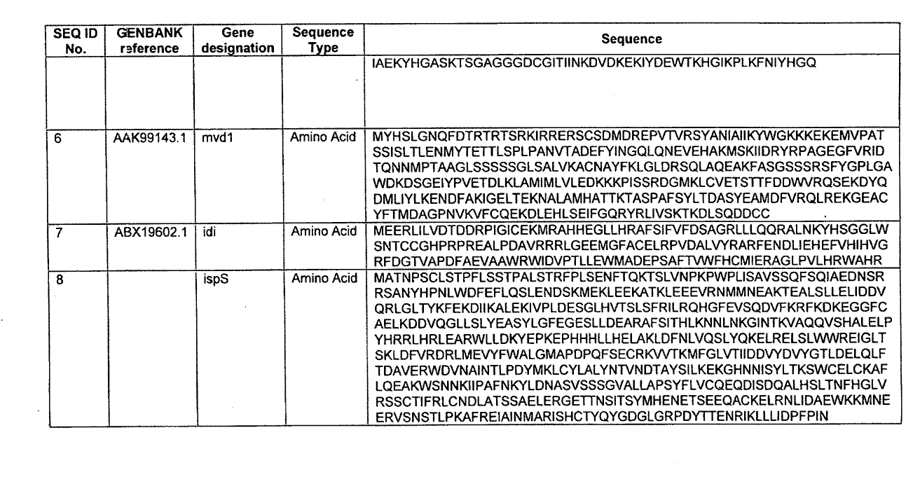

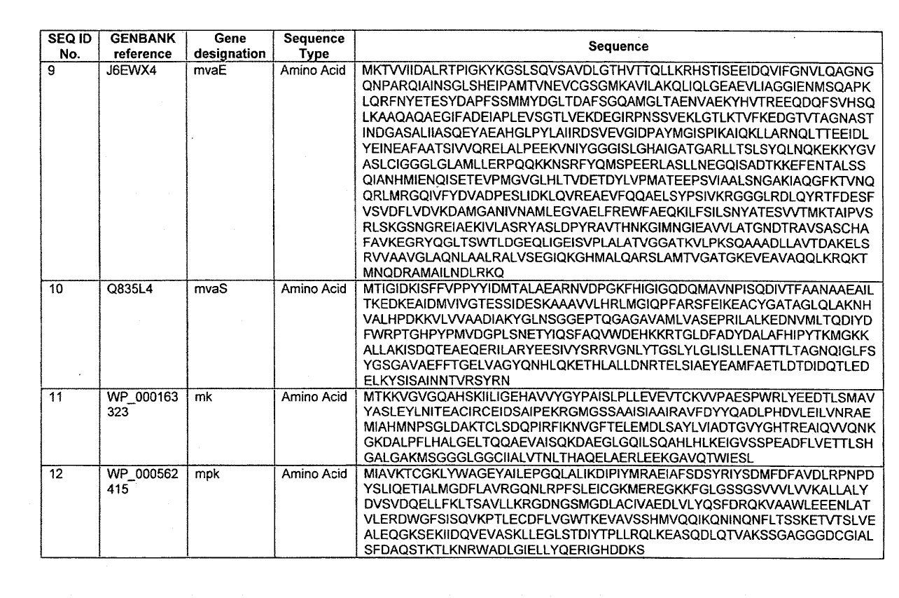

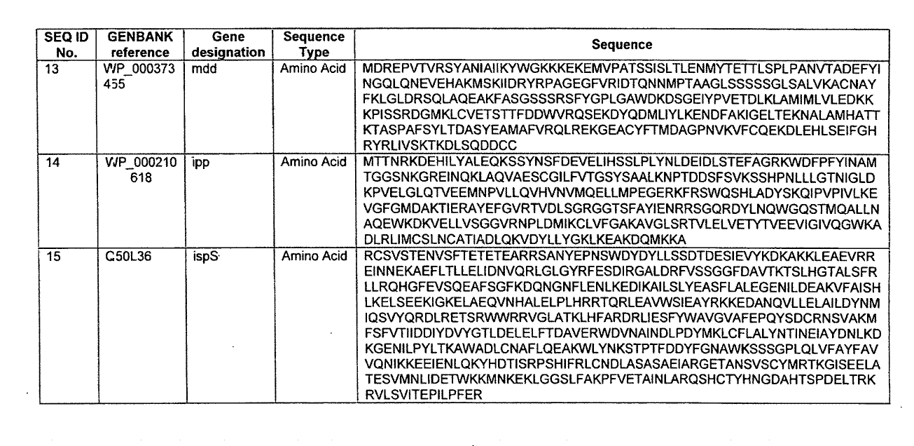

To test the functionality of the lower MVA pathway in C. necator, a synthetic operon was constructed encoding the S. pneumoniae lower MVA pathway enzymes (EC 2.7.1.36, EC 2.7.4.2, EC 4.1.1.33, and EC 5.3.3.2), under the control of the arabinose-inducible P_{BAD} promoter. The resulting pMOL28Tet-11p plasmid (see FIG. 3A), conferred tetracycline resistance and contained two origins of replication, from plasmids pUC19 and Cupriavidus metallidurans pMOL28, for replication in E. coli and C. necator, respectively. The synthetic P. alba IspS gene, expressing residues 37 to 595 of the wild type Populus alba isoprene synthase, was expressed from a second araBAD promoter on a pBBR122-based plasmid, pISP401 (FIG. 3B), conferring kanamycin resistance.

Plasmids pMOL28Tet-11p and pISP401 were co-transformed into a AphaCAB mutant of C. necator H16. A no-isoprene synthase control strain was also constructed by co-transforming pMOL28Tet-11p with a pBBR122-based plasmid, pBBR1 1A-pTac-crtE-crtB-crtl-rrnBt1T2.

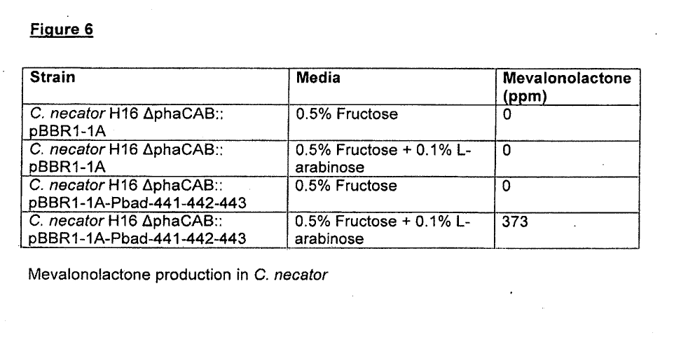

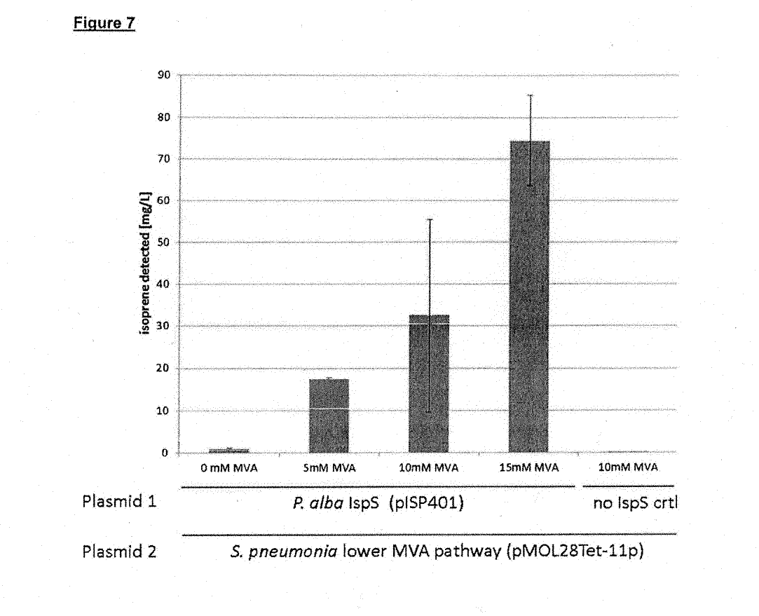

Strains C. necator H16 ΔphaCAB::(pMOL28Tet-11p+pISP401) and C. necator H16 ΔphaCAB::(pMOL28Tet-11p+pBBR1 1A-pTac-crtE-crtB-crtl-rrnBt1T2) were evaluated for isoprene production in a whole-cell mevalonate bioconversion assay (see FIG. 7).

Seeding cultures of the two strains were prepared by inoculating a single colony into 20 ml of 27.5 g/L Tryptone Soya broth without Dextrose (TSB-D media, Sigma Aldrich catalogue number T3938-500G) containing the appropriate antibiotics. The seeding cultures were incubated at 30° C., 230 rpm for 48 hours, then diluted by 1 in 50 into fresh TSB-D media (50 ml) in a 250 ml flask and incubated for approximately 6 hours at 30° C., 230 rpm. The lower MVA pathway and isoprene synthase expression were induced by adding arabinose to a final concentration of 1% w/v and the cultures were incubated for a further 16 hours (overnight) at 30° C., 230 rpm. The cultures were pelleted by centrifugation at 6000 g for 20 minutes and wet cell weight was measured for each cell pellet. The density of each culture was normalized to 0.2 g WCW/ml by re-suspending the C. necator cells with the appropriate volume of TSB-D medium containing 1% arabinose and antibiotics.

Mevalonate bioconversion assays were set up in triplicate for each strain and mevalonate concentration tested, using 10 ml screw cap GC-MS vials. Each GC-MS vial contained 2 ml fresh TSB-D media (with 1% w/v arabinose and appropriate antibiotics), 20 μl of 0.2 g WCW/ml of either C. necator H16 ΔphaCAB::(pMOL28Tet-11p+pISP401) or C. necator H16 ΔphaCAB::(pMOL28Tet-11p+pBBR1 1A-pTac-crtE-crtB-crtl-rrnBt1T2) and R-Mevalonic acid lithium salt (Sigma 50838-50MG) added to final concentrations ranging from 0 mM to 15 mM. An isoprene calibration series was set up in 10 ml GC-MS vials containing 1990 μl TSB-D media with 10 μl of 20 ppm to 1000 ppm of isoprene standards dissolved in 0.5% v/v methanol at 4° C. To test assay robustness and precision, spike-recovery vials were also set up containing 10 μl of 1 ppm isoprene, for a random selection of 7 of the experimental conditions tested. All vials (experimental, isoprene standard and spike recovery vials) were incubated at 30° C., 160 rpm, for 24 hours and isoprene was measured in the headspace by gas chromatography mass spectroscopy (GC-MS).

Headspace isoprene measurements were performed by GC-MS on an Agilent Technologies 7890B gas chromatograph connected to an Agilent quadrupole 5977A MSD instrument with an electronically controlled split/split-less injection port. The instrument was equipped with a dual head MPS autosampler (Gerstel) for head space analysis. GC separation was performed on a db-624 capillary column (60 m×0.25 mm×1.4 μm J&W Scientific). The GC-MS parameters were as described in Table 2. The M-1 ion was used for isoprene quantification.

TABLE 2
GCMS parameters used to measure head space
isoprene concentrations (lower MVA pathway)

| PARAMETER | VALUE | |
|---|---|---|
| Carrier Gas | Helium at constant flow (2.0 ml/min) | |
| Injector | Split ratio | Split 10:L |
| | Temperature | 150° C. |
| Detector | Source Temperature | 230° C. |
| | Quad Temperature | 150° C. |
| | Interface | 260° C. |
| | Gain | 1 |
| | Scan Range | m/z 28-200 |
| | Threshold | 150 |
| | Scan Speed 2\hat{ }2(A/D | 4 |
| | samples) | |
| | Sampling Rate | |
| | 2\hat{ }n = 2\hat{ }2 | |
| | Mode | SCAN and SIM |

| | |
|---|---|
| Solvent delay * | 5.50 min |
| Oven Temperature | Initial T: 40° C. × 10 min |
| Oven Ramp | 40° C./min to 260° C. for 5 min |
| Injection volume | 500 μl from the HS in the GC 2 ml vial |
| Incubation time and T | 15 min at 95° C. |
| Agitator | ON 500 rpm |
| Injection volume | 500 μl of the Head Space |
| Gas saver | On after 2 min |
| Concentration range | 0.1-5.0 |
| (μg/ml) | |
| GC Column | DB-624 122-1334 Agilent) 60 m × 250 μm × |
| | 1.4 μm |

FIG. 7 shows in vivo bioconversion of (R)-mevalonate to isoprene in a C. necator H16 ΔphaCAB strain expressing the S. pneumonia lower MVA pathway and P. alba isoprene synthase. Error bars represent standard deviation (n=3). Full name of ‘no IspS’ control plasmid is pBBR1 1A-pTac-crtE-crtB-crtl-rrnBT1T2. The strain was fed increasing concentrations of mevalonate (0 mM to 15 mM R-Mevalonic acid) in 10 ml GC-MS vials, resulting in average isoprene titers of 17 mg/L, 33 mg/L and 74 mg/L from 5 mM, 10 mM and 15 mM Mevalonate, respectively (FIG. 7). By contrast, less than 1 mg/L isoprene was detected in the culture vials without mevalonate supplementation. Isoprene was undetectable from a no-isoprene synthase control strain fed with 10 mM mevalonate, confirming that the GC-MS assay was monitoring isoprene in the head space. These results indicated that the S. pneumoniae lower MVA pathway, with an isoprene synthase, was able to bio-convert mevalonate to isoprene in live C. necator cells, thus confirming the functionality of the lower MVA pathway in C. necator.

### Example 3: Production of Isoprene

The amino acid sequence of the acetyl-CoA acetyltransferase derived from Cupriavidus necator is known (Genbank Accession No. AAA21972.1, amino acid sequence SEQ ID No: 1).

The amino acid sequence of the hydroxymethylglutaryl-CoA synthase derived from Staphylococcus aureus is known (Genbank Accession No. BAB58708.1, amino acid sequence SEQ ID No: 2).

The amino acid sequence of the hydroxymethylglutaryl Co-A reductase derived from Staphylococcus aureus is known (Genbank Accession No. BAB58707.1, amino acid sequence SEQ ID No: 3).

The amino acid sequence of the mevalonate-kinase derived from Staphylococcus aureus is known (Genbank Accession No. BAB56752.1, amino acid sequence SEQ ID No: 4).

The amino acid sequence of the phosphomevalonate kinase derived from Staphylococcus aureus is known (Genbank Accession No. BAB56754.1, amino acid sequence SEQ ID No: 5).

The amino acid sequence of the diphosphomevalonate decarboxylase derived from Streptococcus pneumoniae is known (Genbank Accession No. AAK99143.1, amino acid sequence SEQ ID No: 6).

The amino acid sequence of the isopentyl diphosphate isomerase derived from B. multivorans is known (Genbank Accession No. ABX19602.1, amino acid sequence SEQ ID No: 7).

The amino acid sequence of the isoprene synthase derived from Mucuna pruriens is known (amino acid sequence SEQ ID No: 8).

The amino acid sequence of the enzyme having dual acetoacetyl-CoA C-acetyltransferase and HMG-CoA reductase activity from Enterococcus faecalis is known (Genbank Accession No. J6EWX4, SEQ ID No: 9).

The amino acid sequence of the hydroxymethylglutaryl-CoA synthase derived from Enterococcus faecalis is known (Genbank Accession No. Q835L4, SEQ ID No: 10).

The amino acid sequence of the mevalonate-kinase derived from Streptococcus pneumoniae is known (Accession No. WP_000163323, SEQ ID No: 11).

The amino acid sequence of the phosphomevalonate kinase derived from Streptococcus pneumoniae is known (Accession No. WP_000562415, SEQ ID No: 12).

The amino acid sequence of the diphosphomevalonate decarboxylase derived from Streptococcus pneumoniae is known (Accession No. WP_000373455, SEQ ID No: 13).

The amino acid sequence of the isopentyl diphosphate isomerase derived from Streptococcus pneumoniae is known (Accession No. WP_000210618, SEQ ID No: 14).

The amino acid sequence of the isoprene synthase derived from Populus alba is known (Accession No. Q50L36, SEQ ID No: 15).

Each of those genes is obtained by PCR amplification using genomic DNA (gDNA) templates and custom oligonucleotide primers. All gDNAs are prepared using standard genomic DNA purification techniques. Recombinant DNA techniques to insert the amplified genes into suitable expression vectors are performed according to standard procedures and using standard restriction enzymes. Compatible vectors are used to provide individual expression of each gene in a Cupriavidus necator host. The PCR products are digested with restriction enzymes corresponding to the restriction site incorporated into them by their respective primers and ligated directly into similarly digested vectors using standard ligation techniques. All constructs are confirmed to be correct by restriction enzyme digestion and/or nucleotide sequencing. Once the plasmids are constructed, some or all are used to co-transform a C. necator host according to standard procedures to create a production strain. The transformed host is cultured in a CO₂/H₂ gas medium with suitable parameters in a continuous process, and isoprene is recovered via a harvesting step.

### ADDITIONAL EXEMPLARY EMBODIMENTS

In one embodiment is provided a method for synthesizing unsaturated pentahydrocarbons, for example isoprene and intermediates thereof.

In one embodiment, the method for synthesizing isoprene comprises: enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or SEQ ID No: 10 or a functional fragment of said enzyme; and enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or SEQ ID No: 9 a functional fragment of said enzyme. In one embodiment, the method further comprises at least one of: enzymatically converting acetyl-CoA to acetoacetyl-CoA using a polypeptide having the activity of an acetyl-CoA acetyltransferase enzyme having the amino acid sequence set forth in SEQ ID No: 1 or SEQ ID No: 9 or a functional fragment of said enzyme; enzymatically converting (R)-mevalonate to (R)-5-phosphomevalonate using a polypeptide having the activity of a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using a polypeptide having the activity of an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having the amino acid sequence set forth in SEQ ID No: 8 or SEQ ID No: 15 or a functional fragment of said enzyme. In one embodiment, the method further comprises: enzymatically converting acetyl-CoA to acetoacetyl-CoA using a polypeptide having the activity of an acetyl-CoA acetyltransferase enzyme having the amino acid sequence set forth in SEQ ID No: 1 or SEQ ID No: 9 or a functional fragment of said enzyme; enzymatically converting (R)-mevalonate to (R)-5-phosphomevalonate using a polypeptide having the activity of a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using a polypeptide having the activity of an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having the amino acid sequence set forth in SEQ ID No: 8 or SEQ ID No: 15 or a functional fragment of said enzyme.

In one embodiment, the method for synthesizing isoprene comprises: enzymatically converting acetoacetyl-CoA to 3-hydroxy-3-methylglutaryl-CoA using a hydroxymethylglutaryl-CoA synthase enzyme having the amino acid sequence set forth in SEQ ID No: 2 or SEQ ID No: 10 or a functional fragment of said enzyme; and enzymatically converting 3-hydroxy-3-methylglutaryl-CoA to (R)-mevalonate using a hydroxymethylglutaryl Co-A reductase enzyme having the amino acid sequence set forth in SEQ ID No: 3 or SEQ ID No: 9 a functional fragment of said enzyme. In one embodiment, the method further comprises at least one of: enzymatically converting acetyl-CoA to acetoacetyl-CoA using an acetyl-CoA acetyltransferase enzyme having the amino acid sequence set forth in SEQ ID No: 1 or SEQ ID No: 9 or a functional fragment of said enzyme; enzymatically converting (R)-mevalonate to (R)-5-phosphomevalonate using a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using an isoprene synthase enzyme having the amino acid sequence set forth in SEQ ID No: 8 or SEQ ID No: 15 or a functional fragment of said enzyme. In one embodiment, the method further comprises: enzymatically converting acetyl-CoA to acetoacetyl-CoA using an acetyl-CoA acetyltransferase enzyme having the amino acid sequence set forth in SEQ ID No: 1 or SEQ ID No: 9 or a functional fragment of said enzyme; enzymatically converting (R)-mevalonate to (R)-5-phosphomevalonate using a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using an isoprene synthase enzyme having the amino acid sequence set forth in SEQ ID No: 8 or SEQ ID No: 15 or a functional fragment of said enzyme.

In one embodiment, the method for synthesizing isoprene comprises: enzymatically converting acetoacetyl-CoA to 3-hydroxy-3-methylglutaryl-CoA using a polypeptide having the activity of a hydroxymethylglutaryl-CoA synthase enzyme having having at least 90%, 91%, 92%, 93%, 94%, 95%, 96%, 97%, 98%, or 99% sequence identity to the amino acid sequence set forth in SEQ ID No: 2 or SEQ ID No: 10 or a functional fragment of said enzyme; and enzymatically converting 3-hydroxy-3-methylglutaryl-CoA to (R)-mevalonate using a polypeptide having the activity of a hydroxymethylglutaryl Co-A reductase enzyme having having at least 90%, 91%, 92%, 93%, 94%, 95%, 96%, 97%, 98%, or 99% A sequence identity to the amino acid sequence set forth in SEQ ID No: 3 or SEQ ID No: 9 or a functional fragment of said enzyme. In one embodiment, the method further comprises at least one of: enzymatically converting acetyl-CoA to acetoacetyl-CoA using a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98%, or 99% A sequence identity to the amino acid sequence set forth in SEQ ID No: 4 or a functional fragment of said enzyme; enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme. In one embodiment, the method further comprises: enzymatically converting acetyl-CoA to acetoacetyl-CoA using a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; enzymatically converting (R)-mevalonate to (R)-5-phosphomevalonate a polypeptide having the activity of using a mevalonate-kinase enzyme having at least 90%, 91%, 92%, 93%, 94%, 95%, 96%, 97%, 98%, or 99% sequence identity to the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme.

In one embodiment, the method for synthesizing isoprene comprises: enzymatically converting acetoacetyl-CoA to 3-hydroxy-3-methylglutaryl-CoA using a hydroxymethylglutaryl-CoA synthase enzyme having having at least 90%, 91%, 92%, 93%, 94%, 95%, 96%, 97%, 98%, or 99% sequence identity to the amino acid sequence set forth in SEQ ID No: 2 or SEQ ID No: 10 or a functional fragment of said enzyme; and enzymatically converting 3-hydroxy-3-methylglutaryl-CoA to (R)-mevalonate using a hydroxymethylglutaryl Co-A reductase enzyme having having at least 90%, 91%, 92%, 93%, 94%, 95%, 96%, 97%, 98%, or 99% sequence identity to the amino acid sequence set forth in SEQ ID No: 3 or SEQ ID No: 9 or a functional fragment of said enzyme. In one embodiment, the method further comprises at least one of: enzymatically converting acetyl-CoA to acetoacetyl-CoA using an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; enzymatically converting (R)-mevalonate to (R)-5-phosphomevalonate using a mevalonate-kinase enzyme having at least 90%, 91%, 92%, 93%, 94%, 95%, 96%, 97%, 98%, or 99% sequence identity to the amino acid sequence set forth in SEQ ID No: 4 or a functional fragment of said enzyme; enzymatically converting (R)-5-phosphomevalonate to (R)-5-diphosphomevalonate using a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and enzymatically converting dimethylallyl diphosphate to isoprene using an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme. In one embodiment, the method further comprises: enzymatically converting acetyl-CoA to acetoacetyl-CoA using an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; enzymatically converting (R)-mevalonate to (R)-5-phosphomevalonate using a mevalonate-kinase enzyme having at least 90%, 91%, 92%, 93%, 94%, 95%, 96%, 97%, 98%, or 99% sequence identity to the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and enzymatically converting dimethylallyl diphosphate to isoprene using an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme.

In one embodiment, the non-naturally occurring chemolithotrophic host is capable of producing isoprene via the mevalonate pathway and comprises: at least one exogenous nucleic acid encoding a polypeptide having the activity of a hydroxymethylglutaryl-CoA synthase enzyme having the amino acid sequence set forth in SEQ ID No: 2 or SEQ ID No: 10 or a functional fragment of said enzyme; and at least one exogenous nucleic acid encoding a polypeptide having the activity of a hydroxymethylglutaryl Co-A reductase enzyme having the amino acid sequence set forth in SEQ ID No: 3 or SEQ ID No: 9 or a functional fragment of said enzyme. In one embodiment the host further comprises at least one of: at least one exogenous nucleic acid encoding a polypeptide having the activity of an acetyl-CoA acetyltransferase enzyme having the amino acid sequence set forth in SEQ ID No: 1 or SEQ ID No: 9 or a functional fragment of said enzyme; at least one exogenous nucleic acid encoding a polypeptide having the activity of a mevalonate-kinase enzyme having the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having the amino acid sequence set forth in SEQ ID No: 7 or SEQ ID No: 14 or a functional fragment of said enzyme; and at least one exogenous nucleic acid encoding a polypeptide having the activity of an isoprene synthase enzyme having the amino acid sequence set forth in SEQ ID No: 8 or SEQ ID No: 15 or a functional fragment of said enzyme. In one embodiment the host further comprises: at least one exogenous nucleic acid encoding a polypeptide having the activity of an acetyl-CoA acetyltransferase enzyme having the amino acid sequence set forth in SEQ ID No: 1 or SEQ ID No: 9 or a functional fragment of said enzyme; at least one exogenous nucleic acid encoding a polypeptide having the activity of a mevalonate-kinase enzyme having the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having the amino acid sequence set forth in SEQ ID No: 7 or SEQ ID No: 14 or a functional fragment of said enzyme; and at least one exogenous nucleic acid encoding a polypeptide having the activity of an isoprene synthase enzyme having the amino acid sequence set forth in SEQ ID No: 8 or SEQ ID No: 15 or a functional fragment of said enzyme.

In one embodiment, the non-naturally occurring chemolithotrophic host is capable of producing isoprene via the mevalonate pathway and comprises: at least one exogenous nucleic acid encoding a hydroxymethylglutaryl-CoA synthase enzyme having the amino acid sequence set forth in SEQ ID No: 2 or SEQ ID No: 10 or a functional fragment of said enzyme; and at least one exogenous nucleic acid encoding a hydroxymethylglutaryl Co-A reductase enzyme having the amino acid sequence set forth in SEQ ID No: 3 or SEQ ID No: 9 or a functional fragment of said enzyme. In one embodiment the host further comprises at least one of: at least one exogenous nucleic acid encoding an acetyl-CoA acetyltransferase enzyme having the amino acid sequence set forth in SEQ ID No: 1 or SEQ ID No: 9 or a functional fragment of said enzyme; at least one exogenous nucleic acid encoding a mevalonate-kinase enzyme having the amino acid sequence set forth in SEQ ID No: 4 or SEQ ID No: 11 or a functional fragment of said enzyme; at least one exogenous nucleic acid encoding a phosphomevalonate kinase enzyme having the amino acid sequence set forth in SEQ ID No: 5 or SEQ ID No: 12 or a functional fragment of said enzyme; at least one exogenous nucleic acid encoding a diphosphomevalonate decarboxylase enzyme having the amino acid sequence set forth in SEQ ID No: 6 or SEQ ID No: 13 or a functional fragment of said enzyme; at least one exogenous nucleic acid encoding an isopentenyl diphosphate isomerase enzyme having the amino acid sequence set forth in SEQ ID No: 7 or SEQ ID No: 14 or a functional fragment of said enzyme; and at least one exogenous nucleic acid encoding an isoprene synthase enzyme having the amino acid sequence set forth in SEQ ID No: 8 or SEQ ID No: 15 or a functional fragment of said enzyme. In one embodiment the host further comprises: at least one exogenous nucleic acid encoding an acetyl-CoA acetyltransferase enzyme having the amino acid sequence set forth in SEQ ID No: 1 or SEQ ID No: 9 or a functional fragment of said enzyme; at least one exogenous nucleic acid encoding a mevalonate-kinase enzyme having the amino acid sequence set forth in SEQ ID No: 4 or SEQ ID No: 11 or a functional fragment of said enzyme; at least one exogenous nucleic acid encoding a phosphomevalonate kinase enzyme having the amino acid sequence set forth in SEQ ID No: 5 or SEQ ID No: 12 or a functional fragment of said enzyme; at least one exogenous nucleic acid encoding a diphosphomevalonate decarboxylase enzyme having the amino acid sequence set forth in SEQ ID No: 6 or SEQ ID No: 13 or a functional fragment of said enzyme; at least one exogenous nucleic acid encoding an isopentenyl diphosphate isomerase enzyme having the amino acid sequence set forth in SEQ ID No: 7 or SEQ ID No: 14 or a functional fragment of said enzyme; and at least one exogenous nucleic acid encoding an isoprene synthase enzyme having the amino acid sequence set forth in SEQ ID No: 8 or SEQ ID No: 15 or a functional fragment of said enzyme.

In one embodiment, the non-naturally occurring chemolithotrophic host is capable of producing isoprene via the mevalonate pathway and comprises: at least one exogenous nucleic acid encoding a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 2 or SEQ ID No: 10 or a functional fragment of said enzyme; and at least one exogenous nucleic acid encoding a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 3 or SEQ ID No: 9 or a functional fragment of said enzyme. In one embodiment the host further comprises at least one of: at least one exogenous nucleic acid encoding a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme. In one embodiment the host further comprises: at least one exogenous nucleic acid encoding a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme.

In one embodiment, the non-naturally occurring chemolithotrophic host is capable of producing isoprene via the mevalonate pathway and comprises: at least one exogenous nucleic acid encoding a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 2 or SEQ ID No: 10 or a functional fragment of said enzyme; and at least one exogenous nucleic acid encoding a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 3 or SEQ ID No: 9 or a functional fragment of said enzyme. In one embodiment the host further comprises at least one of: at least one exogenous nucleic acid encoding an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; at least one exogenous nucleic acid encoding a mevalonate-kinase enzyme having at least 90%, 91%, 92%, 93%, 94%, 95%, 96%, 97%, 98%, or 99% sequence identity to the amino acid sequence set forth in SEQ ID No: 4 or SEQ ID No: 11 or a functional fragment of said enzyme; at least one exogenous nucleic acid encoding a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; at least one exogenous nucleic acid encoding a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; at least one exogenous nucleic acid encoding an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and at least one exogenous nucleic acid encoding an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme. In one embodiment the host further comprises: at least one exogenous nucleic acid encoding an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or SEQ ID No: 9 or a functional fragment of said enzyme; at least one exogenous nucleic acid encoding a mevalonate-kinase enzyme having at least 90%, 91%, 92%, 93%, 94%, 95%, 96%, 97%, 98%, or 99% sequence identity to the amino acid sequence set forth in SEQ ID No: 4 or SEQ ID No: 11 or a functional fragment of said enzyme; at least one exogenous nucleic acid encoding a phosphomevalonate kinase enzyme having at least 90%, 91%, 92%, 93%, 94%, 95%, 96%, 97%, 98%, or 99% sequence identity to the amino acid sequence set forth in SEQ ID No: 5 or SEQ ID No: 12 or a functional fragment of said enzyme; at least one exogenous nucleic acid encoding a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or SEQ ID No: 13 or a functional fragment of said enzyme; at least one exogenous nucleic acid encoding an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme; and at least one exogenous nucleic acid encoding an isoprene synthase enzyme having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme.

In one embodiment, the method for synthesizing isoprene comprises: enzymatically converting acetyl-CoA to acetoacetyl-CoA using a polypeptide having the activity of an acetyl-CoA acetyltransferase enzyme having the amino acid sequence set forth in SEQ ID No: 1 or a SEQ ID No: 9 or functional fragment of said enzyme; enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or SEQ ID No: 9 or a functional fragment of said enzyme; enzymatically converting (R)-mevalonate to (R)-5-phosphomevalonate using a polypeptide having the activity of a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using a polypeptide having the activity of an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having the amino acid sequence set forth in SEQ ID No: 8 or SEQ ID No: 15 or a functional fragment of said enzyme.

In one embodiment, the method for synthesizing isoprene comprises: enzymatically converting acetyl-CoA to acetoacetyl-CoA using an acetyl-CoA acetyltransferase enzyme having the amino acid sequence set forth in SEQ ID No: 1 or a SEQ ID No: 9 or functional fragment of said enzyme; enzymatically converting acetoacetyl-CoA to 3-hydroxy-3-methylglutaryl-CoA using a hydroxymethylglutaryl-CoA synthase enzyme having the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a hydroxymethylglutaryl Co-A reductase enzyme having the amino acid sequence set forth in SEQ ID No: 3 or SEQ ID No: 9 or a functional fragment of said enzyme; enzymatically converting (R)-mevalonate to (R)-5-phosphomevalonate using a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using an isoprene synthase enzyme having the amino acid sequence set forth in SEQ ID No: 8 or SEQ ID No: 15 or a functional fragment of said enzyme.

In one embodiment, the method for synthesizing isoprene comprises: enzymatically converting acetyl-CoA to acetoacetyl-CoA using a polypeptide having the activity of an acetyl-CoA acetyltransferase enzyme having at least 90%, 91%, 92%, 93%, 94%, 95%, 96%, 97%, 98% or 99% sequence identity to the amino acid sequence set forth in SEQ ID No: 1 or SEQ ID No: 9 or a functional fragment of said enzyme; enzymatically converting acetoacetyl-CoA to 3-hydroxy-3-methylglutaryl-CoA using a polypeptide having the activity of a hydroxymethylglutaryl-CoA synthase enzyme having at least 90%, 91%, 92%, 93%, 94%, 95%, 96%, 97%, 98% or 99% sequence identity to the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a polypeptide having the activity of a hydroxymethylglutaryl Co-A reductase enzyme having at least 90%, 91%, 92%, 93%, 94%, 95%, 96%, 97%, 98% or 99% sequence identity to the amino acid sequence set forth in SEQ ID No: 3 or SEQ ID No: 9 or a functional fragment of said enzyme; enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98% or 99% sequence identity to the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98% or 99% sequence identity to the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98% or 99% sequence identity to the amino acid sequence set forth in SEQ ID No: 6 or SEQ ID No: 13 Of a functional fragment of said enzyme; enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having at least 90%, 91%, 92%, 93%, 94%, 95%, 96%, 97%, 98% or 99% sequence identity to the amino acid sequence set forth in SEQ ID No: 8 or SEQ ID No: 15 or a functional fragment of said enzyme.

In one embodiment, the method for synthesizing isoprene comprises: enzymatically converting acetyl-CoA to acetoacetyl-CoA using an acetyl-CoA acetyltransferase enzyme having at least 90%, 91%, 92%, 93%, 94%, 95%, 96%, 97%, 98% or 99% sequence identity to the amino acid sequence set forth in SEQ ID No: 1 or SEQ ID No: 9 or a functional fragment of said enzyme; enzymatically converting acetoacetyl-CoA to 3-hydroxy-3-methylglutaryl-CoA using a hydroxymethylglutaryl-CoA synthase enzyme having at least 90%, 91%, 92%, 93%, 94%, 95%, 96%, 97%, 98% or 99% sequence identity to the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a hydroxymethylglutaryl Co-A reductase enzyme having at least 90%, 91%, 92%, 93%, 94%, 95%, 96%, 97%, 98% or 99% sequence identity to the amino acid sequence set forth in SEQ ID No: 3 or SEQ ID No: 9 or a functional fragment of said enzyme; enzymatically converting (R)-mevalonate to (R)-5-phosphomevalonate using a mevalonate-kinase enzyme having at least 90%, 91%, 92%, 93%, 94%, 95%, 96%, 97%, 98% or 99% sequence identity to the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having at least 90%, 91%, 92%, 93%, 94%, 95%, 96%, 97%, 98% or 99% sequence identity to the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having at least 90%, 91%, 92%, 93%, 94%, 95%, 96%, 97%, 98% or 99% sequence identity to the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and enzymatically converting dimethylallyl diphosphate to isoprene using an isoprene synthase enzyme having at least 90%, 91%, 92%, 93%, 94%, 95%, 96%, 97%, 98% or 99% sequence identity to the amino acid sequence set forth in SEQ ID No: 8 or SEQ ID No: 15 or a functional fragment of said enzyme.

In one embodiment, the method for synthesizing isoprene comprises: enzymatically converting acetyl-CoA to acetoacetyl-CoA using a polypeptide having the activity of an acetyl-CoA acetyltransferase enzyme having the amino acid sequence set forth in SEQ ID No: 1 or SEQ ID No: 9 or a functional fragment of said enzyme; enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or SEQ ID No: 9 or a functional fragment of said enzyme; enzymatically converting (R)-mevalonate to (R)-5-phosphomevalonate using a polypeptide having the activity of a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate a polypeptide having the activity of using an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme classified under EC 4.2.3.27, for example an isoprene synthase having at least 90%, 91%, 92%, 93%, 94%, 95%, 96%, 97%, 98% or 99% sequence identity to the amino acid sequence set forth in SEQ ID No: 8 or SEQ ID No: 15, or a functional fragment of said enzyme.

In one embodiment, the method for synthesizing isoprene comprises: enzymatically converting acetyl-CoA to acetoacetyl-CoA using an acetyl-CoA acetyltransferase enzyme having the amino acid sequence set forth in SEQ ID No: 1 or SEQ ID No: 9 or a functional fragment of said enzyme; enzymatically converting acetoacetyl-CoA to 3-hydroxy-3-methylglutaryl-CoA using a hydroxymethylglutaryl-CoA synthase enzyme having the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a hydroxymethylglutaryl Co-A reductase enzyme having the amino acid sequence set forth in SEQ ID No: 3 or SEQ ID No: 9 or a functional fragment of said enzyme; enzymatically converting (R)-mevalonate to (R)-5-phosphomevalonate using a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having the amino acid sequence set forth in SEQ ID No: 7 or SEQ ID No: 14 or a functional fragment of said enzyme; and enzymatically converting dimethylallyl diphosphate to isoprene using an isoprene synthase enzyme classified under EC 4.2.3.27, for example an isoprene synthase having at least 90%, 91%, 92%, 93%, 94%, 95%, 96%, 97%, 98% or 99% sequence identity to the amino acid sequence set forth in SEQ ID No: 8 or SEQ ID No: 15, or a functional fragment of said enzyme.

In one embodiment, the method for synthesizing isoprene comprises: enzymatically converting acetyl-CoA to acetoacetyl-CoA using a polypeptide having the activity of an acetyl-CoA acetyltransferase enzyme having the amino acid sequence set forth in SEQ ID No: 1 or SEQ ID No: 9 or a functional fragment of said enzyme; enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or SEQ ID No: 9 or a functional fragment of said enzyme; enzymatically converting (R)-mevalonate to (R)-5-phosphomevalonate using a polypeptide having the activity of a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and enzymatically converting dimethylallyl diphosphate to isoprene using a polypeptide having the activity of an isoprene synthase enzyme having the amino acid sequence set forth in SEQ ID No: 8 or SEQ ID No: 15 or a functional fragment of said enzyme. In one embodiment, the method for synthesizing isoprene comprises: enzymatically converting acetyl-CoA to acetoacetyl-CoA using an acetyl-CoA acetyltransferase enzyme having the amino acid sequence set forth in SEQ ID No: 1 or SEQ ID No: 9 or a functional fragment of said enzyme; enzymatically converting acetoacetyl-CoA to 3-hydroxy-3-methylglutaryl-CoA using a hydroxymethylglutaryl-CoA synthase enzyme having the amino acid sequence set forth in SEQ ID No: 2 or SEQ ID No: 10 or a functional fragment of said enzyme; enzymatically converting 3-hydroxy-3-methylglutaryl-CoA to (R)-mevalonate using a hydroxymethylglutaryl Co-A reductase enzyme having the amino acid sequence set forth in SEQ ID No: 3 or SEQ ID No: 9 or a functional fragment of said enzyme; enzymatically converting (R)-mevalonate to (R)-5-phosphomevalonate using a mevalonate-kinase enzyme having the amino acid sequence set forth in SEQ ID No: 4 or SEQ ID No: 11 or a functional fragment of said enzyme; enzymatically converting (R)-5-phosphomevalonate to (R)-5-diphosphomevalonate using a phosphomevalonate kinase enzyme having the amino acid sequence set forth in SEQ ID No: 5 or SEQ ID No: 12 or a functional fragment of said enzyme; enzymatically converting (R)-5-diphosphomevalonate to isopentenyl diphosphate using a diphosphomevalonate decarboxylase enzyme having the amino acid sequence set forth in SEQ ID No: 6 or SEQ ID No: 13 or a functional fragment of said enzyme; enzymatically converting isopentenyl diphosphate to dimethylallyl diphosphate using an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and enzymatically converting dimethylallyl diphosphate to isoprene using an isoprene synthase enzyme having the amino acid sequence set forth in SEQ ID No: 8 or SEQ ID No: 15 or a functional fragment of said enzyme.

In one embodiment, the method for synthesizing isoprene comprises converting isopentenyl diphosphate to dimethylallyl diphosphate using a polypeptide having the activity of an isopentenyl diphosphate isomerase enzyme having an amino acid sequence set forth in SEQ ID No: 7 or SEQ ID No: 14 or a functional fragment of said enzyme.

In one embodiment, the method for synthesizing isoprene comprises converting isopentenyl diphosphate to dimethylallyl diphosphate using an isopentenyl diphosphate isomerase enzyme having an amino acid sequence set forth in SEQ ID No: 7 or SEQ ID No: 14 or a functional fragment of said enzyme.

In one embodiment is provided a non-naturally occurring host capable of producing isoprene via the mevalonate pathway.

In one embodiment, the non-naturally occurring host capable of producing isoprene via the mevalonate pathway comprises at least one exogenous nucleic acid encoding a polypeptide having the activity of a polypeptide having the sequence set forth in SEQ ID No: 1 or SEQ ID No: 9 or a functional fragment of said polypeptide; at least one exogenous nucleic acid encoding a polypeptide having the activity of a polypeptide having the sequence set forth in SEQ ID No: 2 or SEQ ID No: 10 or a functional fragment of said polypeptide; at least one exogenous nucleic acid encoding a polypeptide having the activity of a polypeptide having the sequence set forth in SEQ ID No: 3 or SEQ ID No: 9 or a functional fragment of said polypeptide; at least one exogenous nucleic acid encoding a polypeptide having the activity of a polypeptide having the sequence set forth in SEQ ID No: 4 or SEQ ID No: 11 or a functional fragment of said polypeptide; at least one exogenous nucleic acid encoding a polypeptide having the activity of a polypeptide having the sequence set forth in SEQ ID No: 5 or SEQ ID No: 12 or a functional fragment of said polypeptide; at least one exogenous nucleic acid encoding a polypeptide having the activity of a polypeptide having the sequence set forth in SEQ ID No: 6 or SEQ ID No: 13 or a functional fragment of said polypeptide; at least one exogenous nucleic acid encoding a polypeptide having the activity of a polypeptide having the sequence set forth in SEQ ID No: 7 or SEQ ID No: 14 or a functional fragment of said polypeptide; and at least one exogenous nucleic acid encoding a polypeptide having the activity of a polypeptide having the sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding a polypeptide having the sequence set forth in SEQ ID No: 1 or SEQ ID No: 9 or a functional fragment of said polypeptide; at least one exogenous nucleic acid encoding a polypeptide having the sequence set forth in SEQ ID No: 2 or SEQ ID No: 10 or a functional fragment of said polypeptide; at least one exogenous nucleic acid encoding a polypeptide having the sequence set forth in SEQ ID No: 3 or SEQ ID No: 9 or a functional fragment of said polypeptide; at least one exogenous nucleic acid encoding a polypeptide having the sequence set forth in SEQ ID No: 4 or SEQ ID No: 11 or a functional fragment of said polypeptide; at least one exogenous nucleic acid encoding a polypeptide having the sequence set forth in SEQ ID No: 5 or SEQ ID No: 12 or a functional fragment of said polypeptide; at least one exogenous nucleic acid encoding a polypeptide having the sequence set forth in SEQ ID No: 6 or SEQ ID No: 13 or a functional fragment of said polypeptide; at least one exogenous nucleic acid encoding a polypeptide having the sequence set forth in SEQ ID No: 7 or SEQ ID No: 14 or a functional fragment of said polypeptide; and at least one exogenous nucleic acid encoding a polypeptide having the sequence set forth in SEQ ID No: 8 or SEQ ID No: 15 or a functional fragment of said polypeptide.

In one embodiment, the non-naturally occurring host capable of producing isoprene via the mevalonate pathway comprises an exogenous nucleic acid encoding a polypeptide having the activity of a polypeptide having the sequence set forth in SEQ ID No: 7 or SEQ ID No: 14 or a functional fragment of said polypeptide.

In one embodiment, the non-naturally occurring host capable of producing isoprene via the mevalonate pathway comprises an exogenous nucleic acid encoding a polypeptide having the sequence set forth in SEQ ID No: 7 or SEQ ID No: 14 or a functional fragment of said polypeptide.

In one embodiment the non-naturally occurring host comprises an exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 7 or SEQ ID No: 14 or a functional fragment of said polypeptide.

In one embodiment the non-naturally occurring host comprises an exogenous nucleic acid encoding a polypeptide having at least 90%, 91%, 92%, 93%, 94%, 95%, 96%, 97%, 98% or 99% sequence identity to the sequence set forth in SEQ ID No: 7 or SEQ ID No: 14 or a functional fragment of said polypeptide.

In one embodiment, the non-naturally occurring host capable of producing isoprene via the mevalonate pathway is capable of expressing a polypeptide having the activity of an isopentenyl diphosphate isomerase enzyme having the amino acid sequence set forth in SEQ ID No: 7 or SEQ ID No: 14 or a functional fragment of said enzyme.

In one embodiment, the non-naturally occurring host capable of producing isoprene via the mevalonate pathway is capable of expressing an isopentenyl diphosphate isomerase enzyme having the amino acid sequence set forth in SEQ ID No: 7 or SEQ ID No: 14 or a functional fragment of said enzyme.

In one embodiment, the non-naturally occurring host capable of producing isoprene via the mevalonate pathway is capable of expressing a polypeptide having the activity of an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme.

In one embodiment, the non-naturally occurring host capable of producing isoprene via the mevalonate pathway is capable of expressing an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme.

In one embodiment, the non-naturally occurring host capable of producing isoprene via the mevalonate pathway comprises at, least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 1 or SEQ ID No: 9 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 2 or SEQ ID No: 10 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 3 or SEQ ID No: 9 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 4 or SEQ ID No: 11 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 5 or SEQ ID No: 12 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 6 or SEQ ID No: 13 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 7 or SEQ ID No: 14 or a functional fragment of said polypeptide; and at least one exogenous nucleic acid encoding a polypeptide having the activity of a polypeptide having at least 90%, 91%, 92%, 93%, 94%, 95%, 96%, 97%, 98% or 99% sequence identity to the sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding a polypeptide having at least 90%, 91%, 92%, 93%, 94%, 95%, 96%, 97%, 98% or 99% sequence identity to the sequence set forth in SEQ ID No: 1 or SEQ ID No: 9 or a functional fragment of said polypeptide; at least one exogenous nucleic acid encoding a polypeptide having at least 90%, 91%, 92%, 93%, 94%, 95%, 96%, 97%, 98% or 99% sequence identity to the sequence set forth in SEQ ID No: 2 or SEQ ID No: 10 or a functional fragment of said polypeptide; at least one exogenous nucleic acid encoding a polypeptide having at least 90%, 91%, 92%, 93%, 94%, 95%, 96%, 97%, 98% or 99% sequence identity to the sequence set forth in SEQ ID No: 3 or SEQ ID No: 9 or a functional fragment of said polypeptide; at least one exogenous nucleic acid encoding a polypeptide having at least 90%, 91%, 92%, 93%, 94%, 95%, 96%, 97%, 98% or 99% sequence identity to the sequence set forth in SEQ ID No: 4 or SEQ ID No: 11 or a functional fragment of said polypeptide; at least one exogenous nucleic acid encoding a polypeptide having at least 90%, 91%, 92%, 93%, 94%, 95%, 96%, 97%, 98% or 99% sequence identity to the sequence set forth in SEQ ID No: 5 or SEQ ID No: 12 or a functional fragment of said polypeptide; at least one exogenous nucleic acid encoding a polypeptide having at least 90%, 91%, 92%, 93%, 94%, 95%, 96%, 97%, 98% or 99% sequence identity to the sequence set forth in SEQ ID No: 6 or SEQ ID No: 13 or a functional fragment of said polypeptide; at least one exogenous nucleic acid encoding a polypeptide having at least 90%, 91%, 92%, 93%, 94%, 95%, 96%, 97%, 98% or 99% sequence identity to the sequence set forth in SEQ ID No: 7 or SEQ ID No: 14 or a functional fragment of said polypeptide; and at least one exogenous nucleic acid encoding a polypeptide having at least 90%, 91%, 92%, 93%, 94%, 95%, 96%, 97%, 98% or 99% sequence identity to the sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding a polypeptide having the activity of a polypeptide having the sequence set forth in SEQ ID No: 1 or SEQ ID No: 9 or a functional fragment of said polypeptide; at least one exogenous nucleic acid encoding a polypeptide having the activity of a polypeptide having the sequence set forth in SEQ ID No: 2 or SEQ ID No: 10 or a functional fragment of said polypeptide; at least one exogenous nucleic acid encoding a polypeptide having the activity of a polypeptide having the sequence set forth in SEQ ID No: 3 or SEQ ID No: 9 or a functional fragment of said polypeptide; at least one exogenous nucleic acid encoding a polypeptide having the activity of a polypeptide having the sequence set forth in SEQ ID No: 4 or SEQ ID No: 11 or a functional fragment of said polypeptide; at least one exogenous nucleic acid encoding a polypeptide having the activity of a polypeptide having the sequence set forth in SEQ ID No: 5 or SEQ ID No: 12 or a functional fragment of said polypeptide; at least one exogenous nucleic acid encoding a polypeptide having the activity of a polypeptide having the sequence set forth in SEQ ID No: 6 or SEQ ID No: 13 or a functional fragment of said polypeptide; at least one exogenous nucleic acid encoding a polypeptide having the activity of a polypeptide having the sequence set forth in SEQ ID No: 7 or SEQ ID No: 14 or a functional fragment of said polypeptide; and at least one exogenous nucleic acid encoding a polypeptide having the activity of a polypeptide having at least 90%, 91%, 92%, 93%, 94%, 95%, 96%, 97%, 98% or 99% sequence identity to the nucleic acid sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding a polypeptide having the sequence set forth in SEQ ID No: 1 or SEQ ID No: 9 or a functional fragment of said polypeptide; at least one exogenous nucleic acid encoding a polypeptide having the sequence set forth in SEQ ID No: 2 or SEQ ID No: 10 or a functional fragment of said polypeptide; at least one exogenous nucleic acid encoding a polypeptide having the sequence set forth in SEQ ID No: 3 or SEQ ID No: 9 or a functional fragment of said polypeptide; at least one exogenous nucleic acid encoding a polypeptide having the sequence set forth in SEQ ID No: 4 or SEQ ID No: 11 or a functional fragment of said polypeptide; at least one exogenous nucleic acid encoding a polypeptide having the sequence set forth in SEQ ID No: 5 or SEQ ID No: 12 or a functional fragment of said polypeptide; at least one exogenous nucleic acid encoding a polypeptide having the sequence set forth in SEQ ID No: 6 or SEQ ID No: 13 or a functional fragment of said polypeptide; at least one exogenous nucleic acid encoding a polypeptide having the sequence set forth in SEQ ID No: 7 or SEQ ID No: 14 or a functional fragment of said polypeptide; and at least one exogenous nucleic acid encoding a polypeptide having at least 90%, 91%, 92%, 93%, 94%, 95%, 96%, 97%, 98% or 99% sequence identity to the nucleic acid sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding a polypeptide having the activity of a polypeptide having the sequence set forth in SEQ ID No: 1 or SEQ ID No: 9 or a functional fragment of said polypeptide; at least one exogenous nucleic acid encoding a polypeptide having the activity of a polypeptide having the sequence set forth in SEQ ID No: 2 or SEQ ID No: 10 or a functional fragment of said polypeptide; at least one exogenous nucleic acid encoding a polypeptide having the activity of a polypeptide having the sequence set forth in SEQ ID No: 3 or SEQ ID No: 9 or a functional fragment of said polypeptide; at least one exogenous nucleic acid encoding a polypeptide having the activity of a polypeptide having the sequence set forth in SEQ ID No: 4 or SEQ ID No: 11 or a functional fragment of said polypeptide; at least one exogenous nucleic acid encoding a polypeptide having the activity of a polypeptide having the sequence set forth in SEQ ID No: 5 or SEQ ID No: 12 or a functional fragment of said polypeptide; at least one exogenous nucleic acid encoding a polypeptide having the activity of a polypeptide having the sequence set forth in SEQ ID No: 6 or SEQ ID No: 13 or a functional fragment of said polypeptide; at least one exogenous nucleic acid encoding a polypeptide having the activity of a polypeptide having at least 90%, 91%, 92%, 93%, 94%, 95%, 96%, 97%, 98% or 99% sequence identity to the nucleic acid sequence set forth in SEQ ID No: 7 or SEQ ID No: 14; and at least one exogenous nucleic acid encoding a polypeptide having the activity of a polypeptide having the sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding a polypeptide having the sequence set forth in SEQ ID No: 1 or SEQ ID No: 9 or a functional fragment of said polypeptide; et least one exogenous nucleic acid encoding a polypeptide having the sequence set forth in SEQ ID No: 2 or SEQ ID No: 10 or a functional fragment of said polypeptide; at least one exogenous nucleic acid encoding a polypeptide having the sequence set forth in SEQ ID No: 3 or SEQ ID No: 9 or a functional fragment of said polypeptide; at least one exogenous nucleic acid encoding a polypeptide having the sequence set forth in SEQ ID No: 4 or SEQ ID No: 11 or a functional fragment of said polypeptide; at least one exogenous nucleic acid encoding a polypeptide having the sequence set forth in SEQ ID No: 5 or SEQ ID No: 12 or a functional fragment of said polypeptide; at least one exogenous nucleic acid encoding a polypeptide having the sequence set forth in SEQ ID No: 6 or SEQ ID No: 13 or a functional fragment of said polypeptide; at least one exogenous nucleic acid encoding a polypeptide having at least 90%, 91%, 92%, 93%, 94%, 95%, 96%, 97%, 98% or 99% sequence identity to the nucleic acid sequence set forth in SEQ ID No: 7 or SEQ ID No: 14; and at least one exogenous nucleic acid encoding a polypeptide having the sequence set forth in SEQ ID No: 8 or SEQ ID No: 15 or a functional fragment of said polypeptide.

In one embodiment, the non-naturally occurring host capable of producing isoprene via the mevalonate pathway comprises at least one exogenous nucleic acid encoding encoding a polypeptide having the activity of an acetyl-CoA acetyltransferase enzyme having the amino acid sequence set forth in SEQ ID No: 1 or SEQ ID No: 9 or a functional fragment of said enzyme; at least one exogenous nucleic acid encoding encoding a polypeptide having the activity of a hydroxymethylglutaryl-CoA synthase enzyme having the amino acid sequence set forth in SEQ ID No: 2 or SEQ ID No: 10 or a functional fragment of said enzyme; at least one exogenous nucleic acid encoding encoding a polypeptide having the activity of a hydroxymethylglutaryl Co-A reductase enzyme having the amino acid sequence set forth in SEQ ID No: 3 or SEQ ID No: 9 or a functional fragment of said enzyme; at least one exogenous nucleic acid encoding encoding a polypeptide having the activity of a mevalonate-kinase enzyme having the amino acid sequence set forth in SEQ ID No: 4 or SEQ ID No: 11 or a functional fragment of said enzyme; at least one exogenous nucleic acid encoding encoding a polypeptide having the activity of a phosphomevalonate kinase enzyme having the amino acid sequence set forth in SEQ ID No: 5 or SEQ ID No: 12 or a functional fragment of said enzyme; at least one exogenous nucleic acid encoding encoding a polypeptide having the activity of a diphosphomevalonate decarboxylase enzyme having the amino acid sequence set forth in SEQ ID No: 6 or SEQ ID No: 13 or a functional fragment of said enzyme; at least one exogenous nucleic acid encoding encoding a polypeptide having the activity of an isopentenyl diphosphate isomerase enzyme having the amino acid sequence set forth in SEQ ID No: 7 or SEQ ID No: 14 or a functional fragment of said enzyme; and at least one exogenous nucleic acid encoding encoding a polypeptide having the activity of an isoprene synthase enzyme having the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding an acetyl-CoA acetyltransferase enzyme having the amino acid sequence set forth in SEQ ID No: 1 or SEQ ID No: 9 or a functional fragment of said enzyme; at least one exogenous nucleic acid encoding a hydroxymethylglutaryl-CoA synthase enzyme having the amino acid sequence set forth in SEQ ID No: 2 or SEQ ID No: 10 or a functional fragment of said enzyme; at least one exogenous nucleic acid encoding a hydroxymethylglutaryl Co-A reductase enzyme having the amino acid sequence set forth in SEQ ID No: 3 or SEQ ID No: 9 or a functional fragment of said enzyme; at least one exogenous nucleic acid encoding a mevalonate-kinase enzyme having the amino acid sequence set forth in SEQ ID No: 4 or SEQ ID No: 11 or a functional fragment of said enzyme; at least one exogenous nucleic acid encoding a phosphomevalonate kinase enzyme having the amino acid sequence set forth in SEQ ID No: 5 or SEQ ID No: 12 or a functional fragment of said enzyme; at least one exogenous nucleic acid encoding a diphosphomevalonate decarboxylase enzyme having the amino acid sequence set forth in SEQ ID No: 6 or SEQ ID No: 13 or a functional fragment of said enzyme; at least one exogenous nucleic acid encoding an isopentenyl diphosphate isomerase enzyme having the amino acid sequence set forth in SEQ ID No: 7 or SEQ ID No: 14 or a functional fragment of said enzyme; and at least one exogenous nucleic acid encoding an isoprene synthase enzyme having the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding a polypeptide having the activity of an acetyl-CoA 95%, 96%, 97%, acetyltransferase enzyme having at least 90%, 91%, 92%, 93%, 94%, 98% or 99% sequence identity to the amino acid sequence set forth in SEQ ID No: 1 or SEQ ID No: 9 or a functional fragment of said enzyme; at least one exogenous nucleic acid encoding a polypeptide having the activity of a hydroxymethylglutaryl-CoA synthase enzyme having at least 90%, 91%, 92%, 93%, 94%, 95%, 96%, 97%, 98% or 99% sequence identity to the amino acid sequence set forth in SEQ ID No: 2 or SEQ ID No: 10 or a functional fragment of said enzyme; at least one exogenous nucleic acid encoding a polypeptide having the activity of a hydroxymethylglutaryl Co-A reductase enzyme having at least 90%, 91%, 92%, 93%, 94%, 95%, 96%, 97%, 98% or 99% sequence identity to the amino acid sequence set forth in SEQ ID No: 3 or SEQ ID No: 9 or a functional fragment of said enzyme; at least one exogenous nucleic acid encoding a polypeptide having the activity of a mevalonate-kinase enzyme having at least 90%, 91%, 92%, 93%, 94%, 95%, 96%, 97%, 98% or 99% sequence identity to the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having at least 90%, 91%, 92%, 93%, 94%, 95%, 96%, 97%, 98% or 99% sequence identity to the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having at least 90%, 91%, 92%, 93%, 94%, 95%, 96%, 97%, 98% or 99% sequence identity to the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and at least one exogenous nucleic acid encoding a polypeptide having the activity of an isoprene synthase enzyme having at least 90%, 91%, 92%, 93%, 94%, 95%, 96%, 97%, 98% or 99% sequence identity to the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding an acetyl-CoA acetyltransferase enzyme having at least 90%, 91%, 92%, 93%, 94%, 95%, 96%, 97%, 98% or 99% sequence identity to the amino acid sequence set forth in SEQ ID No: 1 or SEQ ID No: 9 or a functional fragment of said enzyme; at least one exogenous nucleic acid encoding a hydroxymethylglutaryl-CoA synthase enzyme having at least 90%, 91%, 92%, 93%, 94%, 95%, 96%, 97%, 98% or 99% sequence identity to the amino acid sequence set forth in SEQ ID No: 2 or SEQ ID No: 10 or a functional fragment of said enzyme; at least one exogenous nucleic acid encoding a hydroxymethylglutaryl Co-A reductase enzyme having at least 90%, 91%, 92%, 93%, 94%, 95%, 96%, 97%, 98% or 99% sequence identity to the amino acid sequence set forth in SEQ ID No: 3 or SEQ ID No: 9 or a functional fragment of said enzyme; at least one exogenous nucleic acid encoding a mevalonate-kinase enzyme having at least 90%, 91%, 92%, 93%, 94%, 95%, 96%, 97%, 98% or 99% sequence identity to the amino acid sequence set forth in SEQ ID No: 4 or SEQ ID No: 11 or a functional fragment of said enzyme; at least one exogenous nucleic acid encoding a phosphomevalonate kinase enzyme having at least 90%, 91%, 92%, 93%, 94%, 95%, 96%, 97%, 98% or 99% sequence identity to the amino acid sequence set forth in SEQ ID No: 5 or SEQ ID No: 12 or a functional fragment of said enzyme; at least one exogenous nucleic acid encoding a diphosphomevalonate decarboxylase enzyme having at least 90%, 91%, 92%, 93%, 94%, 95%, 96%, 97%, 98% or 99% sequence identity to the amino acid sequence set forth in SEQ ID No: 6 or SEQ ID No: 13 or a functional fragment of said enzyme; at least one exogenous nucleic acid encoding an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and at least one exogenous nucleic acid encoding an isoprene synthase enzyme having at least 90%, 91%, 92%, 93%, 94%, 95%, 96%, 97%, 98% or 99% sequence identity to the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding a polypeptide having the activity of an acetyl-CoA acetyltransferase enzyme having the amino acid sequence set forth in SEQ ID No: 1 or SEQ ID No: 9 or a functional fragment of said enzyme; at least one exogenous nucleic acid encoding a polypeptide having the activity of a hydroxymethylglutaryl-CoA synthase enzyme having the amino acid sequence set forth in SEQ ID No: 2 or SEQ ID No: 10 or a functional fragment of said enzyme; at least one exogenous nucleic acid encoding a polypeptide having the activity of a hydroxymethylglutaryl Co-A reductase enzyme having the amino acid sequence set forth in SEQ ID No: 3 or SEQ ID No: 9 or a functional fragment of said enzyme; at least one exogenous nucleic acid encoding a polypeptide having the activity of a mevalonate-kinase enzyme having the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having the amino acid sequence set forth in SEQ ID No: 7 or SEQ ID No: 14 or a functional fragment of said enzyme; and at least one exogenous nucleic acid encoding a polypeptide having the activity of an isoprene synthase enzyme classified under EC 4.2.3.27 or a functional fragment of said enzyme, for example an isoprene synthase having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding an acetyl-CoA acetyltransferase enzyme having the amino acid sequence set forth in SEQ ID No: 1 or SEQ ID No: 9 or a functional fragment of said enzyme; at least one exogenous nucleic acid encoding a hydroxymethylglutaryl-CoA synthase enzyme having the amino acid sequence set forth in SEQ ID No: 2 or SEQ ID No: 10 or a functional fragment of said enzyme; at least one exogenous nucleic acid encoding a hydroxymethylglutaryl Co-A reductase enzyme having the amino acid sequence set forth in SEQ ID No: 3 or SEQ ID No: 9 or a functional fragment of said enzyme; at least one exogenous nucleic acid encoding a mevalonate-kinase enzyme having the amino acid sequence set forth in SEQ ID No: 4 or SEQ ID No: 11 or a functional fragment of said enzyme; at least one exogenous nucleic acid encoding a phosphomevalonate kinase enzyme having the amino acid sequence set forth in SEQ ID No: 5 or SEQ ID No: 12 or a functional fragment of said enzyme; at least one exogenous nucleic acid encoding a diphosphomevalonate decarboxylase enzyme having the amino acid sequence set forth in SEQ ID No: 6 or SEQ ID No: 13 or a functional fragment of said enzyme; at least one exogenous nucleic acid encoding an isopentenyl diphosphate isomerase enzyme having the amino acid sequence set forth in SEQ ID No: 7 or SEQ ID No: 14 or a functional fragment of said enzyme; and at least one exogenous nucleic acid encoding an isoprene synthase enzyme classified under EC 4.2.3.27 or a functional fragment of said enzyme, for example an isoprene synthase having at least 90%, 91%, 92%, 93%, 94%, 95%, 96%, 97%, 98%, or 99% sequence identity to the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding a polypeptide having the activity of an acetyl-CoA acetyltransferase enzyme having the amino acid sequence set forth in SEQ ID No: 1 or SEQ ID No: 9 or a functional fragment of said enzyme; at least one exogenous nucleic acid encoding a polypeptide having the activity of a hydroxymethylglutaryl-CoA synthase enzyme having the amino acid sequence set forth in SEQ ID No: 2 or SEQ ID No: 10 or a functional fragment of said enzyme; at least one exogenous nucleic acid encoding a polypeptide having the activity of a hydroxymethylglutaryl Co-A reductase enzyme having the amino acid sequence set forth in SEQ ID No: 3 or SEQ ID No: 9 or a functional fragment of said enzyme; at least one exogenous nucleic acid encoding a polypeptide having the activity of a mevalonate-kinase enzyme having the amino acid sequence set forth in SEQ ID No: 4 or SEQ ID No: 11 or a functional fragment of said enzyme; at least one exogenous nucleic acid encoding a polypeptide having the activity of a phosphomevalonate kinase enzyme having the amino acid sequence set forth in SEQ ID No: 5 or SEQ ID No: 12 or a functional fragment of said enzyme; at least one exogenous nucleic acid encoding a polypeptide having the activity of a diphosphomevalonate decarboxylase enzyme having the amino acid sequence set forth in SEQ ID No: 6 or SEQ ID No: 13 or a functional fragment of said enzyme; at least one exogenous nucleic acid encoding a polypeptide having the activity of an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and at least one exogenous nucleic acid encoding a polypeptide having the activity of an isoprene synthase enzyme having the amino acid sequence set forth in SEQ ID No: 8 or SEQ ID No: 15 or a functional fragment of said enzyme.

In one embodiment, the non-naturally occurring host capable of producing isoprene via the mevalonate pathway comprises at least one exogenous nucleic acid encoding an acetyl-CoA acetyltransferase enzyme having the amino acid sequence set forth in SEQ ID No: 1 or SEQ ID No: 9 or a functional fragment of said enzyme; at least one exogenous nucleic acid encoding a hydroxymethylglutaryl-CoA synthase enzyme having the amino acid sequence set forth in SEQ ID No: 2 or SEQ ID No: 10 or a functional fragment of said enzyme; at least one exogenous nucleic acid encoding a hydroxymethylglutaryl Co-A reductase enzyme having the amino acid sequence set forth in SEQ ID No: 3 or SEQ ID No: 9 or a functional fragment of said enzyme; at least one exogenous nucleic acid encoding a mevalonate-kinase enzyme having the amino acid sequence set forth in SEQ ID No: 4 or SEQ ID No: 11 or a functional fragment of said enzyme; at least one exogenous nucleic acid encoding a phosphomevalonate kinase enzyme having the amino acid sequence set forth in SEQ ID No: 5 or SEQ ID No: 12 or a functional fragment of said enzyme; at least one exogenous nucleic acid encoding a diphosphomevalonate decarboxylase enzyme having the amino acid sequence set forth in SEQ ID No: 6 or SEQ ID No: 13 or a functional fragment of said enzyme; at least one exogenous nucleic acid encoding an isopentenyl diphosphate isomerase enzyme having at least 90%, 91%, 92%, 93%, 94%, 95%, 96%, 97%, 98% or 99% sequence identity to the amino acid sequence set forth in SEQ ID No: 7 or SEQ ID No: 14 or a functional fragment of said enzyme; and at least one exogenous nucleic acid encoding an isoprene synthase enzyme having the amino acid sequence set forth in SEQ ID No: 8 or SEQ ID No: 15 or a functional fragment of said enzyme.

In one embodiment, the methods described herein are performed in a non-naturally occurring host, for example a prokaryotic or eukaryotic host.

In one embodiment, at least one of the enzymatic conversions of the methods described herein is performed in a non-naturally occurring host, for example a prokaryotic or eukaryotic host.

In one embodiment, the non-naturally occurring host is a prokaryotic host.

In one embodiment, the non-naturally occurring host is a prokaryotic host from the genus Escherichia, Clostridia, Corynebacteria, Cupriavidus, Pseudomonas, Bacillus, or Rhodococcus.

In one embodiment, the non-naturally occurring host is from the genus Cupriavidus.

In one embodiment, the non-naturally occurring host is Cupriavidus necator.

In one embodiment, the non-naturally occurring host is a eukaryotic host.

In one embodiment, the non-naturally occurring host is a eukaryotic host from the genus Aspergillus, Saccharomyces, Pichia, Yarrowia, Issatchenkia, Debaryomyces, Arxula, or Kluyveromyces.

In one embodiment, the non-naturally occurring host is capable of endogenously producing isoprene via a non-mevalonate pathway.

In one embodiment, at least one of the enzymatic conversions of the method for synthesizing isoprene via the mevalonate pathway comprises gas fermentation, for example gas fermentation wherein the gas comprises at least one of natural gas, syngas, CO₂/H₂, methanol, ethanol, non-volatile residue, caustic wash from cyclohexane oxidation processes, or waste stream from a chemical or petrochemical industry. In one embodiment, the gas is CO₂/H₂.

In one embodiment, the method for synthesizing isoprene via the mevalonate pathway comprises culturing a non-naturally occurring host described herein in a gas medium.

In one embodiment, the method for synthesizing isoprene via the mevalonate pathway comprises culturing a non-naturally occurring host described herein in a gas medium and recovering the produced isoprene.

In one embodiment, the non-naturally occurring host capable of producing isoprene via the mevalonate pathway performs the enzymatic synthesis by gas fermentation, for example gas fermentation wherein the gas comprises at least one of natural gas, syngas, CO₂/H₂, methanol, ethanol, non-volatile residue, caustic wash from cyclohexane oxidation processes, or waste stream from a chemical or petrochemical industry. In one embodiment, the gas is CO₂/H₂.

In one embodiment is provided a method for synthesizing dimethyldiallyl diphosphate.

In one embodiment, the method for synthesizing dimethyldiallyl diphosphate comprises: enzymatically converting (R)-mevalonate to (R)-5-phosphomevalonate using a polypeptide having the activity of a mevalonate-kinase enzyme having the amino acid sequence set forth in SEQ ID 11 or a functional fragment of said enzyme; enzymatically converting (R)-5-phosphomevalonate to (R)-5-diphosphomevalonate using a polypeptide having the activity of a phosphomevalonate kinase enzyme having the amino acid sequence set forth in SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having the amino acid sequence set forth in SEQ ID No: 13 or a functional fragment of said enzyme; and enzymatically converting isopentenyl diphosphate to dimethylallyl diphosphate using a polypeptide having the activity of an isopentenyl diphosphate isomerase enzyme having the amino acid sequence set forth in SEQ ID No 14 or a functional fragment of said enzyme.

In one embodiment, the method for synthesizing dimethyldiallyl diphosphate comprises: enzymatically converting (R)-mevalonate to (R)-5-phosphomevalonate using a mevalonate-kinase enzyme having the amino acid sequence set forth in SEQ ID 11 or a functional fragment of said enzyme; enzymatically converting (R)-5-phosphomevalonate to (R)-5-diphosphomevalonate using a phosphomevalonate kinase enzyme having the amino acid sequence set forth in SEQ ID No: 12 or a functional fragment of said enzyme; enzymatically converting (R)-5-diphosphomevalonate to isopentenyl diphosphate using a diphosphomevalonate decarboxylase enzyme having the amino acid sequence set forth in SEQ ID No: 13 or a functional fragment of said enzyme; and enzymatically converting isopentenyl diphosphate to dimethylallyl diphosphate using an isopentenyl diphosphate isomerase enzyme having the amino acid sequence set forth in SEQ ID No 14 or a functional fragment of said enzyme.

In one embodiment, the method for synthesizing dimethyldiallyl diphosphate comprises: enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID 11 or a functional fragment of said enzyme; enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 12 or a functional fragment of said enzyme; enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 13 or a functional fragment of said enzyme; and enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No 14 or a functional fragment of said enzyme.

In one embodiment, the method for synthesizing dimethyldiallyl diphosphate comprises: enzymatically converting (R)-mevalonate to (R)-5-phosphomevalonate using a mevalonate-kinase enzyme having at least 90%, 91%, 92%, 93%, 94%, 95%, 96%, 97%, 98%, or 99% sequence identity to the amino acid sequence set forth in SEQ ID 11 or a functional fragment of said enzyme; enzymatically converting (R)-5-phosphomevalonate to (R)-5-diphosphomevalonate using a phosphomevalonate kinase enzyme having at least 90%, 91%, 92%, 93%, 94%, 95%, 96%, 97%, 98%, or 99% sequence identity to the amino acid sequence set forth in SEQ ID No: 12 or a functional fragment of said enzyme; enzymatically converting (R)-5-diphosphomevalonate to isopentenyl diphosphate using a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 13 or a functional fragment of said enzyme; and enzymatically converting isopentenyl diphosphate to dimethylallyl diphosphate using an isopentenyl diphosphate isomerase enzyme having at least 90%, 91%, 92%, 93%, 94%, 95%, 96%, 97%, 98%, or 99% sequence identity to the amino acid sequence set forth in SEQ ID No 14 or a functional fragment of said enzyme.

In one embodiment is provided a method for synthesizing isoprene comprising enzymatically converting dimethylallyl diphosphate synthesized according to a method described herein to isoprene using a polypeptide having the activity of an isoprene synthase enzyme having the amino acid sequence set forth in SEQ ID No: 15 or a functional fragment of said enzyme.

In one embodiment is provided a method for synthesizing isoprene comprising enzymatically converting dimethylallyl diphosphate synthesized according to a method described herein to isoprene using an isoprene synthase enzyme having the amino acid sequence set forth in SEQ ID No: 15 or a functional fragment of said enzyme.

In one embodiment is provided a method for synthesizing isoprene comprising enzymatically converting dimethylallyl diphosphate synthesized according to a method described herein to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 15 or a functional fragment of said enzyme.

In one embodiment is provided a method for synthesizing isoprene comprising enzymatically converting dimethylallyl diphosphate synthesized according to a method described herein to isoprene using an isoprene synthase enzyme having at least 90%, 91%, 92%, 93%, 94%, 95%, 96%, 97%, 98%, or 99% sequence identity to the amino acid sequence set forth in SEQ ID No: 15 or a functional fragment of said enzyme.

In one embodiment, the method for synthesizing dimethyldiallyl diphosphate is performed in a recombinant host, for example from the genus Cupriavidus, for example Cupriavidus necator.

In one embodiment is provided a non-naturally occurring chemolithotrophic host capable of producing dimethylallyl diphosphate via the lower mevalonate pathway.

In one embodiment, the non-naturally occurring chemolithotrophic host capable of producing dimethylallyl diphosphate via the lower mevalonate pathway comprises: at least one exogenous nucleic acid encoding a polypeptide having the activity of a mevalonate-kinase enzyme having the amino acid sequence set forth in SEQ ID No: 11 or a functional fragment of said enzyme; at least one exogenous nucleic acid encoding a polypeptide having the activity of a phosphomevalonate kinase enzyme having the amino acid sequence set forth in SEQ ID No: 12 or a functional fragment of said enzyme; at least one exogenous nucleic acid encoding a polypeptide having the activity of a diphosphomevalonate decarboxylase enzyme having the amino acid sequence set forth in SEQ ID No: 13 or a functional fragment of said enzyme; and at least one exogenous nucleic acid encoding a polypeptide having the activity of an isopentenyl diphosphate isomerase enzyme having the amino acid sequence set forth in SEQ ID No: 14 or a functional fragment of said enzyme.

In one embodiment, the non-naturally occurring chemolithotrophic host capable of producing dimethylallyl diphosphate via the lower mevalonate pathway comprises: at least one exogenous nucleic acid encoding a mevalonate-kinase enzyme having the amino acid sequence set forth in SEQ ID No: 11 or a functional fragment of said enzyme; at least one exogenous nucleic acid encoding a phosphomevalonate kinase enzyme having the amino acid sequence set forth in SEQ ID No: 12 or a functional fragment of said enzyme; at least one exogenous nucleic acid encoding a diphosphomevalonate decarboxylase enzyme having the amino acid sequence set forth in SEQ ID No: 13 or a functional fragment of said enzyme; and at least one exogenous nucleic acid encoding an isopentenyl diphosphate isomerase enzyme having the amino acid sequence set forth in SEQ ID No: 14 or a functional fragment of said enzyme.

In one embodiment, the non-naturally occurring chemolithotrophic host capable of producing dimethylallyl diphosphate via the lower mevalonate pathway comprises: at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 11 or a functional fragment of said enzyme; at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 12 or a functional fragment of said enzyme; at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 13 or a functional fragment of said enzyme; and at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 14 or a functional fragment of said enzyme.

In one embodiment, the non-naturally occurring chemolithotrophic host capable of producing dimethylallyl diphosphate via the lower mevalonate pathway comprises: at least one exogenous nucleic acid encoding a mevalonate-kinase enzyme having at least 90%, 91%, 92%, 93%, 94%, 95%, 96%, 97%, 98%, or 99% sequence identity to the amino acid sequence set forth in SEQ ID No: 11 or a functional fragment of said enzyme; at least one exogenous nucleic acid encoding a phosphomevalonate kinase enzyme having at least 90%, 91%, 92%, 93%, 94%, 95%, 96%, 97%, 98%, or 99% sequence identity to the amino acid sequence set forth in SEQ ID No: 12 or a functional fragment of said enzyme; at least one exogenous nucleic acid encoding a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 13 or a functional fragment of said enzyme; and at least one exogenous nucleic acid encoding an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 14 or a functional fragment of said enzyme.

In one embodiment, the non-naturally occurring chemolithotrophic host capable of producing dimethylallyl diphosphate via the lower mevalonate pathway further comprises comprises at least one exogenous nucleic acid encoding a polypeptide having the activity of an isoprene synthase enzyme having the amino acid sequence set forth in SEQ ID No: 15 or a functional fragment of said enzyme.

In one embodiment, the non-naturally occurring chemolithotrophic host capable of producing dimethylallyl diphosphate via the lower mevalonate pathway further comprises comprises at least one exogenous nucleic acid encoding an isoprene synthase enzyme having the amino acid sequence set forth in SEQ ID No: 15 or a functional fragment of said enzyme.

In one embodiment, the non-naturally occurring chemolithotrophic host capable of producing dimethylallyl diphosphate via the lower mevalonate pathway further comprises comprises at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 15 or a functional fragment of said enzyme.

In one embodiment, the non-naturally occurring chemolithotrophic host capable of producing dimethylallyl diphosphate via the lower mevalonate pathway further comprises comprises at least one exogenous nucleic acid encoding an isoprene synthase enzyme having at least 90%, 91%, 92%, 93%, 94%, 95%, 96%, 97%, 98%, or 99% sequence identity to the amino acid sequence set forth in SEQ ID No: 15 or a functional fragment of said enzyme.

In one embodiment, the non-naturally occurring chemolithotrophic host capable of producing dimethylallyl diphosphate via the lower mevalonate pathway is from the genus Cupriavidus, for example Cupriavidus necator.

In one embodiment, the non-naturally occurring chemolithotrophic host capable of producing dimethylallyl diphosphate via the lower mevalonate pathway comprises a plasmid.

In one embodiment is provided a method for synthesizing mevalonate in a chemolithotrophic host comprising: enzymatically converting acetyl-CoA to acetoacetyl-CoA using a polypeptide having the activity of an acetoacetyl-CoA C-acetyltransferase enzyme having the amino acid sequence set forth in SEQ ID No: 9 or a functional fragment of said enzyme; enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 10 or a functional fragment of said enzyme; and enzymatically converting 3-hydroxy-3-methylglutaryl-CoA to (R)-mevalonate using a polypeptide having the activity of a hydroxymethylglutaryl Co-A′reductase enzyme having the amino acid sequence set forth in SEQ ID No: 9 or a functional fragment of said enzyme.

In one embodiment is provided a method for synthesizing mevalonate in a chemolithotrophic host comprising: enzymatically converting acetyl-CoA to acetoacetyl-CoA using an acetoacetyl-CoA C-acetyltransferase enzyme having the amino acid sequence set forth in SEQ ID No: 9 or a functional fragment of said enzyme; enzymatically converting acetoacetyl-CoA to 3-hydroxy-3-methylglutaryl-CoA using a hydroxymethylglutaryl-CoA synthase enzyme having the amino acid sequence set forth in SEQ ID No: 10 or a functional fragment of said enzyme; and enzymatically converting 3-hydroxy-3-methylglutaryl-CoA to (R)-mevalonate using a hydroxymethylglutaryl Co-A reductase enzyme having the amino acid sequence set forth in SEQ ID No: 9 or a functional fragment of said enzyme.

In one embodiment is provided a method for synthesizing mevalonate in a chemolithotrophic host comprising: enzymatically converting acetyl-CoA to acetoacetyl-CoA using a polypeptide having the activity of an acetoacetyl-CoA C-acetyltransferase enzyme having at least 90%, 91%, 92%, 93%, 94%, 95%, 96%, 97%, 98%, or 99% sequence identity to the amino acid sequence set forth in SEQ ID No: 9 or a functional fragment of said enzyme; enzymatically converting acetoacetyl-CoA to 3-hydroxy-3-methylglutaryl-CoA using a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 10 or a functional fragment of said enzyme; and enzymatically converting 3-hydroxy-3-methylglutaryl-CoA to (R)-mevalonate using a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 9 or a functional fragment of said enzyme.

In one embodiment is provided a method for synthesizing mevalonate in a chemolithotrophic host comprising: enzymatically converting acetyl-CoA to acetoacetyl-CoA using an acetoacetyl-CoA C-acetyltransferase enzyme having at least 90%, 91%, 92%, 93%, 94%, 95%, 96%, 97%, 98%, or 99% sequence identity to the amino acid sequence set forth in SEQ ID No: 9 or a functional fragment of said enzyme; enzymatically converting acetoacetyl-CoA to 3-hydroxy-3-methylglutaryl-CoA using a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 10 or a functional fragment of said enzyme; and enzymatically converting 3-hydroxy-3-methylglutaryl-CoA to (R)-mevalonate using a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 9 or a functional fragment of said enzyme.

In one embodiment, the method for synthesizing mevalonate is performed in a recombinant host, for example from the genus Cupriavidus, for example Cupriavidus necator.

In one embodiment is provided a non-naturally occurring chemolithotrophic host capable of producing mevalonate via the upper mevalonate pathway.

In one embodiment, the naturally occurring chemolithotrophic host capable of producing mevalonate via the upper mevalonate pathway comprises: at least one exogenous nucleic acid encoding a polypeptide having the activity of an enzyme having the amino acid sequence set forth in SEQ ID No: 9 or a functional fragment of said enzyme; and at least one exogenous nucleic acid encoding a polypeptide having the activity of a hydroxymethylglutaryl-CoA synthase enzyme having the amino acid sequence set forth in SEQ ID No: 10 or a functional fragment of said enzyme.

In one embodiment, the naturally occurring chemolithotrophic host capable of producing mevalonate via the upper mevalonate pathway comprises: at least one exogenous nucleic acid encoding an enzyme having the amino acid sequence set forth in SEQ ID No: 9 or a functional fragment of said enzyme; and at least one exogenous nucleic acid encoding a hydroxymethylglutaryl-CoA synthase enzyme having the amino acid sequence set forth in SEQ ID No: 10 or a functional fragment of said enzyme.

In one embodiment, the naturally occurring chemolithotrophic host capable of producing mevalonate via the upper mevalonate pathway comprises: at least one exogenous nucleic acid encoding a polypeptide having the activity of an enzyme having at least 90%, 91%, 92%, 93%, 94%, 95%, 96%, 97%, 98%, or 99% sequence identity to the amino acid sequence set forth in SEQ ID No: 9 or a functional fragment of said enzyme; and at least one exogenous nucleic acid encoding a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 10 or a functional fragment of said enzyme.

In one embodiment, the naturally occurring chemolithotrophic host capable of producing mevalonate via the upper mevalonate pathway comprises: at least one exogenous nucleic acid encoding an enzyme having at least 90%, 91%, 92%, 93%, 94%, 95%, 96%, 97%, 98%, or 99% sequence identity to the amino acid sequence set forth in SEQ ID No: 9 or a functional fragment of said enzyme; and at least one exogenous nucleic acid encoding a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 10 or a functional fragment of said enzyme.

In one embodiment, the non-naturally occurring chemolithotrophic host capable of producing mevalonate via the upper mevalonate pathway is from the genus Cupriavidus, for example Cupriavidus necator.

In one embodiment, the non-naturally occurring chemolithotrophic host capable of producing mevalonate via the upper mevalonate pathway comprises a plasmid.

In one embodiment is provided a non-naturally occurring mutant or variant of SEQ ID No: 7 or SEQ ID No: 14 or comprising one or more non-naturally-occurring mutations, wherein the mutant or variant exhibits isopentenyl diphosphate isomerase activity.

In one embodiment, at least one enzyme used in a method described herein has an amino acid sequence having at least 70%, 75%, 80%, 85%, 86%, 87%, 88%, or 89% identity to a sequence set forth in any one of the SEQ ID Nos.

In one embodiment is provided a composition comprising a method or host described herein.

In one embodiment is provided isoprene synthesized by a method described herein.

In one embodiment is provided a composition comprising an isopentenyl diphosphate isomerase enzyme having the amino acid sequence set forth in SEQ ID No: 7 or SEQ ID No: 14 or having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme, and further means for enzymatically producing isoprene from a suitable substrate.

In one embodiment is provided a composition comprising a substrate, a polypeptide having the activity of an isopentenyl diphosphate isomerase enzyme having the amino acid sequence set forth in SEQ ID No: 7 or SEQ ID No: 14 or having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme, and further means for enzymatically producing isoprene from said substrate.

In one embodiment is provided a composition comprising a substrate, an isopentenyl diphosphate isomerase enzyme having the amino acid sequence set forth in SEQ ID No: 7 or SEQ ID No: 14 or having at least 90%, 91%, 92%, 93%, 94%, 95%, 96%, 97%, 98%, or 99% sequence identity to the amino acid sequence set forth in SEQ ID No: 7 or SEQ ID No: 14 or a functional fragment of said enzyme, and further means for enzymatically producing isoprene from said substrate.

In one embodiment is provided a method for producing bioderived isoprene, comprising culturing or growing a host described herein under conditions and for a sufficient period of time to produce bioderived isoprene.

In one embodiment is provided bioderived isoprene produced in a host described herein, wherein said bioderived isoprene has a carbon-12, carbon-13, and carbon-14 isotope ratio that reflects an atmospheric carbon dioxide uptake source.

In one embodiment is provided a bio-derived, bio-based, or fermentation-derived product produced from any of the methods or non-naturally occurring hosts described herein, wherein the product comprises

i. a composition comprising at least one bio-derived, bio-based or fermentation-derived compound or any combination thereof,

ii. a bio-derived, bio-based or fermentation-derived polymer comprising the bio-derived, bio-based or fermentation-derived composition or compound of i., or any combination thereof,

iii. a bio-derived, bio-based or fermentation-derived cis-polyisoprene rubber, trans-polyisoprene rubber, or liquid polyisoprene rubber, comprising the bio-derived, bio-based or fermentation-derived compound or bio-derived, bio-based or fermentation-derived composition of i. or any combination thereof or the bio-derived, bio-based or fermentation-derived polymer of ii. or any combination thereof,

iv. a molded substance obtained by molding the bio-derived, bio-based or fermentation-derived polymer of ii. or the bio-derived, bio-based or fermentation-derived resin of iii., or any combination thereof,

v. a bio-derived, bio-based or fermentation-derived formulation comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., or bio-derived, bio-based or fermentation-derived molded substance of iv, or any combination thereof, or

vi. a bio-derived, bio-based or fermentation-derived semi-solid or a non-semi-solid stream, comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., bio-derived, bio-based or fermentation-derived formulation of v., or bio-derived, bio-based or fermentation-derived molded substance of iv., or any combination thereof.

### OTHER EMBODIMENTS

It is to be understood that while the invention has been described in conjunction with the detailed description thereof, the foregoing description is intended to illustrate and not limit the scope of the invention. Other aspects, advantages, and modifications are within the scope of the following claims.

What is claimed is:

1. A method for synthesizing isoprene in a chemolithotrophic host comprising:
enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or 10 or a functional fragment of said enzyme; and
enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or 9 or a functional fragment of said enzyme.

2. A method for synthesizing isoprene in a chemolithotrophic host comprising:
enzymatically converting acetoacetyl-CoA to 3-hydroxy-3-methylglutaryl-CoA using a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 2 or 10 or a functional fragment of said enzyme; and
enzymatically converting 3-hydroxy-3-methylglutaryl-CoA to (R)-mevalonate using a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 3 or 9 or a functional fragment of said enzyme.

3. The method of any of claims 1-2, further comprising at least one of:
enzymatically converting acetyl-CoA to acetoacetyl-CoA using a polypeptide having the activity of an acetyl-CoA acetyltransferase enzyme having the amino acid sequence set forth in SEQ ID No: 1 or 9 or a functional fragment of said enzyme;
enzymatically converting (R)-mevalonate to (R)-5-phosphomevalonate using a polypeptide having the activity of a mevalonate-kinase enzyme having the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having the amino acid sequence set forth in SEQ ID No: 8 or 15 or a functional fragment of said enzyme.

4. The method of any of claims 1-2, further comprising at least one of:
enzymatically converting acetyl-CoA to acetoacetyl-CoA using a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or 9 or a functional fragment of said enzyme;
enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

5. The method of any of claims 1-2, further comprising:
enzymatically converting acetyl-CoA to acetoacetyl-CoA using a polypeptide having the activity of an acetyl-CoA acetyltransferase enzyme having the amino acid sequence set forth in SEQ ID No: 1 or 9 or a functional fragment of said enzyme;
enzymatically converting (R)-mevalonate to (R)-5-phosphomevalonate using a polypeptide having the activity of a mevalonate-kinase enzyme having the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having the amino acid sequence set forth in SEQ ID No: 8 or 15 or a functional fragment of said enzyme.

6. The method of any of claims 1-2, further comprising:
enzymatically converting acetyl-CoA to acetoacetyl-CoA using a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or 9 or a functional fragment of said enzyme;
enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

7. A non-naturally occurring chemolithotrophic host capable of producing isoprene via the mevalonate pathway, said host comprising:
at least one exogenous nucleic acid encoding a polypeptide having the activity of a hydroxymethylglutaryl-CoA synthase enzyme having the amino acid sequence set forth in SEQ ID No: 2 or 10 ora functional fragment of said enzyme; and
at least one exogenous nucleic acid encoding a polypeptide having the activity of a hydroxymethylglutaryl Co-A reductase enzyme having the amino acid sequence set forth in SEQ ID No: 3 or 9 a functional fragment of said enzyme.

8. A non-naturally occurring chemolithotrophic host capable of producing isoprene via the mevalonate pathway, said host comprising:
at least one exogenous nucleic acid encoding a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 2 or 10 or a functional fragment of said enzyme; and
at least one exogenous nucleic acid encoding a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 3 or 9 or a functional fragment of said enzyme.

9. The host of any of claims 7-8, further comprising at least one of:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having the amino acid sequence set forth in SEQ ID No: 7 or 14 or a functional fragment of said enzyme; and
at least one exogenous nucleic acid encoding a polypeptide having the activity of an isoprene synthase enzyme having the amino acid sequence set forth in SEQ ID No: 8 or 15 or a functional fragment of said enzyme.

10. The host of any of claims 7-8, further comprising at least one of:
at least one exogenous nucleic acid encoding a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or 9 or a functional fragment of said enzyme;
at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or 11 or a functional fragment of said enzyme;
at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

11. The host of any of claims 7-8, further comprising:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having the amino acid sequence set forth in SEQ ID No: 7 or 14 or a functional fragment of said enzyme; and
at least one exogenous nucleic acid encoding a polypeptide having the activity of an isoprene synthase enzyme having the amino acid sequence set forth in SEQ ID No: 8 or 15 or a functional fragment of said enzyme.

12. The host of any of claims 7-8, further comprising:
at least one exogenous nucleic acid encoding a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or 9 or a functional fragment of said enzyme;
at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or 11 or a functional fragment of said enzyme;
at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

13. A method for synthesizing isoprene comprising:
enzymatically converting acetyl-CoA to acetoacetyl-CoA using a polypeptide having the activity of an acetyl-CoA acetyltransferase enzyme having the amino acid sequence set forth in SEQ ID No: 1 or 9 or a functional fragment of said enzyme;
enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or 10 or a functional fragment of said enzyme;
enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or 9 or a functional fragment of said enzyme;
enzymatically converting (R)-mevalonate to (R)-5-phosphomevalonate using a polypeptide having the activity of a mevalonate-kinase enzyme having the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having the amino acid sequence set forth in SEQ ID No: 8 or 15 or a functional fragment of said enzyme.

14. A method for synthesizing isoprene comprising:
enzymatically converting acetyl-CoA to acetoacetyl-CoA using a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or 9 or a functional fragment of said enzyme;
enzymatically converting acetoacetyl-CoA to 3-hydroxy-3-methylglutaryl-CoA using a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 2 or 10 or a functional fragment of said enzyme;
enzymatically converting 3-hydroxy-3-methylglutaryl-CoA to (R)-mevalonate using a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 3 or 9 or a functional fragment of said enzyme;
enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

15. A method for synthesizing isoprene comprising:
enzymatically converting acetyl-CoA to acetoacetyl-CoA using a polypeptide having the activity of an acetyl-CoA acetyltransferase enzyme having the amino acid sequence set forth in SEQ ID No: 1 or 9 or a functional fragment of said enzyme;
enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or 10 or a functional fragment of said enzyme;
enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or 9 or a functional fragment of said enzyme;
enzymatically converting (R)-mevalonate to (R)-5-phosphomevalonate using a polypeptide having the activity of a mevalonate-kinase enzyme having the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme classified under EC 4.2.3.27 or a functional fragment of said enzyme.

16. The method of claim 15, wherein the isoprene synthase enzyme has at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

17. A method for synthesizing isoprene comprising:
enzymatically converting acetyl-CoA to acetoacetyl-CoA using a polypeptide having the activity of an acetyl-CoA acetyltransferase enzyme having the amino acid sequence set forth in SEQ ID No: 1 or 9 or a functional fragment of said enzyme;
enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 2 or 10 or a functional fragment of said enzyme;
enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 3 or 9 or a functional fragment of said enzyme;
enzymatically converting (R)-mevalonate to (R)-5-phosphomevalonate using a polypeptide having the activity of a mevalonate-kinase enzyme having the amino acid sequence set forth in SEQ ID No: 4 or 11 or a functional fragment of said enzyme;
enzymatically converting (R)-5-phosphomevalonate to (R)-5-diphosphomevalonate using a polypeptide having the activity of a phosphomevalonate kinase enzyme having the amino acid sequence set forth in SEQ ID No: 5 or 12 or a functional fragment of said enzyme;
enzymatically converting (R)-5-diphosphomevalonate to isopentenyl diphosphate using a polypeptide having the activity of a diphosphomevalonate decarboxylase enzyme having the amino acid sequence set forth in SEQ ID No: 6 or 13 or a functional fragment of said enzyme;
enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
enzymatically converting dimethylallyl diphosphate to isoprene using a polypeptide having the activity of an isoprene synthase enzyme having the amino acid sequence set forth in SEQ ID No: 8 or 15 or a functional fragment of said enzyme.

18. A method for synthesizing isoprene comprising:
converting isopentenyl diphosphate to dimethylallyl diphosphate using a polypeptide having the activity of an isopentenyl diphosphate isomerase enzyme having an amino acid sequence set forth in SEQ ID No: 7 or 14 or a functional fragment of said enzyme.

19. A non-naturally occurring host capable of producing isoprene via the mevalonate pathway, said host comprising:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a hydroxymethylglutaryl-CoA synthase enzyme having the amino acid sequence set forth in SEQ ID No: 2 or 10 or a functional fragment of said enzyme;
at least one exogenous nucleic acid encoding a polypeptide having the activity of a hydroxymethylglutaryl Co-A reductase enzyme having the amino acid sequence set forth in SEQ ID No: 3 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having the amino acid sequence set forth in SEQ ID No: 7 or 14 or a functional fragment of said enzyme; and
at least one exogenous nucleic acid encoding a polypeptide having the activity of an isoprene synthase enzyme having the amino acid sequence set forth in SEQ ID No: 8 or 15 or a functional fragment of said enzyme.

20. A non-naturally occurring host capable of producing isoprene via the mevalonate pathway, said host comprising:
at least one exogenous nucleic acid encoding a polypeptide having the activity of an acetyl-CoA acetyltransferase enzyme having at least 90%, 91%, 92%, 93%, 94%, 95%, 96%, 97%, 98%, or 99% sequence identity to the amino acid sequence set forth in SEQ ID No: 1 or 9 or a functional fragment of said enzyme;
at least one exogenous nucleic acid encoding a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 2 or 10 or a functional fragment of said enzyme;
at least one exogenous nucleic acid encoding a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 3 or 9 or a functional fragment of said enzyme;
at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 4 or 11 or a functional fragment of said enzyme;
at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

21. The host of claim 19 or 20, wherein at least one of the exogenous nucleic acids is contained within a plasmid.

22. The host of claim 19 or 20, wherein at least one of the exogenous nucleic acids is integrated into a chromosome of the host.

23. A non-naturally occurring host capable of producing isoprene via the mevalonate pathway, said host comprising at least one of:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a hydroxymethylglutaryl-CoA synthase enzyme having the amino acid sequence set forth in SEQ ID No: 2 or 10 or a functional fragment of said enzyme;
at least one exogenous nucleic acid encoding a polypeptide having the activity of a hydroxymethylglutaryl Co-A reductase enzyme having the amino acid sequence set forth in SEQ ID No: 3 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having the amino acid sequence set forth in SEQ ID No: 7 or 14 or a functional fragment of said enzyme; and
at least one exogenous nucleic acid encoding a polypeptide having the activity of an isoprene synthase enzyme having the amino acid sequence set forth in SEQ ID No: 8 or 15 or a functional fragment of said enzyme;
and said host further comprising at least one of:
at least one endogenous nucleic acid encoding a polypeptide having the activity of an acetyl-CoA acetyltransferase enzyme;
at least one endogenous nucleic acid encoding a polypeptide having the activity of a hydroxymethylglutaryl-CoA synthase enzyme;
at least one endogenous nucleic acid encoding a polypeptide having the activity of a hydroxymethylglutaryl Co-A reductase enzyme;
at least one endogenous nucleic acid encoding a polypeptide having the activity of a mevalonate-kinase enzyme;
at least one endogenous nucleic acid encoding a polypeptide having the activity of a phosphomevalonate kinase enzyme;
at least one endogenous nucleic acid encoding a polypeptide having the activity of a diphosphomevalonate decarboxylase enzyme;
at least one endogenous nucleic acid encoding a polypeptide having the activity of an isopentenyl diphosphate isomerase enzyme; and
at least one endogenous nucleic acid encoding a polypeptide having the activity of an isoprene synthase enzyme.

24. A non-naturally occurring host capable of producing isoprene via the mevalonate pathway, said host comprising:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a hydroxymethylglutaryl-CoA synthase enzyme having the amino acid sequence set forth in SEQ ID No: 2 or 10 or a functional fragment of said enzyme;
at least one exogenous nucleic acid encoding a polypeptide having the activity of a hydroxymethylglutaryl Co-A reductase enzyme having the amino acid sequence set forth in SEQ ID No: 3 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having the amino acid sequence set forth in SEQ ID No: 7 or 14 or a functional fragment of said enzyme; and
at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

25. A non-naturally occurring host capable of producing isoprene via the mevalonate pathway, said host comprising:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a hydroxymethylglutaryl-CoA synthase enzyme having the amino acid sequence set forth in SEQ ID No: 2 or 10 or a functional fragment of said enzyme;
at least one exogenous nucleic acid encoding a polypeptide having the activity of a hydroxymethylglutaryl Co-A reductase enzyme having the amino acid sequence set forth in SEQ ID No: 3 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having the amino acid sequence set forth in SEQ ID No: 7 or 14 or a functional fragment of said enzyme; and
at least one exogenous nucleic acid encoding a polypeptide having the activity of an isoprene synthase enzyme classified under EC 4.2.3.27 or a functional fragment of said enzyme.

26. The non-naturally occurring host of claim 25, wherein the at least one exogenous nucleic acid encoding a polypeptide having the activity of an isoprene synthase enzyme classified under EC 4.2.3.27 encodes an isoprene synthase enzyme having at least 90%, 91%, 92%, 93%, 94%, 95%, 96%, 97%, 98%, or 99% sequence identity to the amino acid sequence set forth in SEQ ID No: 8 or 15 or a functional fragment of said enzyme.

27. A non-naturally occurring host capable of producing isoprene via the mevalonate pathway, said host comprising:
at least one exogenous nucleic acid encoding a polypeptide having the activity of an acetyl-CoA acetyltransferase enzyme having the amino acid sequence set forth in SEQ ID No: 1 or 9 or a functional fragment of said enzyme;
at least one exogenous nucleic acid encoding a polypeptide having the activity of a hydroxymethylglutaryl-CoA synthase enzyme having the amino acid sequence set forth in SEQ ID No: 2 or 10 or a functional fragment of said enzyme;
at least one exogenous nucleic acid encoding a polypeptide having the activity of a hydroxymethylglutaryl Co-A reductase enzyme having the amino acid sequence set forth in SEQ ID No: 3 or 9 or a functional fragment of said enzyme;
at least one exogenous nucleic acid encoding a polypeptide having the activity of a mevalonate-kinase enzyme having the amino acid sequence set forth in SEQ ID No: 4 or 11 or a functional fragment of said enzyme;
at least one exogenous nucleic acid encoding a polypeptide having the activity of a phosphomevalonate kinase enzyme having the amino acid sequence set forth in SEQ ID No: 5 or 12 or a functional fragment of said enzyme;
at least one exogenous nucleic acid encoding a polypeptide having the activity of a diphosphomevalonate decarboxylase enzyme having the amino acid sequence set forth in SEQ ID No: 6 or 13 or a functional fragment of said enzyme;
at least one exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme; and
at least one exogenous nucleic acid encoding a polypeptide having the activity of an isoprene synthase enzyme having the amino acid sequence set forth in SEQ ID No: 8 or 15 or a functional fragment of said enzyme.

28. A non-naturally occurring host comprising a polypeptide having the activity of an exogenous nucleic acid encoding an isopentenyl diphosphate isomerase enzyme having the amino acid sequence set forth in SEQ ID No: 7 or 14 or a functional fragment of said enzyme.

29. A non-naturally occurring host comprising an exogenous nucleic acid encoding a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme.

30. The host of claim 28 or 29, wherein the exogenous nucleic acid is contained within a plasmid.

31. The host of claim 28 or 29, wherein the exogenous nucleic acid is integrated into a chromosome of the host.

32. A non-naturally occurring host capable of expressing a polypeptide having the activity of an isopentenyl diphosphate isomerase enzyme having the amino acid sequence set forth in SEQ ID No: 7 or 14 or a functional fragment of said enzyme.

33. A non-naturally occurring host capable of expressing a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No: 7 or 14 or a functional fragment of said enzyme.

34. The method of any of claim 1-6 or 13-18, wherein said method is performed in a recombinant host.

35. The method of any of claim 1-6 or 13-18, wherein at least one of the enzymatic conversions is performed in a recombinant host.

36. The host of any of claim 7-12 or 19-33, or the method of any of claims 34-35, wherein the host is a prokaryotic host.

37. The host of any of claim 7-12 or 19-33, or the method of any of claims 34-35, wherein the host is a prokaryotic host from the genus Escherichia, Clostridia, Corynebacteria, Cupriavidus, Pseudomonas, Bacillus, or Rhodococcus.

38. The host of any of claim 7-12 or 19-33, or the method of any of claims 34-35, wherein the host is from the genus Cupriavidus.

39. The host of any of claim 7-12 or 19-33, or the method of any of claims 34-35, wherein the host is Cupriavidus necator.

40. The host of any of claim 7-12 or 19-33, or the method of any of claims 34-35, wherein the host is a eukaryotic host.

41. The host of any of claim 7-12 or 19-33, or the method of any of claims 34-35, wherein the host is a eukaryotic host from the genus Aspergillus, Saccharomyces, Pichia, Yarrowia, Issatchenkia, Debaryomyces, Arxula, or Kluyveromyces.

42. The host of any of claim 7-12 or 19-33, or the method of any of claims 34-35, wherein the host is capable of endogenously producing isoprene via a non-mevalonate pathway.

43. The host of any of claim 7-12 or 19-33, or the method of any of claims 34-35, wherein at least one of the enzymatic conversions comprises gas fermentation within the host.

44. The host or method of claim 43, wherein the gas comprises at least one of natural gas, syngas, CO₂/H₂, methanol, ethanol, non-volatile residue, caustic wash from cyclohexane oxidation processes, or waste stream from a chemical or petrochemical industry.

45. The host or method of claim 44, wherein the gas is CO₂/H₂.

46. A method for synthesizing isoprene via the mevalonate pathway comprising culturing the host of any of claim 7-12 or 19-33 in a gas medium.

47. The method of claim 46, further comprising recovering the produced isoprene.

48. The method of claim 47, wherein the host performs the enzymatic synthesis by gas fermentation.

49. The method of claim 48, wherein the gas comprises at least one of natural gas, syngas, CO₂/H₂, methanol, ethanol, non-volatile residue, caustic wash from cyclohexane oxidation processes, or waste stream from a chemical or petrochemical industry.

50. The method of claim 49, wherein the gas is CO₂/H₂.

51. A method for synthesizing dimethylallyl diphosphate in a chemolithotrophic host comprising:
enzymatically converting (R)-mevalonate to (R)-5-phosphomevalonate using a polypeptide having the activity of a mevalonate-kinase enzyme having the amino acid sequence set forth in SEQ ID 11 or a functional fragment of said enzyme;
enzymatically converting (R)-5-phosphomevalonate to (R)-5-diphosphomevalonate using a polypeptide having the activity of a phosphomevalonate kinase enzyme having the amino acid sequence set forth in SEQ ID No: 12 or a functional fragment of said enzyme;
enzymatically converting (R)-5-diphosphomevalonate to isopentenyl diphosphate using a polypeptide having the activity of a diphosphomevalonate decarboxylase enzyme having the amino acid sequence set forth in SEQ ID No: 13 or a functional fragment of said enzyme; and
enzymatically converting isopentenyl diphosphate to dimethylallyl diphosphate using a polypeptide having the activity of an isopentenyl diphosphate isomerase enzyme having the amino acid sequence set forth in SEQ ID No 14 or a functional fragment of said enzyme.

52. A method for synthesizing dimethylallyl diphosphate in a chemolithotrophic host comprising:
enzymatically converting (R)-mevalonate to (R)-5-phosphomevalonate using a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID 11 or a functional fragment of said enzyme;
enzymatically converting (R)-5-phosphomevalonate to (R)-5-diphosphomevalonate using a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 12 or a functional fragment of said enzyme;
enzymatically converting (R)-5-diphosphomevalonate to isopentenyl diphosphate using a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 13 or a functional fragment of said enzyme; and
enzymatically converting isopentenyl diphosphate to dimethylallyl diphosphate using a polypeptide having the activity of an isopentenyl diphosphate isomerase enzyme having at least 90%, 91%, 92%, 93%, 94%, 95%, 96%, 97%, 98%, or 99% sequence identity to the amino acid sequence set forth in SEQ ID No 14 or a functional fragment of said enzyme.

53. A method for synthesizing isoprene, comprising enzymatically converting dimethylallyl diphosphate synthesized according to the method of any of claims 51-52 to isoprene using a polypeptide having the activity of an isoprene synthase enzyme having the amino acid sequence set forth in SEQ ID No: 15 or a functional fragment of said enzyme.

54. A method for synthesizing isoprene, comprising enzymatically converting dimethylallyl diphosphate synthesized according to the method of any of claims 51-52 to isoprene using a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 15 or a functional fragment of said enzyme.

55. A non-naturally occurring chemolithotrophic host capable of producing dimethylallyl diphosphate via the lower mevalonate pathway, said host comprising:
at least one exogenous nucleic acid encoding a polypeptide having the activity of a mevalonate-kinase enzyme having the amino acid sequence set forth in SEQ ID No: 11 or a functional fragment of said enzyme;
at least one exogenous nucleic acid encoding a polypeptide having the activity of a phosphomevalonate kinase enzyme having the amino acid sequence set forth in SEQ ID No: 12 or a functional fragment of said enzyme;
at least one exogenous nucleic acid encoding a polypeptide having the activity of a diphosphomevalonate decarboxylase enzyme having the amino acid sequence set forth in SEQ ID No: 13 or a functional fragment of said enzyme; and
at least one exogenous nucleic acid encoding a polypeptide having the activity of an isopentenyl diphosphate isomerase enzyme having the amino acid sequence set forth in SEQ ID No: 14 or a functional fragment of said enzyme.

56. A non-naturally occurring chemolithotrophic host capable of producing dimethylallyl diphosphate via the lower mevalonate pathway, said host comprising:
at least one exogenous nucleic acid encoding a polypeptide having the activity of a mevalonate-kinase enzyme having at least 90%, 91%, 92%, 93%, 94%, 95%, 96%, 97%, 98%, or 99% sequence identity to the amino acid sequence set forth in SEQ ID No: 11 or a functional fragment of said enzyme;
at least one exogenous nucleic acid encoding a polypeptide having the activity of a phosphomevalonate kinase enzyme having at least 90%, 91%, 92%, 93%, 94%, 95%, 96%, 97%, 98%, or 99% sequence identity to the amino acid sequence set forth in SEQ ID No: 12 or a functional fragment of said enzyme;
at least one exogenous nucleic acid encoding a polypeptide having the activity of a diphosphomevalonate decarboxylase enzyme having at least 90%, 91%, 92%, 93%, 94%, 95%, 96%, 97%, 98%, or 99% sequence identity to the amino acid sequence set forth in SEQ ID No: 13 or a functional fragment of said enzyme; and
at least one exogenous nucleic acid encoding a polypeptide having the activity of an isopentenyl diphosphate isomerase enzyme having at, least 90%, 91%, 92%, 93%, 94%, 95%, 96%, 97%, 98%, or 99% sequence identity to the amino acid sequence set forth in SEQ ID No: 14 or a functional fragment of said enzyme.

57. The host of any of claims 55-56, further comprising at least one exogenous nucleic acid encoding a polypeptide having the activity of an isoprene synthase enzyme having the amino acid sequence set forth in SEQ ID No: 15 or a functional fragment of said enzyme.

58. The host of any of claims 55-56, further comprising at least one exogenous nucleic acid encoding a polypeptide having the activity of an isoprene synthase enzyme having at least 90%, 91%, 92%, 93%, 94%, 95%, 96%, 97%, 98%, or 99% sequence identity to the amino acid sequence set forth in SEQ ID No: 15 or a functional fragment of said enzyme.

59. The method of any of claims 51-54, wherein said method is performed in a recombinant host.

60. The host of any of claims 55-58, or the method of claim 59, wherein at least one of the exogenous nucleic acids is contained within a plasmid.

61. The host of any of claims 55-58, or the method of claim 59, wherein the host is from the genus Cupriavidus.

62. The host of any of claims 55-58, or the method of claim 59, wherein the host is Cupriavidus necator.

63. A method for synthesizing mevalonate in a chemolithotrophic host comprising:
enzymatically converting acetyl-CoA to acetoacetyl-CoA using a polypeptide having the activity of an acetoacetyl-CoA C-acetyltransferase enzyme having the amino acid sequence set forth in SEQ ID No: 9 or a functional fragment of said enzyme;
enzymatically converting acetoacetyl-CoA to 3-hydroxy-3-methylglutaryl-CoA using a polypeptide having the activity of a hydroxymethylglutaryl-CoA synthase enzyme having the amino acid sequence set forth in SEQ ID No: 10 or a functional fragment of said enzyme; and
enzymatically converting 3-hydroxy-3-methylglutaryl-CoA to (R)-mevalonate using a polypeptide having the activity of a hydroxymethylglutaryl Co-A reductase enzyme having the amino acid sequence set forth in SEQ ID No: 9 or a functional fragment of said enzyme.

64. A method for synthesizing mevalonate in a chemolithotrophic host comprising:
enzymatically converting acetyl-CoA to acetoacetyl-CoA using a polypeptide having the activity of an acetoacetyl-CoA C-acetyltransferase enzyme having at least 90%, 91%, 92%, 93%, 94%, 95%, 96%, 97%, 98%, or 99% sequence identity to the amino acid sequence set forth in SEQ ID No: 9 or a functional fragment of said enzyme;
enzymatically converting acetoacetyl-CoA to 3-hydroxy-3-methylglutaryl-CoA using a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 10 or a functional fragment of said enzyme; and
enzymatically converting 3-hydroxy-3-methylglutaryl-CoA to (R)-mevalonate using a polypeptide having the activity of a hydroxymethylglutaryl Co-A reductase enzyme having at least 90%, 91%, 92%, 93%, 94%, 95%, 96%, 97%, 98%, or 99% sequence identity to the amino acid sequence set forth in SEQ ID No: 9 or a functional fragment of said enzyme.

65. A non-naturally occurring chemolithotrophic host capable of producing mevalonate via the upper mevalonate pathway, said host comprising:
at least one exogenous nucleic acid encoding a polypeptide having the activity of an enzyme having the amino acid sequence set forth in SEQ ID No: 9 or a functional fragment of said enzyme; and
at least one exogenous nucleic acid encoding a polypeptide having the activity of a hydroxymethylglutaryl-CoA synthase enzyme having the amino acid sequence set forth in SEQ ID No: 10 or a functional fragment of said enzyme.

66. A non-naturally occurring chemolithotrophic host capable of producing mevalonate via the upper mevalonate pathway, said host comprising:
at least one exogenous nucleic acid encoding a polypeptide having the activity of an enzyme having at least 90%, 91%, 92%, 93%, 94%, 95%, 96%, 97%, 98%, or 99% sequence identity to the amino acid sequence set forth in SEQ ID No: 9 or a functional fragment of said enzyme; and
at least one exogenous nucleic acid encoding a polypeptide having the activity of a hydroxymethylglutaryl-CoA synthase enzyme having at least 90%, 91%, 92%, 93%, 94%, 95%, 96%, 97%, 98%, or 99% sequence identity to the amino acid sequence set forth in SEQ ID No: 10 or a functional fragment of said enzyme.

67. The method of any of claims 63-64, wherein said method is performed in a recombinant host.

68. The host of any of claims 65-66, or the method of claim 67, wherein at least one of the exogenous nucleic acids is contained within a plasmid.

69. The host of any of claims 65-66, or the method of claim 67, wherein the host is from the genus Cupriavidus.

70. The host of any of claims 65-66, or the method of claim 67, wherein the host is Cupriavidus necator.

71. A non-naturally occurring mutant or variant of SEQ ID No: 7 or 14 comprising one or more non-naturally occurring mutations, wherein the mutant or variant exhibits isopentenyl diphosphate isomerase activity.

72. An isolated polypeptide comprising an amino acid sequence having 90%, 91%, 92%, 93%, 94%, 95%, 96%, 97%, 98%, or 99% sequence identity to the amino acid sequence set forth in SEQ ID No: 7 or 14 or a functional fragment of said polypeptide, wherein the polypeptide exhibits isopentenyl diphosphate isomerase activity.

73. A nucleic acid encoding an isopentenyl diphosphate isomerase enzyme having 90%, 91%, 92%, 93%, 94%, 95%, 96%, 97%, 98%, or 99% sequence identity to the amino acid sequence set forth in SEQ ID No: 7 or 14 or a functional fragment of said enzyme.

74. A composition for producing isoprene comprising the host of any of claim 7-12 or 19-33.

75. A composition comprising isoprene synthesized by the method of any of claim 1-6, 13-18, 34, or 35.

76. A composition comprising an isopentenyl diphosphate isomerase enzyme having the amino acid sequence set forth in SEQ ID No: 7 or 14 or having at least 90%, 91%, 92%, 93%, 94%, 95%, 96%, 97%, 98%, or 99% sequence identity to the amino acid sequence set forth in SEQ ID No: 7 or 14 or a functional fragment of said enzyme, and further means for enzymatically producing isoprene from a suitable substrate.

77. A composition comprising a substrate, a polypeptide having the activity of an isopentenyl diphosphate isomerase enzyme having the amino acid sequence set forth in SEQ ID No: 7 or 14 or having at least 90%, 91%, 92%, 93%, 94%, 95%, 96%, 97%, 98%, or 99% sequence identity to the amino acid sequence set forth in SEQ ID No: 7 or 14 or a functional fragment of said enzyme, and further means for enzymatically producing isoprene from said substrate.

78. A method for producing bioderived isoprene, comprising culturing or growing a host according to of any of claim 7-12 or 19-33 under conditions and for a sufficient period of time to produce bioderived isoprene.

79. Bioderived isoprene produced in a host according to any of claim 7-12 or 19-33, wherein said bioderived isoprene has a carbon-12, carbon-13, and carbon-14 isotope ratio that reflects an atmospheric carbon dioxide uptake source.

80. A bio-derived, bio-based, or fermentation-derived product produced from any of the methods or hosts of claims 1 to 70, wherein said product comprises:
i. a composition comprising at least one bio-derived, bio-based, or fermentation-derived compound or any combination thereof,
ii. a bio-derived, bio-based, or fermentation-derived polymer comprising the bio-derived, bio-based, or fermentation-derived composition or compound of i., or any combination thereof,
iii. a bio-derived, bio-based, or fermentation-derived cis-polyisoprene rubber, trans-polyisoprene rubber, or liquid polyisoprene rubber, comprising the bio-derived, bio-based, or fermentation-derived compound or bio-derived, bio-based, or fermentation-derived composition of i., or any combination thereof or the bio-derived, bio-based, or fermentation-derived polymer of ii., or any combination thereof,
iv. a molded substance obtained by molding the bio-derived, bio-based, or fermentation-derived polymer of or the bio-derived, bio-based, or fermentation-derived resin of iii., or any combination thereof,
v. a bio-derived, bio-based, or fermentation-derived formulation comprising the bio-derived, bio-based, or fermentation-derived composition of i., bio-derived, bio-based, or fermentation-derived compound of i., bio-derived, bio-based, or fermentation-derived polymer of ii., bio-derived, bio-based, or fermentation-derived resin of iii., or bio-derived, bio-based, or fermentation-derived molded substance of iv, or any combination thereof, or
vi. a bio-derived, bio-based, or fermentation-derived semi-solid or a non-semi-solid stream, comprising the bio-derived, bio-based, or fermentation-derived composition of i., bio-derived, bio-based, or fermentation-derived compound of i., bio-derived, bio-based, or fermentation-derived polymer of ii., bio-derived, bio-based, or fermentation-derived resin of iii., bio-derived, bio-based, or fermentation-derived formulation of v., or bio-derived, bio-based, or fermentation-derived molded substance of iv., or any combination thereof.
